SEQUENCE LISTING (68 sequences)

SEQ ID NO: 1 - DNA, 73 bp, synthetic construct
  misc_feature 1..73: note = Description of sequence: artificial oligionucleotide
  source 1..73: mol_type = other DNA
  variation 19..48: note = n is a, c, g, or t
ggaggctctcgggacgacnnnnnnnnnnnnnnnnnnnnnnnnnnnnnngtcgtcccgcctttaggatttacag

SEQ ID NO: 2 - DNA, 83 bp, synthetic construct
  misc_feature 1..83: note = Description of sequence: artificial oligionucleotide
  source 1..83: mol_type = other DNA
  variation 19..58: note = n is a, c, g, or t
ggaggctctcgggacgacnnnnnnnnnnnnnnnnnnnnnnnnnnnnnnnnnnnnnnnngtcgtcccgcctttaggatttacag

SEQ ID NO: 3 - DNA, 93 bp, synthetic construct
  misc_feature 1..93: note = Description of sequence: artificial oligionucleotide
  source 1..93: mol_type = other DNA
  variation 24..63: note = n is a, c, g, or t
tccatagctccttgttcaaatctnnnnnnnnnnnnnnnnnnnnnnnnnnnnnnnnnnnnnnnnagtctagatttgaattctcggatacaacga

SEQ ID NO: 4 - DNA, 18 bp, synthetic construct
  misc_feature 1..18: note = Description of sequence: artificial oligionucleotide
  source 1..18: mol_type = other DNA
ggaggctctcgggacgac

SEQ ID NO: 5 - DNA, 25 bp, synthetic construct
  misc_feature 1..25: note = Description of sequence: artificial oligionucleotide
  source 1..25: mol_type = other DNA
  modified_base 1: mod_base = OTHER; note = base is biotinylated
ctgtaaatcctaaaggcgggacgac

SEQ ID NO: 6 - DNA, 18 bp, synthetic construct
  misc_feature 1..18: note = Description of sequence: artificial oligionucleotide
  source 1..18: mol_type = other DNA
  modified_base 3: mod_base = OTHER; note = base is biotinylated
gtcgtcccgagagccata

SEQ ID NO: 7 - DNA, 96 bp, synthetic construct
  misc_feature 1..96: note = Description of sequence: artificial oligionucleotide
  source 1..96: mol_type = other DNA
  variation 34..78: note = n is a, c, g, or t
acactctttccctacacgacgctcttccgatctnnnnnnnnnnnnnnnnnnnnnnnnnnnnnnnnnnnnnnnnnnnnnggaggctctcgggacgac

SEQ ID NO: 8 - DNA, 100 bp, synthetic construct
  misc_feature 1..100: note = Description of sequence: artificial oligionucleotide
  source 1..100: mol_type = other DNA
  variation 33..75: note = n is a, c, g, or t
gactggagttcagacgtgtgctcttccgatctnnnnnnnnnnnnnnnnnnnnnnnnnnnnnnnnnnnnnnnnnnnctgtaaatcctaaaggcgggacgac

SEQ ID NO: 9 - DNA, 23 bp, synthetic construct
  misc_feature 1..23: note = Description of sequence: artificial oligionucleotide
  source 1..23: mol_type = other DNA
tccatagctccttgttcaaatct

SEQ ID NO: 10 - DNA, 25 bp, synthetic construct
  misc_feature 1..25: note = Description of sequence: artificial oligionucleotide
  source 1..25: mol_type = other DNA
  modified_base 1: mod_base = OTHER; note = base is biotinylated
tcgttgtatccgagaattcaaatct

SEQ ID NO: 11 - DNA, 15 bp, synthetic construct
  misc_feature 1..15: note = Description of sequence: artificial oligionucleotide
  source 1..15: mol_type = other DNA
  modified_base 1: mod_base = OTHER; note = base is biotinylated
cgagaattcaaatct

SEQ ID NO: 12 - DNA, 97 bp, synthetic construct
  misc_feature 1..97: note = Description of sequence: artificial oligionucleotide
  source 1..97: mol_type = other DNA
  variation 34..77: note = n is a, c, g, or t
acactctttccctacacgacgctcttccgatctnnnnnnnnnnnnnnnnnnnnnnnnnnnnnnnnnnnnnnnnnnnntccatagctccttgttcaaa

SEQ ID NO: 13 - DNA, 97 bp, synthetic construct
  misc_feature 1..97: note = Description of sequence: artificial oligionucleotide
  source 1..97: mol_type = other DNA
  variation 33..77: note = n is a, c, g, or t
gactggagttcagacgtgtgctcttccgatctnnnnnnnnnnnnnnnnnnnnnnnnnnnnnnnnnnnnnnnnnnnnntcgttgtatccgagaattca

SEQ ID NO: 14 - DNA, 84 bp, synthetic construct
  misc_feature 1..84: note = Description of sequence: artificial oligionucleotide
  source 1..84: mol_type = other DNA
actggagctcaatcagtacacgacggttgcaagtgggactctggtaggctgggttaatttgggacaagcttcaaccatggagta

SEQ ID NO: 15 - DNA, 40 bp, synthetic construct
  misc_feature 1..40: note = Description of sequence: artificial oligionucleotide
  source 1..40: mol_type = other DNA
acggttgcaagtgggactctggtaggctgggttaatttgg

SEQ ID NO: 16 - DNA, 82 bp, synthetic construct
  misc_feature 1..82: note = Description of sequence: artificial oligionucleotide
  source 1..82: mol_type = other DNA
aggaattcagatctccctgcaggtggtgttttttttgtgtgctgtgtttttgtgatgcatgctcgaggagctcaggatcccg

SEQ ID NO: 17 - DNA, 75 bp, synthetic construct
  misc_feature 1..75: note = Description of sequence: artificial oligionucleotide
  source 1..75: mol_type = other DNA
atacgagcttgttcaatagcgtttctatctggctgtatcgtgatagtaagagcactaatgatagtaagagcaatc

SEQ ID NO: 18 - DNA, 46 bp, synthetic construct
  misc_feature 1..46: note = Description of sequence: artificial oligionucleotide
  source 1..46: mol_type = other DNA
gggacgaccccattagtggtgccgggtgtttatggtgcgtcgtccc

SEQ ID NO: 19 - DNA, 46 bp, synthetic construct
  misc_feature 1..46: note = Description of sequence: artificial oligionucleotide
  source 1..46: mol_type = other DNA
gggacgaccactttgggcaggtaggttgggcaagtagtgtcgtccc

SEQ ID NO: 20 - DNA, 46 bp, synthetic construct
  misc_feature 1..46: note = Description of sequence: artificial oligionucleotide
  source 1..46: mol_type = other DNA
gggacgacccagttgggtaggtatggttgggcacggtggtcgtccc

SEQ ID NO: 21 - DNA, 46 bp, synthetic construct
  misc_feature 1..46: note = Description of sequence: artificial oligionucleotide
  source 1..46: mol_type = other DNA
gggacgacggcacggttagcatggctgtatttgtgtaggtcgtccc

SEQ ID NO: 22 - DNA, 46 bp, synthetic construct
  misc_feature 1..46: note = Description of sequence: artificial oligionucleotide
  source 1..46: mol_type = other DNA
gggacgaccacggttaaagtagcactttgtatttgggtgtcgtccc

SEQ ID NO: 23 - DNA, 46 bp, synthetic construct
  misc_feature 1..46: note = Description of sequence: artificial oligionucleotide
  source 1..46: mol_type = other DNA
gggacgacgctaggggatcttttggggtttgtatgggcgtcgtccc

SEQ ID NO: 24 - DNA, 46 bp, synthetic construct
  misc_feature 1..46: note = Description of sequence: artificial oligionucleotide
  source 1..46: mol_type = other DNA
gggacgacgaggggatccatccaatggggtttgtatacgtcgtccc

SEQ ID NO: 25 - DNA, 46 bp, synthetic construct
  misc_feature 1..46: note = Description of sequence: artificial oligionucleotide
  source 1..46: mol_type = other DNA
gggacgacggtacccgccggtgcaggatctggggtggtgtcgtccc

SEQ ID NO: 26 - DNA, 46 bp, synthetic construct
  misc_feature 1..46: note = Description of sequence: artificial oligionucleotide
  source 1..46: mol_type = other DNA
gggacgacggcaattgccggtgcaggatctgaattatcgtcgtccc

SEQ ID NO: 27 - DNA, 56 bp, synthetic construct
  misc_feature 1..56: note = Description of sequence: artificial oligionucleotide
  source 1..56: mol_type = other DNA
gggacgacgggatggtaagtggtaggtcgttggaatttctactgtatcgtcgtccc

SEQ ID NO: 28 - DNA, 56 bp, synthetic construct
  misc_feature 1..56: note = Description of sequence: artificial oligionucleotide
  source 1..56: mol_type = other DNA
gggacgacgggacggtaagtggtaggtcgttggaatttctactgtatcgtcgtccc

SEQ ID NO: 29 - DNA, 56 bp, synthetic construct
  misc_feature 1..56: note = Description of sequence: artificial oligionucleotide
  source 1..56: mol_type = other DNA
gggacgaccgtccgagttttaggagtggttgcattgcgctgggaagaggtcgtccc

SEQ ID NO: 30 - DNA, 56 bp, synthetic construct
  misc_feature 1..56: note = Description of sequence: artificial oligionucleotide
  source 1..56: mol_type = other DNA
gggacgacgcactatggctagggttggtacggtcgtgaaccgcggggtgtcgtccc

SEQ ID NO: 31 - DNA, 56 bp, synthetic construct
  misc_feature 1..56: note = Description of sequence: artificial oligionucleotide
  source 1..56: mol_type = other DNA
gggacgacggcacggggttattcgttggttgcactgcgctgggaggcagtcgtccc

SEQ ID NO: 32 - DNA, 56 bp, synthetic construct
  misc_feature 1..56: note = Description of sequence: artificial oligionucleotide
  source 1..56: mol_type = other DNA
gggacgacgggatggtaagtggcaggtcgttggaatttctactgtatcgtcgtccc

SEQ ID NO: 33 - DNA, 56 bp, synthetic construct
  misc_feature 1..56: note = Description of sequence: artificial oligionucleotide
  source 1..56: mol_type = other DNA
gggacgacggcacgaggttattcgttggttgcactgcgctgggaggtagtcgtccc

SEQ ID NO: 34 - DNA, 56 bp, synthetic construct
  misc_feature 1..56: note = Description of sequence: artificial oligionucleotide
  source 1..56: mol_type = other DNA
gggacgacggggctcttaccctggagggtagaaggggaggtgtggtcagtcgtccc

SEQ ID NO: 35 - DNA, 56 bp, synthetic construct
  misc_feature 1..56: note = Description of sequence: artificial oligionucleotide
  source 1..56: mol_type = other DNA
gggacgacggcacgtggttattcgttggttgcactgcgctgggaggcagtcgtccc

SEQ ID NO: 36 - DNA, 56 bp, synthetic construct
  misc_feature 1..56: note = Description of sequence: artificial oligionucleotide
  source 1..56: mol_type = other DNA
gggacgaccggctgtctggatgcattgcgccgggaactcggacggatggtcgtccc

SEQ ID NO: 37 - DNA, 56 bp, synthetic construct
  misc_feature 1..56: note = Description of sequence: artificial oligionucleotide
  source 1..56: mol_type = other DNA
gggacgaccgtccgagttataggagtggttgcattgcgctgggaagaggtcgtccc

SEQ ID NO: 38 - DNA, 56 bp, synthetic construct
  misc_feature 1..56: note = Description of sequence: artificial oligionucleotide
  source 1..56: mol_type = other DNA
gggacgacggcacgaggttattcgttggttgcactgcgctgggaggaagtcgtccc

SEQ ID NO: 39 - DNA, 56 bp, synthetic construct
  misc_feature 1..56: note = Description of sequence: artificial oligionucleotide
  source 1..56: mol_type = other DNA
gggacgacgggacggtaagtggcaggtcgttggaatttctactgtatcgtcgtccc

SEQ ID NO: 40 - DNA, 56 bp, synthetic construct
  misc_feature 1..56: note = Description of sequence: artificial oligionucleotide
  source 1..56: mol_type = other DNA
gggacgacggcacgaggttattcgttggttgcactgcgctgggaggcagtcgtccc

SEQ ID NO: 41 - DNA, 56 bp, synthetic construct
  misc_feature 1..56: note = Description of sequence: artificial oligionucleotide
  source 1..56: mol_type = other DNA
gggacgacggcacgaggttaatcgttggttgcactgcgctgggaggcagtcgtccc

SEQ ID NO: 42 - DNA, 56 bp, synthetic construct
  misc_feature 1..56: note = Description of sequence: artificial oligionucleotide
  source 1..56: mol_type = other DNA
gggacgacggcacgaggtaattcgttggttgcactgcgctgggaggcagtcgtccc

SEQ ID NO: 43 - DNA, 63 bp, synthetic construct
  misc_feature 1..63: note = Description of sequence: artificial oligionucleotide
  source 1..63: mol_type = other DNA
ttcaaatctgagcagcctagttactgacgggtttatagtactcgcattgagtctagatttgaa

SEQ ID NO: 44 - DNA, 63 bp, synthetic construct
  misc_feature 1..63: note = Description of sequence: artificial oligionucleotide
  source 1..63: mol_type = other DNA
ttcaaatctggtgccctttcgcagtcggttggtaatgagaccactttagagtctagatttgaa

SEQ ID NO: 45 - DNA, 63 bp, synthetic construct
  misc_feature 1..63: note = Description of sequence: artificial oligionucleotide
  source 1..63: mol_type = other DNA
ttcaaatcttatgtacgttctatcgcatggatgggattttgggttacctagtctagatttgaa

SEQ ID NO: 46 - DNA, 63 bp, synthetic construct
  misc_feature 1..63: note = Description of sequence: artificial oligionucleotide
  source 1..63: mol_type = other DNA
ttcaaatcttggtacgttctgccttttcttattttgctttgggttacctagtctagatttgaa

SEQ ID NO: 47 - DNA, 63 bp, synthetic construct
  misc_feature 1..63: note = Description of sequence: artificial oligionucleotide
  source 1..63: mol_type = other DNA
ttcaaatctatggtgcagtgcgcagggacggaggtaaaacatgagttttagtctagatttgaa

SEQ ID NO: 48 - DNA, 63 bp, synthetic construct
  misc_feature 1..63: note = Description of sequence: artificial oligionucleotide
  source 1..63: mol_type = other DNA
ttcaaatctgagcagcctagttactgacgggtttatagtactcgcatcgagtctagatttgaa

SEQ ID NO: 49 - DNA, 63 bp, synthetic construct
  misc_feature 1..63: note = Description of sequence: artificial oligionucleotide
  source 1..63: mol_type = other DNA
ttcaaatctggtgccctttcgcagtcggttggtaatgagatcactttagagtctagatttgaa

SEQ ID NO: 50 - DNA, 63 bp, synthetic construct
  misc_feature 1..63: note = Description of sequence: artificial oligionucleotide
  source 1..63: mol_type = other DNA
ttcaaatctccacgctggactggatgcaatgcgccgggatatcgtctaaagtctagatttgaa

SEQ ID NO: 51 - DNA, 63 bp, synthetic construct
  misc_feature 1..63: note = Description of sequence: artificial oligionucleotide
  source 1..63: mol_type = other DNA
ttcaaatctgagcagcctagttactgacgggtttatagtactcacattgagtctagatttgaa

SEQ ID NO: 52 - DNA, 63 bp, synthetic construct
  misc_feature 1..63: note = Description of sequence: artificial oligionucleotide
  source 1..63: mol_type = other DNA
ttcaaatctgagcagcctagttactgacgggtttatagtactcgtattgagtctagatttgaa

SEQ ID NO: 53 - DNA, 65 bp, synthetic construct
  misc_feature 1..65: note = Description of sequence: artificial oligionucleotide
  source 1..65: mol_type = other DNA
ttcaaatctgcggcgactaattcttagtggtggttgcaatgcgctgggatgagtctagatttgaa

SEQ ID NO: 54 - DNA, 63 bp, synthetic construct
  misc_feature 1..63: note = Description of sequence: artificial oligionucleotide
  source 1..63: mol_type = other DNA
ttcaaatctgagcagcctagttactgacgggtttatagtactcgcactgagtctagatttgaa

SEQ ID NO: 55 - DNA, 63 bp, synthetic construct
  misc_feature 1..63: note = Description of sequence: artificial oligionucleotide
  source 1..63: mol_type = other DNA
ttcaaatcttatgtaagttctatcgcatggatgggattttgggttacctagtctagatttgaa

SEQ ID NO: 56 - DNA, 63 bp, synthetic construct
  misc_feature 1..63: note = Description of sequence: artificial oligionucleotide
  source 1..63: mol_type = other DNA
ttcaaatcttatgtacgtactatcgcatggatgggattttgggttacctagtctagatttgaa

SEQ ID NO: 57 - DNA, 63 bp, synthetic construct
  misc_feature 1..63: note = Description of sequence: artificial oligionucleotide
  source 1..63: mol_type = other DNA
ttcaaatcttatgtacgttctatcgcatggatgtgattttgggttacctagtctagatttgaa

SEQ ID NO: 58 - DNA, 63 bp, synthetic construct
  misc_feature 1..63: note = Description of sequence: artificial oligionucleotide
  source 1..63: mol_type = other DNA
ttcaaatcttatgtacgttctatcgcatggatgggattatgggttacctagtctagatttgaa

SEQ ID NO: 59 - DNA, 63 bp, synthetic construct
  misc_feature 1..63: note = Description of sequence: artificial oligionucleotide
  source 1..63: mol_type = other DNA
ttcaaatcttatgtacgttctatcgcatggatgggatttttggttacctagtctagatttgaa

SEQ ID NO: 60 - DNA, 63 bp, synthetic construct
  misc_feature 1..63: note = Description of sequence: artificial oligionucleotide
  source 1..63: mol_type = other DNA
ttcaaatcttatgtacgttctatcgcatggatgggattttgggttaccaagtctagatttgaa

SEQ ID NO: 61 - DNA, 63 bp, synthetic construct
  misc_feature 1..63: note = Description of sequence: oligonucleotide, name: ML4-mut1
  source 1..63: mol_type = other DNA
ttcaaatcttggtacgtactgccttttcttattttgctttgggttacctagtctagatttgaa

SEQ ID NO: 62 - DNA, 63 bp, synthetic construct
  misc_feature 1..63: note = Description of sequence: oligonucleotide, name: ML4-mut2
  source 1..63: mol_type = other DNA
ttcaaatcttggtacgttctgcattttcttattttgctttgggttacctagtctagatttgaa

SEQ ID NO: 63 - DNA, 63 bp, synthetic construct
  misc_feature 1..63: note = Description of sequence: oligonucleotide, name: ML4-mut3
  source 1..63: mol_type = other DNA
ttcaaatcttggtacgttctgcctattcttattttgctttgggttacctagtctagatttgaa

SEQ ID NO: 64 - DNA, 63 bp, synthetic construct
  misc_feature 1..63: note = Description of sequence: oligonucleotide, name: ML4-mut4
  source 1..63: mol_type = other DNA
ttcaaatcttggtacgttctgcctttacttattttgctttgggttacctagtctagatttgaa

SEQ ID NO: 65 - DNA, 63 bp, synthetic construct
  misc_feature 1..63: note = Description of sequence: oligonucleotide, name: ML4-mut5
  source 1..63: mol_type = other DNA
ttcaaatcttggtacgttctgccttttcttatattgctttgggttacctagtctagatttgaa

SEQ ID NO: 66 - DNA, 63 bp, synthetic construct
  misc_feature 1..63: note = Description of sequence: oligonucleotide, name: ML4-mut6
  source 1..63: mol_type = other DNA
ttcaaatcttggtacgttctgccttttcttatttagctttgggttacctagtctagatttgaa

SEQ ID NO: 67 - DNA, 63 bp, synthetic construct
  misc_feature 1..63: note = Description of sequence: oligonucleotide, name: ML4-mut7
  source 1..63: mol_type = other DNA
ttcaaatcttggtacgttctgccttttcttattttgctatgggttacctagtctagatttgaa

SEQ ID NO: 68 - DNA, 46 bp, synthetic construct
  misc_feature 1..46: note = Description of sequence: Aptamer for EAB sensor, name: LI3-45-MB
  source 1..46: mol_type = other DNA
  modified_base 1: mod_base = OTHER; note = thymine-S-S-C-C-C-C-C-C-thiol
  modified_base 46: mod_base = gm
tatgtacgttctatcgcatggatgggattttgggttacctagtctc

(19) United States
(12) Patent Application Publication
(10) Pub. No.: US 2026/0049321 A1
(43) Pub. Date: Feb. 19, 2026

(54) METHODS AND COMPOSITIONS OF APTAMERS FOR DETECTION OF LOW COMPLEXITY MOLECULES

(71) Applicant: North Carolina State University, Raleigh, NC (US)

(72) Inventors: Yi Xiao, Raleigh, NC (US); Linlin Wang, Gao'an City (CN)

(21) Appl. No.: 19/304,030

(22) Filed: Aug. 19, 2025

Related U.S. Application Data
(60) Provisional application No. 63/684,551, filed on Aug. 19, 2024.

(51) Int. Cl. C12N 15/115; C12Q 1/68

(52) U.S. Cl. CPC C12N 15/115; C12Q 1/68; C12N 2310/16

(57) ABSTRACT

Disclosed herein are methods and compositions for detecting (+)-methamphetamine using dye-complexed and redox-tag-labeled aptamers that generate a detectable signal upon target binding.

### CROSS REFERENCE TO RELATED APPLICATIONS

This application claims benefit of U.S. Provisional Application No. 63/684,551, filed Aug. 19, 2024, incorporated herein by reference in its entirety.

### GOVERNMENT SUPPORT CLAUSE

This invention was made with government support under grant number DA051100 awarded by the National Institutes of Health and 15PNIJ-22-GG-04440-RESS awarded by the National Institute of Justice. The government has certain rights in the invention.

### REFERENCE TO SEQUENCE LISTING

The sequence listing submitted on Aug. 19, 2025, as an .XML entitled “10620-160US1_ST26.xml” created on Aug. 19, 2025, and having a file size of 95,320 bytes is hereby incorporated by reference pursuant to 37 C.F.R. § 1.52(e)(5).

### BACKGROUND

Aptamers are oligonucleotide affinity reagents that can be synthesized at low cost, engineered by sequence design, and operated with robust thermal stability. These properties make them attractive alternatives to protein antibodies for sensing in medical diagnostics, health monitoring, food safety, and forensics. Relative to mass spectrometry, aptamer assays can reduce turnaround time and cost, which is important for point-of-care use.

Despite these advantages, isolating aptamers for small molecules remains difficult. Conventional SELEX workflows often immobilize the target on a solid support to enable partitioning, yet conjugation can mask the very few binding epitopes on low functionality targets and yield aptamers with poor affinity and specificity for the free, solution phase analyte. The literature illustrates these shortcomings for low complexity/low-epitope amine containing analytes. Multiple reported small molecule aptamers that were claimed to bind tightly by bead-based or indirect assays showed weak binding or no detectable binding when reexamined. In many cases, candidates also lacked selectivity and responded comparably to closely related aromatic amines and other interferents that commonly co-occur in real samples. These discrepancies highlight the risk of assay dependent overestimation of performance in existing workflows.

In addition to that, for many small molecule targets, available aptamers exhibit micromolar affinities, poor enantiomeric or structural discrimination, and cross reactivity to common interferents present in biological matrices. Furthermore, field-ready testing still relies heavily on immunoassays or laboratory mass spectrometry, which can suffer from cross reactivity, higher cost, instrument dependence, or longer turnaround, creating barriers to rapid screening of seized materials and near patient testing in fluids.

There is a need for affinity reagents and selection workflows that reproducibly deliver high affinity and high specificity recognition of low epitope small molecules in complex samples, with robust discrimination against closely related analogs and interferents. There is also a need for compositions and methods that integrate smoothly into simple, rapid, and inexpensive assay formats suitable for point of care or field deployment.

Thus, there remains a significant unmet need for molecular recognition systems capable of detecting low epitope small molecules in complex, real-world samples with both high specificity and high affinity. Current approaches often fail to deliver consistent performance across different sample types, show reduced selectivity in the presence of structurally similar compounds, or require multi-step, instrument-intensive workflows that limit their practicality outside specialized laboratories.

### SUMMARY

Disclosed herein are aptamers, methods, assays, and compositions for detecting low complexity analytes, including phenethylamine-class compounds such as (+)-methamphetamine. The aptamers are complexed with an aggregation-sensitive dye and produce a detectable optical signal or electrochemical signal upon analyte binding. Also disclosed are methods for detecting analytes in various sample types, aptamer-based colorimetric assays, and methods for isolating aptamers with high affinity and specificity for the target compound.

In one aspect, disclosed herein is an aptamer comprising a nucleic acid that specifically binds to a phenethylamine-class compound, wherein the aptamer exhibits less than 25% cross-reactivity to a structural analog or an enantiomer of the phenethylamine-class compound, and a detectable signal that is detectable upon binding of the nucleic acid to the phenethylamine-class compound. Also disclosed herein, the phenethylamine-class compound is (+)-methamphetamine. Also disclosed herein, the aptamer comprises a nucleic acid sequence as set forth in any one of SEQ ID NOS: 45, 46, 57, 64, 65, and 66, and the nucleic acid sequence may comprise at least one additional mutation and/or at least one additional non-natural nucleic acid. In some embodiments, the at least one additional mutation of a thymine at a position corresponding to T18 or T39 within the nucleic acid sequence decreases affinity for the phenethylamine-class compound binding by at least 20-fold. Also disclosed herein, the nucleic acid may comprise a randomized region that includes at least 40 nucleotides, wherein the nucleotides are thymine rich with a thymine content of at least 40 percent. In certain embodiments, the detectable signal is optically visible and comprises a colorimetric dye that is detectable upon displacement.

In another aspect, disclosed herein is a method for detecting the presence of a phenethylamine-class compound in a sample, the method comprising providing the aptamer in the presence of the phenethylamine-class compound and measuring the output signal, thereby detecting the presence of the phenethylamine-class compound. Also disclosed herein, the sample may comprise a seized material or a biological fluid, wherein the biological fluid is selected from saliva, blood, serum, plasma, or urine. In some embodiments, the analyte is (+)-methamphetamine. In certain embodiments, the detectable signal is optically visible and comprises a colorimetric dye that is detectable upon displacement.

In a further aspect, disclosed herein is a screening assay for determining the presence of a phenethylamine-class compound in a sample, wherein the screening assay comprises an aptamer comprising a nucleic acid that specifically binds to the phenethylamine-class compound, wherein the aptamer exhibits less than 25% cross-reactivity to a structural analog or an enantiomer of the phenethylamine-class compound, and a detectable signal that is detectable upon binding of the nucleic acid to the phenethylamine-class compound. In some embodiments, the phenethylamine-class compound is methamphetamine. In some embodiments, the aptamer comprises a nucleic acid sequence as set forth in any one of SEQ ID NOS: 45, 46, 57, 64, 65, and 66, optionally comprising at least one additional mutation and/or at least one additional non-natural nucleic acid, wherein the mutation may comprise a thymine at a position corresponding to T18 or T39 that decreases affinity by at least 20-fold. In certain embodiments, the nucleic acid comprises a randomized region of at least 40 nucleotides that is thymine rich with at least 40 percent thymine content. In some embodiments, the detectable signal is optically visible and comprises a colorimetric dye detectable upon displacement.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows the top identified aptamer candidate. ML4 binds (+)-methamphetamine with a K_{D} of 2.5 μM and shows high selectivity to (+)-methamphetamine compared to structurally similar compounds.

FIGS. 2A-2E show affinity characterization of aptamers previously isolated for methamphetamine by other groups. FIG. 2A shows an aptamer previously identified by Ebrahimi et al. (Ebrahimi et. al. Systematic Evolution of Ligands by Exponential Enrichment Selection of Specific Aptamer for Sensing of Methamphetamine. Sens. Lett. 2013, 11 (3), 566-570), 84-nt aptaMETH and reported that this aptamer binds (+)-methamphetamine with a K_{D} of 100 nM. FIG. 2B shows isothermal calorimetry (ITC) results. These ITC results showed a far higher K_{D} of 364 μM in selection buffer of Ebrahimi et al. FIG. 2C shows ITC results of a 75-nt Aptamer-2, identified by Sester et al. (Sester et al. Unraveling the Binding Mode of a Methamphetamine Aptamer: A Spectroscopic and Calorimetric Study. Biophys. J. 2022, 121 (11), 2193-2205), with a reported K_{D} of 244 nM, but these ITC results again indicated a higher K_{D} of >1 mM for (+)-methamphetamine in the reported selection buffer. FIG. 2D shows the 82-nt Apta-4 aptamer found by Bor et al. which reportedly bound methamphetamine with a K_{D} of 1.3 μM. FIG. 2E shows ITC results indicating no binding of the 82-nt Apta-4 aptamer all in the reported selection buffer.

FIGS. 3A-3E show results of the first SELEX trial. FIG. 3A shows a simplified scheme of library-immobilized SELEX. FIG. 3B shows the aptamer initially hybridized to a biotinylated cDNA strand immobilized on agarose microbeads. Aptamer-target binding displaces the aptamer from the cDNA, releasing the aptamer into solution. FIG. 3C shows pool elution in each round of the first SELEX trial. FIG. 3D shows binding affinity of the Round 19 pool to (+)-methamphetamine determined using a gel-elution assay. The pool displayed no apparent affinity for the target. FIG. 3E shows sequencing enrichment results of round 13 and 19 pools. Round 13 and 19 pools were subjected to high-throughput sequencing (HTS). Enrichment-fold between Rounds 13 and 19 was plotted as a function of Round 19 abundance. Five top-ranked sequences (upper right) had abundance>0.08% and enrichment-fold>10. FIG. 3F shows the sequences of the five top-ranked sequences from FIG. 3E. None of these sequences displayed measurable affinity for (+)-methamphetamine based on ITC.

FIGS. 4A-4G show results of the second SELEX trial. FIG. 4A shows pool elution by target in each round of the second SELEX trial. FIG. 4B shows binding affinity of the Round 11 pool to (+)-methamphetamine as determined using a gel-elution assay. FIG. 4C shows enrichment-fold between Rounds 9 and 11 plotted as a function of Round 11 abundance. FIG. 4D shows the secondary structure of the most abundant aptamer discovered in this trial, MT2-R1. FIG. 4E shows the binding affinity of MT2-R1 to (+)-methamphetamine as determined by ITC. FIG. 4F shows the binding affinity of MT2-R1 to amphetamine determined using ITC. FIG. 4G shows the specificity of MT2-R1 and MT2-R2 toward several interferents using the exonuclease digestion assay. Heat-map indicates cross-reactivity relative to (+)-METH. The concentration of target and interferent was 250 μM, except for alprazolam, which was 50 μM due to solubility limits.

FIGS. 5A-5H show results for the third SELEX trial. FIG. 5A shows the N40 library used for library-immobilized SELEX. FIG. 5B shows pool elution by (+)-methamphetamine for each round of SELEX. FIG. 5C shows the binding affinity for (+)-methamphetamine as determined using the gel-elution assay for the Round 13, 15, and 18 pools. FIG. 5D shows the enrichment-fold of sequences between Rounds 13 and 18 plotted as a function of Round 18 abundance. Sequences with abundance>0.1% and enrichment-fold>2 are named and marked in blue. FIG. 5E shows the secondary structure of one of the highly enriched aptamers, M13, as predicted by NUPACK. FIG. 5F shows binding affinity of M13 to (+)-methamphetamine as determined using ITC. FIG. 5G shows binding affinity of M13 to amphetamine as determined using ITC. FIG. 5H shows the specificity of aptamers discovered in third trial to a panel of interferents as assessed via exonuclease digestion assay. Heat-map indicates cross-reactivity relative to 250 μM (+)-METH. The concentration of interferent employed was 250 M, but 100 μM for quinine and 50 μM for alprazolam due to solubility limitations.

FIGS. 6A-6H show results for the fourth SELEX trial. FIG. 6A shows the N40 library employed for library-immobilized SELEX. FIG. 6B shows pool elution by the target for each round of SELEX. FIG. 6C shows the binding affinity for (+)-methamphetamine based on a gel-elution assay for the Round 11 and 13 pools. FIG. 6D shows the enrichment-fold of sequences between Rounds 11 and 13 plotted as a function of Round 13 abundance. Sequences with abundance>0.1% and enrichment-fold>2 are named and marked in blue. FIG. 6E shows the secondary structure of aptamer ML4 as predicted by NUPACK. FIG. 6F shows the binding affinity of ML4 for (+)-methamphetamine as determined using ITC. FIG. 6G shows the binding affinity of ML4 for amphetamine as determined using ITC. FIG. 6H shows the specificity of aptamers discovered in fourth trial to a panel of interferents as assessed via exonuclease digestion assay. Heat-map indicates cross-reactivity relative to 500 μM (+)-methamphetamine. The concentration of interferent employed was 250 μM; alprazolam was 50 μM.

FIGS. 7A-7C show the identification of aptamer families and conserved sequence motifs from each trial of SELEX for (+)-methamphetamine via bioinformatic analysis. FIG. 7A shows families and motifs discovered in the final pool of the second SELEX trial. FIG. 7B shows families and motifs discovered in the final pool of the third SELEX trial. FIG. 7C shows families and motifs discovered in the final pool of the fourth SELEX trial. Plots in the middle represent the sequence space produced by Raptgen, with each dot representing a unique sequence. Aptamers close to each other in space are related to each other in sequence. Aptamer families in these plots are highlighted in red, and representative members are named. The primary motif in each family was determined using GLAM2, and a representative aptamer of that family is listed below the sequence logo along with its target-binding affinity and affinity for structurally related analogs of (+)-methamphetamine.

FIGS. 8A-8E show colorimetric detection of (+)-methamphetamine with an aptamer-based dye-displacement assay. FIG. 8A shows absorbance spectra of the cyanine dye X-732-91B dissolved in DMSO (left) and aqueous buffer (right) at concentrations of 0-10 μM. The structure of the dye is shown at the top center, and a photograph of solutions containing various concentrations of dye is shown at bottom center. FIG. 8B shows a scheme of the dye-displacement assay using aptamer ML4 and X-732-91B. Target binding displaces the dye from the aptamer into solution, causing the dye to aggregate and inducing a concomitant color change. FIG. 8C shows the calibration curve of colorimetric assay in both buffer (black) and 50% saliva (red). FIG. 8D shows the response of the assay to 0-6.4 μM (+)-methamphetamine. FIG. 8E shows assay cross-reactivity to 50 μM interferents. The red line demarcates 25% cross-reactivity relative to 25 μM (+)-methamphetamine.

FIG. 9 shows chemical structures of the compounds used in the experiment.

FIGS. 10A-10E show the binding affinity of aptamers discovered in the first trial of library-immobilized SELEX for (+)-methamphetamine based on ITC. FIG. 10A shows ITC results of (+)-methamphetamine titrated into MT1-R1. FIG. 10B shows ITC results of (+)-methamphetamine titrated into MT1-R2. FIG. 10C shows ITC results of (+)-methamphetamine titrated into MT1-R3. FIG. 10D shows ITC results of (+)-methamphetamine titrated into MT1-R4.

FIG. 10E shows ITC results of (+)-methamphetamine titrated into MT1-R5. Top panels display the heat generated from each titration of (+)-methamphetamine into MT1-R1, MT1-R2, MT1-R3, MT1-R4, and MT1-R5. Bottom panels show the integrated heat of each titration after correcting for the heat of dilution of the titrant.

FIGS. 11A-11D show the binding affinity of aptamers discovered in the second trial of library-immobilized SELEX to (+)-methamphetamine based on ITC. FIG. 11A shows ITC results of (+)-methamphetamine titrated into MT2-R1. FIG. 11B shows ITC results of (+)-methamphetamine titrated into MT2-R2. FIG. 11C shows ITC results of (+)-methamphetamine titrated into MT2-R3. FIG. 11D shows ITC results of (+)-methamphetamine titrated into MT2-R4. Top panels display the heat generated from each titration of (+)-methamphetamine into MT2-R1, MT2-R2, MT2-R3, and MT2-R4. Bottom panels show the integrated heat of each titration after correcting for the heat of dilution of the titrant.

FIGS. 12A-12B show the screening the specificity of two aptamer candidates (MT2-R1 and MT2-R2) from the second SELEX trial using the exonuclease digestion assay. FIG. 12A shows a plot showing resistance value obtained from time-course digestion of MT2-R1 in the presence of 250 μM selection target and various interferents, except for alprazolam (50 μM). FIG. 12B shows a plot showing resistance value obtained from time-course digestion of MT2-R2 in the presence of 250 μM selection target and various interferents, except for alprazolam (50 μM).

FIGS. 13A-13B show specificity of the enriched pools from the third trial of library-immobilized SELEX for (+)-methamphetamine as determined using the gel-elution assay. FIG. 13A shows pool elution by methamphetamine, each counter-target, and buffer alone (blue bar and red line) for Round 15. FIG. 13B shows pool elution by methamphetamine, each counter-target, and buffer alone (blue bar and red line) for Round 18.

FIGS. 14A-14H show the binding affinity of aptamers discovered in the third trial of library-immobilized SELEX to (+)-methamphetamine based on ITC. FIG. 14A shows ITC results of (+)-methamphetamine titrated into M1. FIG. 14B shows ITC results of (+)-methamphetamine titrated into M2. FIG. 14C shows ITC results of (+)-methamphetamine titrated into M3. FIG. 14D shows ITC results of (+)-methamphetamine titrated into M4. FIG. 14E shows ITC results of (+)-methamphetamine titrated into M5. FIG. 14F shows ITC results of (+)-methamphetamine titrated into M6. FIG. 14G shows ITC results of (+)-methamphetamine titrated into M7. FIG. 14H shows ITC results of (+)-methamphetamine titrated into M8. Top panels display the heat generated from each titration of (+)-methamphetamine into M1, M2, M3, M4, M5, M6, M7, and M8. Bottom panels show the integrated heat of each titration after correcting for the heat of dilution of the titrant.

FIGS. 15A-15H show the binding affinity of aptamers discovered in the third trial of library-immobilized SELEX for (+)-methamphetamine based on ITC. FIG. 15A shows ITC results of (+)-methamphetamine titrated into M9. FIG. 15B shows ITC results of (+)-methamphetamine titrated into M11. FIG. 15C shows ITC results of (+)-methamphetamine titrated into M12. FIG. 15D shows ITC results of (+)-methamphetamine titrated into M13. FIG. 15E shows ITC results of (+)-methamphetamine titrated into M14. FIG. 15F shows ITC results of (+)-methamphetamine titrated into M18. FIG. 15G shows ITC results of (+)-methamphetamine titrated into M20. FIG. 15H shows ITC results of (+)-methamphetamine titrated into M21. Top panels display the heat generated from each titration of (+)-methamphetamine into M9, M11, M12, M13, M14, M18, M20, and M21. Bottom panels show the integrated heat of each titration after correcting for the heat of dilution of the titrant.

FIGS. 16A-16I show the binding affinity of aptamers discovered in the third trial of library-immobilized SELEX for amphetamine based on ITC. FIG. 16A shows ITC results of (+)-methamphetamine titrated into M1. FIG. 16B shows ITC results of (+)-methamphetamine titrated into M2. FIG. 16C shows ITC results of (+)-methamphetamine titrated into M3. FIG. 16D shows ITC results of (+)-methamphetamine titrated into M4. FIG. 16E shows ITC results of (+)-methamphetamine titrated into M5. FIG. 16F shows ITC results of (+)-methamphetamine titrated into M6. FIG. 16G shows ITC results of (+)-methamphetamine titrated into M7. FIG. 16H shows ITC results of (+)-methamphetamine titrated into M8. FIG. 16I shows ITC results of (+)-methamphetamine titrated into M9. Top panels display the heat generated from each titration of amphetamine into M1, M2, M3, M4, M5, M6, M7, M8, and M9. Bottom panels show the integrated heat of each titration after correcting for the heat of dilution of the titrant.

FIGS. 17A-17G show the binding affinity of aptamers discovered in the third trial of library-immobilized SELEX for amphetamine based on ITC. FIG. 17A shows ITC results of (+)-methamphetamine titrated into M11. FIG. 17B shows ITC results of (+)-methamphetamine titrated into M12. FIG. 17C shows ITC results of (+)-methamphetamine titrated into M13. FIG. 17D shows ITC results of (+)-methamphetamine titrated into M14. FIG. 17E shows ITC results of (+)-methamphetamine titrated into M18. FIG. 17F shows ITC results of (+)-methamphetamine titrated into M20. FIG. 17G shows ITC results of (+)-methamphetamine titrated into M21. Top panels display the heat generated from each titration of amphetamine into M11, M12, M13, M14, M18, M20, and M21. Bottom panels show the integrated heat of each titration after correcting for the heat of dilution of the titrant.

FIGS. 18A-18E show screening the specificity of five aptamer candidates from the third SELEX trial using the exonuclease digestion assay. FIG. 18A shows a plot showing resistance value obtained from time-course digestion of M1 in the presence of 250 μM selection target and various interferents, except for alprazolam (50 μM). FIG. 18B shows a plot showing resistance value obtained from time-course digestion of M2 in the presence of 250 μM selection target and various interferents, except for alprazolam (50 μM). FIG. 18C shows a plot showing resistance value obtained from time-course digestion of M3 in the presence of 250 μM selection target and various interferents, except for alprazolam (50 μM). FIG. 18D shows a plot showing resistance value obtained from time-course digestion of M4 in the presence of 250 μM selection target and various interferents, except for alprazolam (50 μM). FIG. 18E shows a plot showing resistance value obtained from time-course digestion of M5 in the presence of 250 μM selection target and various interferents, except for alprazolam (50 μM).

FIGS. 19A-19E show screening of the specificity of five aptamer candidates from the third SELEX trial using the exonuclease digestion assay. FIG. 19A shows a plot showing resistance value obtained from time-course digestion of M6 in the presence of 250 μM selection target and various interferents, except for alprazolam (50 μM). FIG. 19B shows a plot showing resistance value obtained from time-course digestion of M7 in the presence of 250 μM selection target and various interferents, except for alprazolam (50 μM). FIG. 19C shows a plot showing resistance value obtained from time-course digestion of M8 in the presence of 250 μM selection target and various interferents, except for alprazolam (50 μM). FIG. 19D shows a plot showing resistance value obtained from time-course digestion of M9 in the presence of 250 μM selection target and various interferents, except for alprazolam (50 μM). FIG. 19E shows a plot showing resistance value obtained from time-course digestion of M11 in the presence of 250 μM selection target and various interferents, except for alprazolam (50 μM).

FIGS. 20A-20F show screening of the specificity of six aptamer candidates from the third SELEX trial using the exonuclease digestion assay. FIG. 20A shows a plot showing resistance value obtained from time-course digestion of M12 in the presence of 250 μM selection target and various interferents, except for alprazolam (50 μM). FIG. 20B shows a plot showing resistance value obtained from time-course digestion of M13 in the presence of 250 μM selection target and various interferents, except for alprazolam (50 μM). FIG. 20C shows a plot showing resistance value obtained from time-course digestion of M14 in the presence of 250 μM selection target and various interferents, except for alprazolam (50 μM). FIG. 20D shows a plot showing resistance value obtained from time-course digestion of M18 in the presence of 250 μM selection target and various interferents, except for alprazolam (50 μM). FIG. 20E shows a plot showing resistance value obtained from time-course digestion of M20 in the presence of 250 μM selection target and various interferents, except for alprazolam (50 μM). FIG. 20F shows a plot showing resistance value obtained from time-course digestion of M21 in the presence of 250 μM selection target and various interferents, except for alprazolam (50 μM).

FIGS. 21A-21B show the effect of including Triton X-100 in the selection buffer on washing efficiency during library-immobilized SELEX. FIG. 21A shows results of the naive library hybridized with biotinylated cDNA, immobilized onto streptavidin-coated agarose beads, and then washed with selection buffer with 0.005% Triton-X100, and then finally challenged with methamphetamine. Samples eluted from the column were collected and analyzed by PAGE. FIG. 21B shows results of the naive library hybridized with biotinylated cDNA, immobilized onto streptavidin-coated agarose beads, and then washed without selection buffer with 0.005% Triton-X100, and then finally challenged with methamphetamine. Samples eluted from the column were collected and analyzed by PAGE. ‘Ulib’=non-immobilized library after incubating library-cDNA complexes with streptavidin-coated agarose. ‘WB’=library washed away by ten washes with 250 μL selection buffer. ‘W1’=library eluted by one wash of 250 μL selection buffer prior to challenging the library with target. ‘Std’=a 10 nM standard made using the naïve library. ‘T1’, ‘T2’, and ‘T3’=elution of library after challenging with three sequential 250 μL aliquots of methamphetamine in selection buffer (without Triton X-100). ‘Beads’=library remaining on the beads at the end of the selection round.

FIG. 22 shows the specificity of the Round 13 pool from the fourth trial of library-immobilized SELEX for (+)-methamphetamine as determined using the gel-elution assay. Red line indicates the threshold of elution by the buffer-only negative control.

FIGS. 23A-23F show the binding affinity of aptamers isolated in the fourth trial of library-immobilized SELEX for (+)-methamphetamine based on ITC. FIG. 23A shows ITC results of (+)-methamphetamine titrated into ML1. FIG. 23B shows ITC results of (+)-methamphetamine titrated into ML2. FIG. 23C shows ITC results of (+)-methamphetamine titrated into ML3. FIG. 23D shows ITC results of (+)-methamphetamine titrated into ML4. FIG. 23E shows ITC results of (+)-methamphetamine titrated into ML5. FIG. 23F shows ITC results of (+)-methamphetamine titrated into ML6. Top panels display the heat generated from each titration of (+)-methamphetamine into ML1, ML2, ML3, ML4, ML5, and ML6. Bottom panels show the integrated heat of each titration after correcting for the heat of dilution of the titrant.

FIGS. 24A-24F show the binding affinity of aptamers isolated in the fourth trial of library-immobilized SELEX for (+)-methamphetamine based on ITC. FIG. 24A shows ITC results of (+)-methamphetamine titrated into ML7. FIG. 24B shows ITC results of (+)-methamphetamine titrated into ML8. FIG. 24C shows ITC results of (+)-methamphetamine titrated into ML10. FIG. 24D shows ITC results of (+)-methamphetamine titrated into ML11. FIG. 24E shows ITC results of (+)-methamphetamine titrated into ML12. FIG. 24F shows ITC results of (+)-methamphetamine titrated into ML13. Top panels display the heat generated from each titration of (+)-methamphetamine into ML7, ML8, ML10, ML11, ML12, and ML13. Bottom panels show the integrated heat of each titration after correcting for the heat of dilution of the titrant.

FIGS. 25A-25F show the binding affinity of aptamers discovered in the fourth trial of library-immobilized SELEX for amphetamine based on ITC. FIG. 25A shows ITC results of amphetamine titrated into ML1. FIG. 25B shows ITC results of amphetamine titrated into ML2. FIG. 25C shows ITC results of amphetamine titrated into ML3. FIG. 25D shows ITC results of amphetamine titrated into ML4. FIG. 25E shows ITC results of amphetamine titrated into ML5. FIG. 25F shows ITC results of amphetamine titrated into ML6. Top panels display the heat generated from each titration of amphetamine into ML1, ML2, ML3, ML4, ML5, and ML6. Bottom panels show the integrated heat of each titration after correcting for the heat of dilution of the titrant.

FIGS. 26A-26F show the binding affinity of aptamers discovered in the fourth trial of library-immobilized SELEX for amphetamine based on ITC. FIG. 26A shows ITC results of amphetamine titrated into ML7. FIG. 26B shows ITC results of amphetamine titrated into ML8. FIG. 26C shows ITC results of amphetamine titrated into ML10. FIG. 26D shows ITC results of amphetamine titrated into ML11. FIG. 26E shows ITC results of amphetamine titrated into ML12. FIG. 26F shows ITC results of amphetamine titrated into ML13. Top panels display the heat generated from each titration of amphetamine into ML7, ML8, ML10, ML11, ML12, and ML13. Bottom panels show the integrated heat of each titration after correcting for the heat of dilution of the titrant.

FIGS. 27A-27E show screening of the specificity of five aptamer candidates from the fourth SELEX trial using the exonuclease digestion assay. FIG. 27A shows a plot showing resistance values obtained from time-course digestion of ML1 in the presence of 250 μM selection target and various interferents, except for alprazolam (50 μM). FIG. 27B shows a plot showing resistance values obtained from time-course digestion of ML2 in the presence of 250 μM selection target and various interferents, except for alprazolam (50 μM). FIG. 27C shows a plot showing resistances value obtained from time-course digestion of ML3 in the presence of 250 μM selection target and various interferents, except for alprazolam (50 μM). FIG. 27D shows a plot showing resistances value obtained from time-course digestion of ML4 in the presence of 250 μM selection target and various interferents, except for alprazolam (50 μM). FIG. 27E shows a plot showing resistance values obtained from time-course digestion of ML5 in the presence of 250 μM selection target and various interferents, except for alprazolam (50 μM).

FIGS. 28A-28E shows screening of the specificity of five aptamer candidates from the fourth SELEX trial using our exonuclease digestion assay. FIG. 28A shows a plot showing resistance values obtained from time-course digestion of ML6 in the presence of 250 μM selection target and various interferents, except for alprazolam (50 μM). FIG. 28B shows a plot showing resistance values obtained from time-course digestion of ML7 in the presence of 250 μM selection target and various interferents, except for alprazolam (50 μM). FIG. 28C shows a plot showing resistance values obtained from time-course digestion of ML10 in the presence of 250 μM selection target and various interferents, except for alprazolam (50 μM). FIG. 28D shows a plot showing resistance values obtained from time-course digestion of ML11 in the presence of 250 μM selection target and various interferents, except for alprazolam (50 μM). FIG. 28E shows a plot showing resistance values obtained from time-course digestion of ML13 in the presence of 250 μM selection target and various interferents, except for alprazolam (50 μM).

FIGS. 29A-29F show the binding affinity of aptamers for (+)-methamphetamine based on ITC. FIG. 29A shows ITC results of (+)-methamphetamine titrated into MU in the presence of 1 mm Mg²⁺. FIG. 29B shows ITC results of (+)-methamphetamine titrated into MU in the presence of 2 mm Mg²⁺. FIG. 29C shows ITC results of (+)-methamphetamine titrated into ML3 in the presence of 5 mm Mg²⁺. FIG. 29D shows ITC results of (+)-methamphetamine titrated into ML4 in the presence of 1 mm Mg²⁺. FIG. 29E shows ITC results of (+)-methamphetamine titrated into ML4 in the presence of 2 mm Mg²⁺. FIG. 29F shows ITC results of (+)-methamphetamine titrated into ML4 in the presence of 5 mm Mg²⁺. Top panels display the heat generated from each titration of (+)-methamphetamine into ML3 and ML4 in the presence of 1, 2 and 5 mM Mg²⁺, respectively. Bottom panels show the integrated heat of each titration after correcting for the heat of titrant dilution.

FIGS. 30A-30B show the binding affinity of MLM, an aptamer discovered in the fourth trial of library-immobilized SELEX, for interferents based on ITC. FIG. 30A shows ITC results of 4-HMA titrated into MLM. FIG. 30B shows ITC results of (−)-METH titrated into ML4. Top panels display the heat generated from each titration of MLM into 4-HMA and (−)-METH. Bottom panels show the integrated heat of each titration after correcting for the heat of dilution of the titrant.

FIGS. 31A-31D show the binding affinity of MLM, an aptamer discovered in the fourth trial of library-immobilized SELEX, for interferents based on ITC. FIG. 31A shows ITC results of MLM titrated into methylphenidate. FIG. 31B shows ITC results of MLM titrated into (+)-pseudoephedrine. FIG. 31C shows ITC results of MLM titrated into MDMA. FIG. 31D shows ITC results of MLM titrated into MDPV. Top panels display the heat generated from each titration of MLM into methylphenidate, (+)-pseudoephedrine, MDMA, and MDPV. Bottom panels show the integrated heat of each titration after correcting for the heat of dilution of the titrant.

FIG. 32 shows the secondary structures of methamphetamine aptamers obtained from different SELEX trials predicted by NUPACK.

FIGS. 33A-33H show the mutational analysis of ML3. FIG. 33A shows the design of six point-mutants derived from ML3. FIG. 33B shows resistance values of ML3 and six mutants determined by T5 exonuclease/exonuclease I digestion assay. FIG. 33C shows ITC results of (+)-methamphetamine titrated into ML3-mut1. FIG. 33D shows ITC results of (+)-methamphetamine titrated into ML3-mut2. FIG. 33E shows ITC results of (+)-methamphetamine titrated into ML3-mut3. FIG. 33F shows ITC results of (+)-methamphetamine titrated into ML3-mut4. FIG. 33G shows ITC results of (+)-methamphetamine titrated into ML3-mut5. FIG. 33H shows ITC results of (+)-methamphetamine titrated into ML3-mut6. Top panels display the heat generated from each titration of (+)-methamphetamine into ML3-mut1, ML3-mut2, ML3-mut3, ML3-mut4, ML3-mut5, and ML3-mut6. Bottom panels show the integrated heat of each titration after correcting for the heat of titrant dilution.

FIGS. 34A-34I show mutational analysis of ML4. FIG. 34A shows the design of seven point-mutants derived from ML4. FIG. 34B shows the resistance values of ML4 and seven mutants determined by T5 exonuclease/exonuclease I digestion assay. FIG. 34C shows the binding affinity of ML4-mut1 for (+)-methamphetamine based on ITC. FIG. 34D shows the binding affinity of ML4-mut2 for (+)-methamphetamine based on ITC. FIG. 34E shows the binding affinity of ML4-mut3 for (+)-methamphetamine based on ITC. FIG. 34F shows the binding affinity of ML4-mut4 for (+)-methamphetamine based on ITC. FIG. 34G shows the binding affinity of ML4-mut5 for (+)-methamphetamine based on ITC. FIG. 34H shows the binding affinity of ML4-mut6 for (+)-methamphetamine based on ITC. FIG. 34I shows the binding affinity of ML4-mut7 for (+)-methamphetamine based on ITC. Top panels display the heat generated from each titration of (+)-methamphetamine into ML4-mut1, ML4-mut2, ML4-mut3, ML4-mut4, ML4-mut5, ML4-mut6, and ML4-mut7. Bottom panels show the integrated heat of each titration after correcting for the heat of titrant dilution.

FIGS. 35A-35B show the binding of X-732-91B to aptamer MLM. FIG. 35A shows the absorbance spectra of solutions containing 4 μM X-732-91B with 0, 2, 4, 6, 8 or 10 μM MLM, where the black-to-red color gradient shows increasing aptamer concentrations. FIG. 35B shows absorbance of X-732-91B monomers at 568 nm and H-aggregates at 450 nm plotted against the concentration of MLM.

FIGS. 36A-36B show the effect of (+)-methamphetamine on the absorbance spectrum of X-732-91B alone. FIG. 36A shows the spectra of 4 μM X-732-91B in the presence of 0, 0.39, 0.78, 1.56, 3.13, 6.25, 12.5, 25, 50, 100, and 200 μM (+)-methamphetamine with no aptamer present, where the black-to-red color gradient represents increasing target concentrations. FIG. 36B shows the peak absorbance at 450 (black) and 568 (red) nm as a function of (+)-methamphetamine concentration.

FIGS. 37A-37B show the detection of (+)-methamphetamine using X-732-91B and aptamer ML4. FIG. 37A shows the absorbance spectra in buffer for samples containing 4 μM X-732-91B and 6 μM ML4 in the presence of 0, 0.39, 0.78, 1.56, 3.13, 6.25, 12.5, 25, 50, 100, and 200 μM (+)-methamphetamine, where the black-to-red color gradient represents increasing target concentrations. FIG. 37B shows the absorbance spectra in 50% saliva for samples containing 4 μM X-732-91B and 6 μM ML4 in the presence of 0, 0.39, 0.78, 1.56, 3.13, 6.25, 12.5, 25, 50, 100, and 200 μM (+)-methamphetamine, where the black-to-red color gradient represents increasing target concentrations.

FIG. 38 shows the specificity of a dye-displacement assay for (+)-methamphetamine based on aptamer MLM and dye X-732-91B. Photographs were taken after samples containing dye without aptamer (−ML4) or dye-aptamer complex (+ML4) were challenged with (+)-methamphetamine or various interferents for 10 minutes.

FIGS. 39A-39B show the detection of (+)-methamphetamine in a mixture of interferents using an aptamer-based dye-displacement assay. FIG. 39A shows absorbance spectra in buffer for samples containing 4 μM X-732-91B and 6 μM aptamer ML4 in the presence of 2.5 and 5 μM (+)-methamphetamine alone or with a mixture of 1 μM morphine, 3 μM cocaine, 2 μM methadone, and 1 μM fentanyl. FIG. 39B shows the signal gain which was calculated as (R−R₀)/R₀, where R and R₀ are the ratio of the area under the curve (AUC) for dye aggregates (400-505 nm) relative to the AUC for monomers (505-620 nm) with and without target/interferent, respectively.

FIG. 40 shows the specificity of a dye-displacement assay for (+)-methamphetamine based on aptamer MLM and dye X-732-91B. The plot shows signal gain and assay cross-reactivity to clinically relevant target concentrations as well as much higher concentrations of various interferents. The red line demarcates 25% cross-reactivity relative to 2.5 μM (+)-methamphetamine.

FIGS. 41A-41B show the scheme of electrochemical aptamer-based (EAB) sensors for (+)-methamphetamine detection. FIG. 41A shows the predicted structures of aptamer sequences employed. FIG. 41B shows the working principle of (+)-methamphetamine EAB sensing.

FIGS. 42A-42B show EAB sensing performance of LI3-45-MB. FIG. 42A shows Calibration curves of 0-1.75 mM (+)-methamphetamine in buffer. FIG. 42B shows the cross reactivity of EAB against various counter non-target molecules.

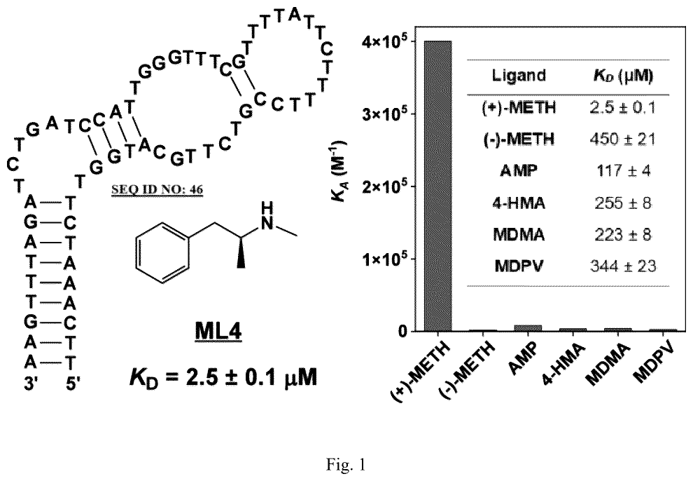

### DETAILED DESCRIPTION

It is appreciated that certain features of the disclosure, which are, for clarity, described in the context of separate aspects, can also be provided in combination with a single aspect. Conversely, various features of the disclosure, which are, for brevity, described in the context of a single aspect, can also be provided separately or in any suitable sub combination. Unless defined otherwise, all technical and scientific terms used herein have the same meaning as commonly understood by one of ordinary skill in the art. Methods and materials similar or equivalent to those described herein can be used in the practice or testing of the present disclosure.

### Definitions

In this specification and in the claims that follow, reference will be made to a number of terms, which shall be defined to have the following meanings:

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a compound” or “a composition” includes, but is not limited to, two or more such compounds, compositions, or cancers, and the like.

It should be noted that ratios, concentrations, amounts, and other numerical data can be expressed herein in a range format. It can be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it can be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less' and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

Reference is made herein to nucleic acid and nucleic acid sequences. The terms “nucleic acid” and “nucleic acid sequence” refer to a nucleotide, oligonucleotide, polynucleotide (which terms may be used interchangeably), or any fragment thereof. These phrases also refer to DNA or RNA of genomic or synthetic origin (which may be single-stranded or double-stranded and may represent the sense or the antisense strand).

Variants comprising deletions relative to a reference amino acid sequence or nucleotide sequence are contemplated herein. A “deletion” refers to a change in the amino acid or nucleotide sequence that results in the absence of one or more amino acid residues or nucleotides relative to a reference sequence. A deletion removes at least 1, 2, 3, 4, 5, 10, 20, 50, 100, or 200 amino acids residues or nucleotides. A deletion may include an internal deletion or a terminal deletion (e.g., an N-terminal truncation or a C-terminal truncation or both of a reference polypeptide or a 5′-terminal or 3′-terminal truncation or both of a reference polynucleotide).

Variants comprising a fragment of a reference amino acid sequence or nucleotide sequence are contemplated herein. A “fragment” is a portion of an amino acid sequence or a nucleotide sequence which is identical in sequence to but shorter in length than the reference sequence. A fragment may comprise up to the entire length of the reference sequence, minus at least one nucleotide/amino acid residue. For example, a fragment may comprise from 5 to 1000 contiguous nucleotides or contiguous amino acid residues of a reference polynucleotide or reference polypeptide, respectively. In some embodiments, a fragment may comprise at least 5, 10, 15, 20, 21, 22, 23, 24, 25, 26, 27, 28, 29, 30, 40, 50, 60, 70, 80, 90, 100, 150, 250, or 500 contiguous nucleotides or contiguous amino acid residues of a reference polynucleotide or reference polypeptide, respectively. Fragments may be preferentially selected from certain regions of a molecule, for example the N-terminal region and/or the C-terminal region of a polypeptide or the 5′-terminal region and/or the 3′ terminal region of a polynucleotide. The term “at least a fragment” encompasses the full length polynucleotide or full length polypeptide.

Variants comprising insertions or additions relative to a reference sequence are contemplated herein. The words “insertion” and “addition” refer to changes in an amino acid or nucleotide sequence resulting in the addition of one or more amino acid residues or nucleotides. An insertion or addition may refer to 1, 2, 3, 4, 5, 10, 20, 30, 40, 50, 60, 70, 80, 90, 100, 150, or 200 amino acid residues or nucleotides.

The terms “percent identity” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity for a nucleic acid sequence may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at the NCBI website. The “BLAST 2 Sequences” tool can be used for both blastn and blastp (discussed above).

Percent identity may be measured over the length of an entire defined polynucleotide sequence or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length may be used to describe a length over which percentage identity may be measured.

A “full length” polynucleotide sequence is one containing at least a translation initiation codon (e.g., methionine) followed by an open reading frame and a translation termination codon. A “full length” polynucleotide sequence encodes a “full length” polypeptide sequence.

A “variant,” “mutant,” or “derivative” of a particular nucleic acid sequence may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). In some embodiments a variant polynucleotide aptamer may show, for example, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length relative to a reference polynucleotide aptamer.

Nucleic acid sequences that do not show a high degree of identity may nevertheless encode similar amino acid sequences due to the degeneracy of the genetic code. It is understood that changes in a nucleic acid sequence can be made using this degeneracy to produce multiple nucleic acid sequences that all encode substantially the same protein.

“Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame.

A “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques such as those described in Sambrook, J. et al. (1989) Molecular Cloning: A Laboratory Manual, 2nd ed., vol. 1 3, Cold Spring Harbor Press, Plainview N.Y. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

“Substantially isolated or purified” nucleic acid or amino acid sequences are contemplated herein. The term “substantially isolated or purified” refers to nucleic acid or amino acid sequences that are removed from their natural environment, and are at least 60% free, preferably at least 75% free, and more preferably at least 90% free, even more preferably at least 95% free from other components with which they are naturally associated.

The term “mismatched” or “mismatched target sequence” refers to an off-target sequence that is not perfectly complementary to the first DNA sequence or the second DNA sequence of the chimeric deoxyribonucleic acid described herein. The dual retargeted DNA may have at least one mismatch, but can also have 2, 3, 4, 5, 6 or 7 or more mismatched nucleotides to the off-target sequence.

As used herein, the term “detecting,” in the context of measuring a signal from a detectable output to indicate the presence of a target analyte such as a phenethylamine-class compound in a sample, does not require that the method achieve 100% sensitivity or 100% specificity. As is well understood in the art, “sensitivity” refers to the probability that the assay yields a positive result when the target analyte is present in the sample, while “specificity” refers to the probability that the assay yields a negative result when the target analyte is absent. In certain embodiments, a sensitivity of at least 50% is acceptable, with sensitivities of at least 60%, 70%, 75%, 80%, 90%, or 99% being increasingly preferred. Likewise, a specificity of at least 50% is acceptable, with specificities of at least 60%, 70%, 80%, 90%, or 99% being increasingly preferred. Specificity can also be defined by lack of cross-reactivity, which is discussed in more detail below.

As used herein, the term “aptamer” refers to a non-naturally occurring nucleic acid that exhibits a desirable functional interaction with a target molecule, such as a phenethylamine-class compound. Desirable interactions include, but are not limited to, binding the target with high specificity and affinity, catalytically modifying the target, altering the target's structure or activity, covalently attaching to the target, or facilitating a reaction between the target and another molecule. In certain embodiments, the interaction is specific binding to a target analyte that is a low epitope small molecule distinct from polynucleotides, wherein the binding occurs through a mechanism independent of Watson-Crick base pairing or triple helix formation. The aptamer can be a non-naturally occurring nucleic acid whose known physiological function involves binding to the target molecule. Aptamers to a given phenethylamine-class compound can be obtained from a candidate mixture of nucleic acids using a selection workflow that includes the methods disclosed herein.

“Specific binding affinity” means the aptamer binds its target analyte with a substantially greater affinity than it binds to non-target molecules, including structural analogs and endogenous interferents that may be present in complex samples. For example, the aptamer can exhibit less than 50%, 45%, 40%, 35%, 30%, 25%, 20%, 15%, 10%, 5%, or 1% cross-reactivity to a structural analog or an enantiomer of the target, such as the phenethylamine-class compound.

An aptamer as described herein may contain any suitable number of nucleotides, may be composed of DNA, RNA, or combinations thereof, and may be single-stranded, double-stranded, or contain mixed double-stranded or triple-stranded regions. The aptamer may also include chemically modified bases, non-natural nucleotides, or backbone modifications, and may incorporate sequence motifs such as randomized regions or thymine-rich segments that contribute to target recognition and signal generation in the disclosed detection assays. The term “aptamers” refers to one or more such molecules, which may vary in length, sequence, and modification profile while maintaining the claimed binding properties.

Although the “low complexity analytes/low epitope targets/small-molecules,” described herein include phenethylamine-class compounds such as methamphetamine, the aptamer-dye complexes may also be configured to detect other analytes with similar structural or chemical features. Examples include other small-molecule amines such as tyramine, histamine, serotonin, or nicotine, as well as pharmaceutical agents including ephedrine, phenylephrine, or propranolol. In some embodiments, the aptamer can be adapted to detect pesticide residues, veterinary drugs, or environmental contaminants with similar physicochemical properties.

As used herein, a buffer refers to an aqueous solution that resists changes in pH upon addition of an acid or base and provides ionic conditions suitable for aptamer stability and binding activity. Buffers may include, but are not limited to, phosphate-buffered saline (PBS), Tris(hydroxymethyl)aminomethane (Tris) buffer, HEPES buffer, MOPS buffer, and citrate buffer. In some embodiments, the buffer may further contain one or more salts, such as sodium chloride, potassium chloride, or ammonium acetate, to adjust ionic strength. In certain embodiments, the buffer may include divalent cations such as magnesium ions (Mg²⁺), calcium ions (Ca²⁺), manganese ions (Mn²⁺), or zinc ions (Zn²⁺) to stabilize nucleic acid folding or facilitate analyte binding. The pH of the buffer may range from about 5.5 to about 9.0, including about 6.5, 7.0, 7.4, or 8.0, depending on the intended assay conditions. In some embodiments, the buffer may include additives such as bovine serum albumin (BSA), Tween-20, or polyethylene glycol (PEG) to reduce non-specific binding.

As used herein, “small molecule analyte” refers to an organic or organometallic compound typically below 1,000 Da, more typically below 500 Da, that lacks repeating peptide or nucleic acid backbones.

As used herein, “low epitope small molecule analyte” refers to a small molecule that presents one or two dominant recognition features under the selection conditions, for example a single basic nitrogen and an aromatic ring, or a single carboxyl group.

As used herein, “phenethylamine class compound” refers to a molecule comprising a benzene ring linked through a two carbon ethyl chain to a primary or secondary amine.

As used herein, “structural analog” refers to a compound that is structurally related to the analyte and is reasonably expected to compete or interfere in an assay.

As used herein, “library immobilized SELEX” refers to a selection workflow in which a nucleic acid library is immobilized on a solid support through a complementary capture strand, the target is presented in solution, and sequences released upon target binding are collected and amplified.

As used herein, “counter selection” refers to a selection step that exposes the library or enriched pool to one or more non target species to deplete sequences that bind the non target.

As used herein, “randomized region length” refers to the number of contiguous, initially random nucleotides in a nucleic acid library.

As used herein, “divalent cation” refers to a doubly charged cation present in selection or assay buffers that can influence nucleic acid folding and target binding, including magnesium and calcium.

As used herein, “complex sample” or “Biological matrix” refers to a sample containing endogenous constituents that may affect assay performance, including saliva, serum, plasma, urine, whole blood, seized material extracts, food extracts, and wastewater.

As used herein, “application-relevant conditions” refers to buffer composition, ionic strength, pH, temperature, and matrix components representative of intended use, for example saliva at physiological ionic strength and pH.

As used herein, “binding affinity” refers to the strength of the interaction between the aptamer and the analyte, commonly expressed as a dissociation constant K_{D} measured in solution, with lower K_{D} indicating tighter binding.

As used herein, “specificity” refers to the qualitative ability of a nucleic acid aptamer to recognize its intended analyte over unrelated classes of molecules under defined assay conditions (including buffer composition, pH, ionic strength, temperature, and matrix content). Specificity is established by the absence of detectable binding or signal to a representative panel of unrelated species tested at equal or higher molar concentration than the analyte. In certain embodiments, no false-positive binding or signal is observed for any unrelated species at 21× the analyte test concentration; in preferred embodiments, at ≥5×; and in more preferred embodiments, at ≥10× the analyte test concentration.

As used herein, “selectivity” refers to the quantitative ability of an aptamer or assay to distinguish the analyte from defined structural analogs or interferents, expressed as a fold discrimination ratio based on binding or signal. For binding-based selectivity, the ratio is defined as K_{D}(analog)/K_{D}(analyte), where larger values indicate better selectivity; for signal-based selectivity at equal molarity, the ratio is Signal(analyte)/Signal(analog), where larger values likewise indicate better selectivity. Unless stated otherwise, selectivity values are reported as the worst-case (lowest) ratio across a predefined list of relevant interferents at operational concentrations. For small-molecule targets, minimum, preferred, and more preferred selectivity ratios are ≥10×, ≥50×, and ≥100-500×, respectively; for peptide or protein targets, minimum, preferred, and more preferred ratios are ≥20×, ≥100×, and ≥300-1,000×, respectively; and for closely related ions or very near structural analogs, minimum, preferred, and more preferred ratios are ≥5×, ≥20×, and ≥50-100×, respectively.

As used herein, “robustness” refers to the ability of an assay or aptamer to maintain predefined performance metrics—such as K_{D} or EC₅₀, limit of detection (LOD) or limit of quantitation (LOQ), dynamic range or slope, baseline signal, precision (% CV), and on-target selectivity-across expected variations in sample matrix, interferents, ionic strength, pH, temperature, user handling, and manufacturing lots. In certain embodiments, performance is maintained within the following windows: pH 6.5-8.5 (preferably 7.2-7.8); NaCl 50-500 mM and Mg²+0-10 mM; use temperature 15-37° C. with storage between 4-25° C.; and matrix content from 0-50% biological fluid or other relevant matrix.

As used herein, “fold discrimination” refers to a ratio quantifying selectivity, defined as the value of a chosen performance metric for the analyte divided by the corresponding value for a comparator species under identical conditions. Exemplary metrics include (i) K_{D}-based: K_{D}(comparator)/K_{D}(analyte); (ii) signal-based at equal molarity: Signal(analyte)/Signal(comparator); (iii) concentration at equal response (e.g., EC50-based): EC50(comparator)/EC50(analyte); and (iv) detection limits: LOD(comparator)/LOD(analyte), where larger values indicate better discrimination. Unless indicated otherwise, acceptable fold discrimination is at least 5-10× for closely related analogs and at least 10-20× for typical interferent panels, with preferred values≥50× and more preferred values 2100-1,000×, depending on target class and application.

As used herein, “isothermal titration calorimetry” or “ITC” refers to a solution phase biophysical method that measures heat changes upon binding to determine thermodynamic parameters such as K_{D}, enthalpy, and stoichiometry.

As used herein, “exonuclease protection assay” refers to an assay in which target binding protects an aptamer from exonuclease digestion, producing a measurable preservation of nucleic acid that correlates with binding in solution.

As used herein, “dye displacement assay” refers to a colorimetric assay in which a reporter dye bound to the aptamer is displaced upon analyte binding, generating a visible or spectrophotometric change proportional to analyte concentration.

As used herein, “limit of detection” or “LOD” refers to the lowest analyte concentration that produces a signal statistically distinguishable from a blank under stated conditions.

As used herein, “limit of quantitation” or “LOQ” refers to the lowest analyte concentration that can be quantified with acceptable precision and accuracy under stated conditions.

As used herein, “dynamic range” refers to the analyte concentration interval over which the assay response is monotonic and meets predefined accuracy and precision specifications.

As used herein, “enrichment round” refers to one complete cycle of selection that includes binding, partitioning, recovery of binders, and amplification.

As used herein, “field-deployable test” refers to a test that operates without complex instrumentation, provides a user perceptible output within a short time, and tolerates storage and handling conditions typical of field settings.

As used herein, “fold change” refers to the ratio of the absorbance measured in the presence of the analyte to the absorbance measured in a reference or control condition (such as without the analyte), representing how many times higher or lower the optical signal is due to analyte binding. The fold value describes the relative change in measurable colorimetric output caused by analyte binding. The “stated fold discrimination” refers to how much stronger the signal is for the target analyte compared to signals from structural analogs or interfering compounds.

$$Fold Change = \frac{Signal with Analyte}{Signal without Analyte ( or with Control )}$$

where “signal” could be an absorbance reading at a specific wavelength, optical density, or any other quantifiable colorimetric output.
To measure specificity, the analyte fold change is compared to that of a structural analog under identical assay conditions:

$$Fold Discrimination = \frac{Fold Change for Target Analyte}{Fold Change for Non - Target Structural Analog}$$

### Aptamers

Aptamers are short oligonucleotide-based affinity reagents that are increasingly being used in biosensors for applications including diagnostics and biomedical research. Selection techniques such as library-immobilized systematic evolution of ligands by exponential enrichment (SELEX) have made it feasible to isolate aptamers for small-molecule targets, but, until the present invention, it remained challenging to generate aptamers with high affinity and specificity for targets with few functional groups to facilitate recognition by nucleic acids.

To address this challenge, strategies have been discovered for optimizing the isolation of high-performance aptamers for phenethylamine-class compounds, such as (+)-methamphetamine, a target for which previously reported aptamers have extremely weak or no binding affinity. This method of discovery is described in more detail below. The method yielded high-quality aptamers containing longer conserved motifs than those typically used with aptamers. These conserved motifs are more informationally dense than aptamers with mediocre affinity and poor specificity.

The aptamers disclosed herein can rapidly detect phenethylamine-class compounds, such as (+)-methamphetamine, at toxicologically relevant concentrations in saliva in a colorimetric dye-displacement assay. These aptamers, and the methods of generating them, show the feasibility of generating high-quality aptamers for challenging, low-complexity small-molecule targets.

Specifically, disclosed herein is an aptamer comprising (a) a nucleic acid that specifically binds to a phenethylamine-class compound, wherein the aptamer exhibits less than 25% cross-reactivity to a structural analog or an enantiomer of the phenethylamine-class compound; and (b) a detectable signal, wherein the signal is detectable upon binding of the nucleic acid to the phenethylamine-class compound.

As used herein, the “low epitope small molecule”, “low complexity analyte”, “low complexity target”, or “low complexity small molecule” refers to a small molecule with at least one or more dominant recognition elements under the selection conditions. The low epitope small molecule typically comprises a molecular weight of about 250 Da or less. The low epitope small molecule comprises at least one functional group available for recognition by the aptamer. A specific example of such a molecule is phenethylamine-class compounds, such as methamphetamine.

In other embodiments, the low epitope small molecule is selected from a primary aliphatic amine, a primary benzylic amine, a secondary aliphatic amine or a secondary benzylic amine (including but not limited to, such as, for example, phenethylamine, amphetamine, methamphetamine, 1 phenyl 2 propylamine, 2 phenyl propylamine, benzylamine, ethylamine, propylamine, isopropylamine, butylamine, cyclohexylamine, aniline, p toluidine, m toluidine, or o toluidine); phenethylamine analogs (including but not limited to, such as, for example, amphetamine, methamphetamine, 3,4 methylenedioxyamphetamine, 3,4 methylenedioxymethamphetamine, phenethylamine, cathinone, methcathinone, ephedrine, pseudoephedrine, phenylpropanolamine); zwitterionic amino compounds or small neurotransmitters (including but not limited to, such as, for example, gamma aminobutyric acid, beta alanine, glycine, taurine, ethanolamine, sarcosine, betaine, creatine); small carboxylates or small acids (including but not limited to, such as, for example, acetic acid, propionic acid, butyric acid, valeric acid, benzoic acid, salicylic acid, lactic acid, pyruvic acid, succinic acid); small alcohols or diols (including but not limited to, such as, for example, methanol, ethanol, 1 propanol, 2 propanol, 1 butanol, 2 butanol, tert butanol, ethylene glycol, propylene glycol); simple ketones and aldehydes (including but not limited to, such as, for example, acetone, methyl ethyl ketone, methyl isobutyl ketone, formaldehyde, acetaldehyde, propionaldehyde, benzaldehyde); simple aromatic compounds with a single substituent (including but not limited to, such as, for example, toluene, ethylbenzene, styrene, phenol, anisole, benzonitrile, nitrobenzene, chlorobenzene); small heterocycles with a single dominant functionality (including but not limited to, such as, for example, pyridine, piperidine, morpholine, imidazole, pyrrolidine, thiazole, oxazole); small quaternary ammonium and related cations (including but not limited to, such as, for example, choline, acetylcholine, carnitine, tetramethylammonium); small organic bases and amides (including but not limited to, such as, for example, urea, acetamide, formamide, dimethylformamide, acetonitrile).

In some embodiments, the low epitope small molecule is selected from the group consisting of primary or secondary aliphatic amines, benzylic amines, zwitterionic amino compounds, small carboxylates, small alcohols, simple ketones, simple aldehydes, single substituted aromatics, small heterocycles, quaternary ammonium species, and small organic bases or amides.

When the small molecule is a phenethylamine class compound, it can be selected from amphetamine, methamphetamine, phenethylamine, 3,4 methylenedioxyamphetamine, and 3,4 methylenedioxymethamphetamine.

In some embodiments, selectivity is established against a panel that includes amphetamine, 3,4 methylenedioxymethamphetamine, 3,4 methylenedioxyamphetamine, cathinone, ephedrine, pseudoephedrine, phenylpropanolamine, and phenethylamine.

In some embodiments, robustness is established in saliva or serum in the presence of endogenous small molecules selected from gamma aminobutyric acid, glycine, taurine, lactic acid, acetic acid, urea, creatine, choline, and ethanolamine.

In some embodiments, the low epitope small molecule comprises an aromatic ring and a single basic nitrogen or a single carboxyl group, with no more than one additional heteroatom substituent.

In some aspects, the nucleic acid aptamer can include DNA. In other aspects, the nucleic acid aptamer can include RNA. In yet other aspects, the nucleic acid aptamer can include a combination of DNA, RNA, and modified nucleic acids.

In some embodiments, the nucleic acid aptamer includes at least one mutation and/or at least one non-natural nucleic acid. As used herein, “non-natural nucleic acid” refers to any synthetic or chemically modified nucleotide, such as 2′-O-methyl nucleotides, locked nucleic acids (LNA), peptide nucleic acids (PNA), phosphorothioate backbones, or nucleotides containing modified bases like 5-methylcytosine. In certain embodiments, a thymine mutation at a position corresponding to T18 or T39 reduces binding affinity for the low complexity analyte by at least 20-fold, 50-fold, or even 100-fold, as determined by changes in the dissociation constant (K_{D}).

In some aspects, the nucleic acid aptamer can include 80% similarity or more (e.g., 81% similarity or more, 82% similarity or more, 83% similarity or more, 84% similarity or more, 85% similarity or more, 86% similarity or more, 87% similarity or more, 88% similarity or more, 89% similarity or more, 90% similarity or more, 91% similarity or more, 92% similarity or more, 93% similarity or more, 94% similarity or more, 95% similarity or more, 96% similarity or more, 97% similarity or more, 98% similarity or more, 99% similarity or more) to any one of SEQ ID NOS: 45, 46, 57, 64, 65, and 66. In some aspects, the nucleic acid aptamer can include any one of SEQ ID NOS: 45, 46, 57, 64, 65, and 66. These sequences are given in Tables 9 and 10.

In some aspects, the nucleic acid aptamer can be at least about 40 nucleotides (e.g., at least about 45 nucleotides, at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides, at least about 100 nucleotides, at least about 105 nucleotides, at least about 110 nucleotides, at least about 115 nucleotides, at least about 120 nucleotides) in length. In some aspects, the nucleic acid aptamer can be up to about 120 nucleotides (e.g., up to about 115 nucleotides, up to about 110 nucleotides, up to about 105 nucleotides, up to about 100 nucleotides, up to about 95 nucleotides, up to about 90 nucleotides, up to about 85 nucleotides, up to about 80 nucleotides, up to about 75 nucleotides, up to about 70 nucleotides, up to about 65 nucleotides, up to about 60 nucleotides, up to about 55 nucleotides, up to about 50 nucleotides, up to about 45 nucleotides, up to about 40 nucleotides) in length.

It is considered that the nucleic acid aptamer can have a length ranging from any of the minimum values described above to any of the maximum values described above. For example, in some aspects, the binging nucleic acid can be from about 40 nucleotides to about 120 nucleotides (e.g., from about 45 nucleotides to about 115 nucleotides, from about 50 nucleotides to about 110 nucleotides, from about 55 nucleotides to about 105 nucleotides, from about 60 nucleotides to about 100 nucleotides, from about 65 nucleotides to about 95 nucleotides, from about 70 nucleotides to about 90 nucleotides, from about 75 nucleotides to about 85 nucleotides, from about 40 nucleotides to about 80 nucleotides, from about 45 nucleotides to about 75 nucleotides, from about 50 nucleotides to about 70 nucleotides, from about 55 nucleotides to about 65 nucleotides, from about 80 nucleotides to about 120 nucleotides, from about 85 nucleotides to about 115 nucleotides, from about 90 nucleotides to about 110 nucleotides, from about 95 nucleotides to about 105 nucleotides) in length.

In some embodiments, the low complexity analyte is a phenethylamine-class compound having an aromatic ring and a primary or secondary amine group. Examples include, but are not limited to, amphetamine, methamphetamine, MDMA, dopamine, norepinephrine, epinephrine, pseudoephedrine, bupropion, and 4-hydroxymethamphetamine. In certain embodiments, the analyte is (+)-methamphetamine.

In some embodiments, the aptamer exhibits less than 25%, 20%, 15%, 10%, 5%, or even 1% cross-reactivity with structural analogs or enantiomers. Cross-reactivity may be expressed as the percentage ratio of the signal produced by an analog to the signal produced by the target at the same molar concentration. Structural analogs may include, but are not limited to, amphetamine, MDMA, dopamine, norepinephrine, epinephrine, pseudoephedrine, bupropion, and 4-hydroxymethamphetamine.

In some aspects, the nucleic acid aptamer can have a dissociation constant (K_{D}) of the phenethylamine-class compound of about 1 pM or more (e.g., about 5 pM or more, about 10 pM or more, about 25 pM or more, about 50 pM or more, about 100 pM or more, about 200 pM or more, about 300 pM or more, about 400 pM or more, about 500 pM or more, about 750 pM or more, about 1 nM or more, about 5 nM or more, about 10 nM or more, about 25 nM or more, about 50 nM or more, about 100 nM or more, about 200 nM or more, about 300 nM or more, about 400 nM or more, about 500 nM or more, about 750 nM or more, about 1 mM or more, about 5 mM or more, about 10 mM or more, about 25 mM or more). In some aspects, the nucleic acid aptamer can have a dissociation constant (K_{D}) of phenethylamine-class compound of about 25 mM or less (e.g., about 10 mM or less, about 5 mM or less, about 1 mM or less, about 750 nM or less, about 500 nM or less, about 400 nM or less, about 300 nM or less, about 200 nM or less, about 100 nM or less, about 50 nM or less, about 25 nM or less, about 10 nM or less, about 5 nM or less, about 1 nM or less, about 750 pM or less, about 500 pM or less, about 400 pM or less, about 300 pM or less, about 200 pM or less, about 100 pM or less, about 50 pM or less, about 25 pM or less, about 10 pM or less, about 5 pM or less, about 1 pM or less). The nucleic acid aptamer can have a dissociation constant (K_{D}) of phenethylamine-class compound ranging from any of the minimum values described above to any of the maximum values described above. For example, in some aspects, the nucleic acid aptamer can have a dissociation constant (K_{D}) to phenethylamine-class compound of from about 1 pM to about 25 mM (e.g., from about 5 pM to about 10 mM, from about 10 pM to about 5 mM, from about 25 pM to about 1 mM, from about 50 pM to about 750 nM, from about 100 pM to about 500 nM, from about 200 pM to about 400 nM, from about 300 pM to about 300 nM, from about 400 pM to about 200 nM, from about 500 pM to about 100 nM, from about 750 pM to about 50 nM, from about 1 nM to about 25 nM, from about 5 nM to about 10 nM, from about 1 pM to about 10 nM, from about 5 pM to about 5 nM, from about 10 pM to about 1 nM, from about 25 pM to about 750 pM, from about 50 pM to about 500 pM, from about 100 pM to about 400 pM, from about 200 pM to about 300 pM, from about 5 nM to about 25 mM, from about 10 nM to about 10 mM, from about 25 nM to about 5 mM, from about 50 nM to about 1 mM, from about 100 nM to about 750 nM, from about 200 nM to about 500 nM, from about 300 nM to about 400 nM).

In some aspects, when phenethylamine-class compound is bound to the nucleic acid aptamer complexed with a detectable signal, one or more properties in the nucleic acid aptamer may change. In some such aspects, the one or more properties of the nucleic acid aptamer can include a conformational change, a difference in melting temperature, and/or a variation in sensitivity to pH or another environmental condition. In some aspects, the changes to one or more properties of the nucleic acid aptamer can cause the detection signal to dissociate from the nucleic acid aptamer.

In some aspects, the detectable output signal can include a fluorophore or a fluorescent dye. In some such aspects, the fluorophore or fluorescent dye can be, but is not limited to, Hydroxycoumarin, Alexa fluor, Aminocoumarin, Methoxycoumarin, Cascade Blue, Pacific Blue, Pacific Orange, Lucifer yellow, Alexa fluor 430, NBD, R-Phycoerythrin (PE), PE-Cy5 conjugates, PE-Cy7 conjugates, Red 613, PeiCP, Cy2, TruRed, FluorX, Fluorescein, FAM, BODIPY-FL, TET, Alexa fluor 532, HEX, TRITC, Cy3, TMR, Alexa fluor 546, Alexa fluor 555, Tamara, X-Rhodamine, Lissamine Rhodamine B, ROX, Alexa fluor 568, Cy3.5 581, Texas Red, Alexa fluor 594, Alexa fluor 633, LC red 640, Allophycocyanin (APC), Alexa fluor 633, APC-Cy7 conjugates, Cy5, Cy5.5, LC red 705, Cy7, IRDye 800 CW, IRDye 700, Cy7.5, Dy780, Dy781, DyLight 800, IRDye 800 CW, Alexa Fluor 647, Alexa Fluor 488, Alexa Fluor 660, Alexa Fluor 680, Alexa Fluor 750, Alexa Fluor 790, JOE, or MAX. As used herein, a dye refers to a chromogenic, fluorogenic, or aggregation-sensitive molecule that produces a detectable signal upon binding, displacement, or conformational change of the aptamer. Dyes suitable for use in the invention include, but are not limited to, thiazole orange, SYBR™ Green, SYBR™ Gold, crystal violet, Nile blue, and malachite green. Aggregation-sensitive dyes may include cyanine-based dyes (e.g., Cy3, Cy5, Cy7 derivatives), indocarbocyanines, or other π-conjugated molecules whose absorbance or fluorescence spectrum changes upon aggregation state alteration. In some embodiments, enzyme-dye conjugates such as horseradish peroxidase with tetramethylbenzidine (TMB), alkaline phosphatase with p-nitrophenyl phosphate (pNPP), or β-galactosidase with chlorophenol red-β-D-galactopyranoside (CPRG) may be used to generate optical signals. In certain embodiments, solvatochromic dyes such as Nile red or Laurdan can be employed to report on polarity changes upon aptamer conformational shifts. In some embodiments, a binding complex modulator refers to any molecular feature or structural component that affects the accessibility of a dye to the aptamer binding region or changes dye aggregation upon analyte binding. Modulators may include secondary structures within the aptamer, such as stem-loops, G-quadruplexes, pseudoknots, or bulged regions, which can act as switchable domains. In some embodiments, aptamers may be engineered to include toehold regions or complementary strands that partially hybridize to the binding sequence and are displaced upon analyte binding.

As used herein, the term aggregation-sensitive dye refers to a chromogenic, fluorogenic, or solvatochromic molecule whose optical properties change measurably when the dye molecules undergo aggregation or disaggregation. Such changes may include shifts in absorbance maxima, alterations in fluorescence emission intensity, wavelength shifts, or variations in scattering. The aggregation state of the dye can be influenced by interactions with nucleic acids, target analytes, or other components in the assay environment. Examples include cyanine dyes (Cy3, Cy5), thiazole orange, SYBR™ Green, and proprietary dyes such as X-732-91B.

As used herein, aggregated dye absorbance refers to the light absorption of dye molecules in an aggregated state, measured primarily within the wavelength range of 400-505 nm. In some embodiments, dye aggregation occurs when the analyte induces a conformational change in an aptamer or other assay components, resulting in a spectral shift toward shorter wavelengths. Such aggregation can serve as a measurable indicator of analyte binding or assay activation.

As used herein, monomeric dye absorbance refers to the light absorption of dye molecules in their non-aggregated, monomeric form, measured predominantly within the wavelength range of 505-620 nm. In some embodiments, in the absence of analyte-induced aggregation, the dye remains primarily in this monomeric state, exhibiting a distinct absorbance profile in the higher wavelength region.

As used herein, fold change refers to the ratio of aggregated dye absorbance to monomeric dye absorbance, calculated to assess the extent of dye aggregation under defined assay conditions. In some embodiments, calculating fold change comprises measuring the absorbance spectrum of the assay solution across the range of 400-620 nm, integrating or averaging the absorbance values from 400-505 nm to obtain the aggregated dye absorbance, integrating or averaging the absorbance values from 505-620 nm to obtain the monomeric dye absorbance, and dividing the aggregated dye absorbance by the monomeric dye absorbance.

In some embodiments, a higher fold change ratio indicates a greater degree of dye aggregation, which may correspond to stronger analyte binding or enhanced assay activation. Conversely, a lower fold change ratio indicates reduced aggregation, which may correspond to weaker analyte binding or minimal assay activation.

In some embodiments, the detectable output signal is optical and is visible to the naked eye within 10 minutes, 5 minutes, or even 1 minute of analyte binding. “Visible to the naked eye” refers to a detectable change in color, brightness, or hue discernible without magnification or specialized equipment under typical lighting conditions.

In certain embodiments, aptamer-target recognition is transduced to an electrochemical signal using redox-tag voltammetry (cyclic, differential pulse, or square-wave voltammetry with tags such as methylene blue, ferrocene, or anthraquinone), electrochemical impedance spectroscopy, amperometric enzyme readouts (e.g., HRP, ALP, GOx couples), potentiometric/open-circuit potential shifts, conductometric or capacitive readouts (including redox-capacitance), field-effect transistor signals (graphene, carbon nanotube, or silicon nanowire devices), chronocoulometry (e.g., RuHex association), electrochemiluminescence, photoelectrochemical photocurrent, anodic or cathodic stripping voltammetry using metal nanoparticle labels, mediator access or blocking with solution mediators (e.g., ferricyanide or RuHex), aptamer-gated nanopore or nanopipette ionic current, G-quadruplex/hemin DNAzyme catalytic currents, direct nucleobase oxidation signals (e.g., guanine peaks), redox cycling at interdigitated microelectrodes, and magnetic bead-electrode enrichment with redox labels; any of the foregoing may be implemented in signal-on or signal-off formats.

### Methods of Detection of a Molecule

In one aspect, disclosed herein is a method for detecting presence of a phenethylamine-class compound in a sample, the method comprising: (a) providing the aptamer in the presence of the phenethylamine-class compound; and (b) measuring output signal, thereby detecting the presence of the phenethylamine-class compound. In specific embodiments, the phenethylamine-class compound can comprise methamphetamine.

In some embodiments, the sample is a seized material or a biological fluid, where the biological fluid includes saliva, blood, serum, plasma, or urine. As used herein, the term biological fluid refers to any liquid derived from a living organism that may contain the analyte of interest. As used herein, a “seized material” refers to any physical sample obtained from a law enforcement, customs, security, or investigative setting that is suspected of containing an illicit, controlled, or otherwise regulated substance. Seized materials may include, but are not limited to, powders, crystals, tablets, capsules, plant material, liquids, or residues collected from surfaces, containers, or packaging. A biological fluid refers to any fluid sample derived from a living organism that is capable of containing the analyte of interest. In certain embodiments, the biological fluid comprises saliva, blood, serum, plasma, or urine. Such fluids may be collected from a human or animal subject, may be used directly or after dilution, filtration, or other sample preparation, and may optionally contain endogenous or exogenous interferents.

In some embodiments, detection in saliva is performed by a dye displacement assay in which analyte binding to the aptamer produces a measurable colorimetric change, the assay operating in saliva or saliva simulant at pH of about 7.4 and ionic strength between about 120 and 160 millimolar, and maintaining at least the stated fold discrimination relative to named structural analogs in the presence of endogenous interferents.

In some embodiments, the sample is saliva or a saliva simulant containing naturally occurring interferents, such as proteins, salts, and mucins, which may challenge assay specificity.

In some embodiments, the nucleic acid aptamer is integrated into a structural context (such as a stem-loop, split aptamer, or conformational switch) that modulates dye accessibility or aggregation state upon analyte binding.

In some embodiments, the nucleic acid aptamer comprises a randomized binding domain of at least 40 nucleotides in length. As used herein, a “randomized binding domain” refers to a contiguous sequence region within the aptamer in which the nucleotide composition is not predetermined, thereby permitting the formation of diverse secondary and tertiary structures during selection. This region may be synthetically generated to include a substantially uniform distribution of the four standard nucleotides (adenine, cytosine, guanine, and thymine for DNA aptamers; adenine, cytosine, guanine, and uracil for RNA aptamers) or may be biased toward specific bases to influence structural properties or target affinity.

In certain embodiments, the randomized domain is enriched in thymine residues, such that thymine accounts for at least 40% of the nucleotides within the domain. Thymine enrichment can be advantageous for modulating aptamer folding, increasing flexibility, and enhancing hydrophobic or π-stacking interactions with low complexity analytes such as phenethylamine-class compounds. In some embodiments, the thymine content may be at least 45%, at least 50%, at least 55%, or at least 60%, depending on the structural and functional requirements of the aptamer.

In some embodiments, the aptamer sequence exhibits (e.g., at least about 80%, at least about 82%, at least about 85%, at least about 88%, at least about 90%, at least about 92%, at least about 95%, or at least about 98% sequence identity to any one of SEQ ID NOS: 45, 46, 57, 64, 65, and 66, as determined by standard sequence alignment algorithms. Examples of the sequence alignment algorithms include BLAST, ClustalW, Needleman-Wunsch, Smith-Waterman, MUSCLE, and MAFFT. Sequence identity may be calculated by aligning the candidate sequence to a reference sequence using one of these algorithms with default or specified parameters, determining the number of identical residues in the alignment, dividing by the alignment length, and multiplying by 100. For instance, BLASTN with default settings can be used to determine that an aptamer sequence exhibits at least 90% identity to a reference sequence.

### Method of Isolating an Aptamer

In one aspect, disclosed herein is a method of identifying an aptamer specific for a phenethylamine-class compound, wherein the aptamer exhibits less than 25% cross-reactivity to a structural analog or an enantiomer of the phenethylamine-class compound. The method comprises providing a nucleic acid library comprising a randomized sequence region greater than 40 nucleotides in length, hybridizing the nucleic acid library to a complementary capture strand, immobilizing the hybridized library on a solid support that binds the affinity tag, contacting the immobilized library with a sample containing the phenethylamine-class compound in a buffer, performing counter-selection by contacting the library with one or more non-target molecules including at least one structural analog or enantiomer of the phenethylamine-class compound, and identifying nucleic acids that are specific for the phenethylamine-class compound. Also disclosed herein, the phenethylamine-class compound is methamphetamine, the complementary capture strand comprises an affinity tag, the affinity tag is biotin, and the solid support is a streptavidin-coated agarose bead support. In certain embodiments, the non-target molecules are selected from the group comprising amphetamine, bupropion, 3,4-methylenedioxymethamphetamine, 4-hydroxymethamphetamine, norepinephrine, epinephrine, dopamine, tyramine, pseudoephedrine, quinine, diphenhydramine, procaine, lidocaine, methylphenidate, and alprazolam. As used herein, a “solid support,” refers to any material capable of immobilizing nucleic acids via covalent or non-covalent interactions. Solid supports include, but are not limited to, agarose beads, magnetic beads, glass slides, polystyrene microtiter plates, and nitrocellulose membranes. Immobilization agents may include biotin-streptavidin binding pairs, His-tag nickel chelate interactions, or covalent crosslinkers such as N-hydroxysuccinimide (NHS) esters and maleimides.

Also disclosed herein are methods of isolating an aptamer that specifically binds a target compound comprises: providing a nucleic acid library comprising a randomized sequence region; hybridizing the nucleic acid library to a complementary capture strand comprising an affinity tag; immobilizing the hybridized library on a solid support that binds the affinity tag; contacting the immobilized library with a sample containing the target compound in a buffer; performing counter-selection by contacting the library with one or more non-target molecules; washing the immobilized library to remove unbound sequences; eluting sequences that remain bound to the target compound; amplifying the eluted sequences; and repeating for multiple selection rounds.

In some embodiments, the affinity tag is biotin. In some embodiments, the solid support that binds the affinity tag is a streptavidin-coated agarose bead support. In some embodiments, the randomized sequence region comprises at least 40 nucleotides. In some embodiments, the target compound is (+)-methamphetamine. In some embodiments, the buffer contains magnesium ions (Mg²⁺) and/or calcium ions (Ca²⁺). In some embodiments, the non-target molecules are selected from amphetamine, bupropion, 3,4-methylenedioxymethamphetamine, 4-hydroxymethamphetamine, norepinephrine, epinephrine, dopamine, tyramine, pseudoephedrine, quinine, diphenhydramine, procaine, lidocaine, methylphenidate, and alprazolam. In some embodiments, the eluted sequences are amplified using polymerase chain reaction (PCR). In some embodiments, progress between selection rounds is monitored by measuring the amount of nucleic acid sequences released in the presence of the target compound compared to a no-target control. In some embodiments, the aptamers bound to the target compound comprise a stem loop architecture. In some embodiments, the aptamers bound to the target compound exhibit less than 25% cross-reactivity to the non-target molecules.

### Screening Assays and Kits

In one aspect, disclosed herein is a screening assay for determining the presence of a phenethylamine-class compound in a sample, wherein the screening assay comprises an aptamer comprising a nucleic acid that specifically binds to the phenethylamine-class compound, wherein the aptamer exhibits less than 25% cross-reactivity to a structural analog or an enantiomer of the phenethylamine-class compound, and a detectable output signal that is detectable upon binding of the nucleic acid to the phenethylamine-class compound. In some embodiments, the phenethylamine-class compound is methamphetamine. In some embodiments, the aptamer comprises a nucleic acid sequence as set forth in any one of SEQ ID NOS: 45, 46, 57, 64, 65, and 66, optionally comprising at least one additional mutation and/or at least one additional non-natural nucleic acid, wherein the mutation may comprise a thymine at a position corresponding to T18 or T39 that decreases affinity by at least 20-fold. In certain embodiments, the nucleic acid comprises a randomized region of at least 40 nucleotides that is thymine rich with at least 40 percent thymine content. In some embodiments, the detectable signal is optically visible and comprises a colorimetric dye detectable upon displacement.

In one aspect, disclosed herein is an aptamer-based colorimetric assay for detecting (+)-methamphetamine in a sample comprises contacting the sample with a nucleic acid aptamer that binds to (+)-methamphetamine, wherein the aptamer is complexed with an aggregation-sensitive dye, and detecting an optical change resulting from displacement of the dye or alteration of its aggregation state upon binding of the (+)-methamphetamine.

In one aspect, disclosed herein is a kit for detecting a low-epitope small-molecule analyte in a sample. The kit comprises a nucleic acid aptamer that specifically binds the analyte, the aptamer being linked to, or in functional association with, a detectable signal, such as a colorimetric reporter system configured to produce a measurable optical change upon analyte binding, or an electrochemical signal. An optical change may be a visible color shift, a change in absorbance intensity, or both, detectable by the unaided eye or by an optical instrument. The kit further includes one or more reagents for performing the assay under predetermined conditions of pH and ionic strength, which may be selected to optimize aptamer-analyte binding and minimize nonspecific interactions. As used herein, predetermined conditions refer to buffer formulations, ionic compositions, and temperature ranges pre-selected for optimal assay performance. The kit additionally contains printed or electronic instructions detailing preparation of reagents, assay execution, and interpretation of results, including reference controls for distinguishing positive from negative detections.

In one aspect, disclosed herein is a kit for detecting a low-epitope small-molecule analyte in a sample. The kit comprises a nucleic acid aptamer that specifically binds the analyte, the aptamer being linked to, or in functional association with, a detectable signal, such as a colorimetric reporter system configured to produce a measurable optical change upon analyte binding, or an electrochemical signal.

In another aspect, disclosed herein is a system for detecting a low-epitope small-molecule analyte in a sample. The system comprises a colorimetric assay module containing a nucleic acid aptamer that specifically binds the analyte, the aptamer being linked to, or in functional association with, a detectable signal, such as a colorimetric reporter system configured to produce a measurable optical change upon analyte binding, or an electrochemical signal.

In some embodiments, the detection unit comprises a portable spectrophotometer, a smartphone-based optical sensor, or a handheld device with integrated light source and detector. In some embodiments, the system is configured to apply predetermined specificity thresholds relative to two or more structural analogs of the analyte, generating a positive detection only when the observed signal surpasses those thresholds.

### EXAMPLES

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention that are apparent to one skilled in the art.

### Example 1: Examining the Relationship Between Aptamer Complexity and Molecular Discrimination of a Low-Epitope Target

Aptamers are oligonucleotides that recognize specific molecules with high affinity. They have several favorable attributes relative to conventional protein-based receptors like antibodies, such as low cost, ease of synthesis, straightforward sequence engineering, high stability, reversible denaturation, and the capability to tune their binding properties. Aptamers are increasingly being utilized as bioreceptors in sensors for medical diagnostics, health monitoring, food safety, and forensics. Given their simplicity and ease of use, aptamer-based assays have unique advantages over methods such as mass spectrometry in terms of turnaround time and cost. Moreover, their distinctive advantages are enabling unprecedented biosensing applications that are beyond the reach of antibodies, such as real-time molecular detection in live cells, tissues, organs, and blood circulation. Aptamers are isolated from randomized libraries through an in vitro method known as systematic evolution of ligands by exponential enrichment (SELEX). Here, the library is incubated with the target, after which aptamers are separated from binding-incompetent sequences and amplified. The enriched pool of sequences resulting from this process is subjected to another cycle of selection until the pool exhibits satisfactory binding properties.

While there have been numerous successes in the generation of aptamers for protein targets, the isolation of aptamers for small molecules remains challenging. Historically, these selections have been impeded by the need to covalently attach the small-molecule target to a solid support to facilitate isolation of target-binding sequences. Such conjugation is chemically challenging, especially for targets that lack functional groups amenable to covalent linkage, and also masks the very few structural elements that aptamers could bind to, resulting in aptamers with low affinity and specificity. Nutiu and Li effectively addressed these limitations by immobilizing the library rather than the target onto microbeads that have been coupled to complementary DNA (cDNA) strands that hybridize to a portion of the library sequence (Nutiu R.; Li Y. In Vitro Selection of Structure-Switching Signaling Aptamers. Angew. Chem., Int. Ed. 2005, 44 (7), 1061-1065). When challenged with target, the target binders dissociate from the cDNA, facilitating their separation from the beads and binding-incompetent sequences. With further refinements and simplification by the Stojanovic group, (Yang K.-A.; Pei R.; Stojanovic M. N. In Vitro Selection and Amplification Protocols for Isolation of Aptameric Sensors for Small Molecules. Methods 2016, 106, 58-65) this library-immobilized SELEX method now represents the most reliable means of identifying aptamers for small-molecule targets. For instance, many have utilized this selection strategy to isolate high-affinity DNA aptamers with good specificity for tetrahydrocannabinol (Yu et al. Isolation of Natural DNA Aptamers for Challenging Small-Molecule Targets, Cannabinoids. Anal. Chem. 2021, 93 (6), 3172-3180) and theophylline (Huang et al. A DNA Aptamer for Theophylline with Ultrahigh Selectivity Reminiscent of the Classic RNA Aptamer. ACS Chem. Biol. 2022, 17 (8), 2121-2129) and RNA aptamers for paromomycin (Boussebayle et al. Next-Level Riboswitch Development-Implementation of Capture-SELEX Facilitates Identification of a New Synthetic Riboswitch. Nucleic Acids Res. 2019, 47 (9), 4883-4895) as well as guanine and quinine (Mohsen et al. Exploiting Natural Riboswitches for Aptamer Engineering and Validation. Nucleic Acids Res. 2023, 51(2), 966-981) Nevertheless, the selection of aptamers for targets that have very few distinct epitopes still requires careful experimental design, trial and error, and optimization. With only two successes reported thus far in the form of DNA aptamers (Nakatsuka et al. Aptamer-Field-Effect Transistors Overcome Debye Length Limitations for Small-Molecule Sensing. Science 2018, 362 (6412), 319-324; Huang et. al. Simultaneous Detection of L-Lactate and D-Glucose Using DNA Aptamers in Human Blood Serum. Angew. Chem., Int. Ed. 2023, 62 (12)), there is still a lack of insight into how to reliably isolate high-quality aptamers for such challenging targets. For example, while isolating aptamers for 7-amino butyric acid, a molecule with just an amino and a carboxyl moiety, the Stojanovic group encountered multiple failures using an N36 library and only finally achieved success with an N44 library (Yang et al. A Functional Group-Guided Approach to Aptamers for Small Molecules. Science 2023, 380 (6648), 942-948). A detailed account and examination of such selection trials and parameters (e.g., length of the random region) is valuable in terms of enabling the design of successful selection experiments for other problematic targets.

To address this knowledge gap and examine how selection conditions influence the aptamer isolation process, a series of independent SELEX experiments were performed to isolate aptamers for the small-molecule drug (+)-methamphetamine. The reasoning for choosing this target was 3-fold. First, aptamers that bind (+)-methamphetamine would be of high value given the prevalence of (+)-methamphetamine abuse and its impact on public health, and hence the need for sensors that can detect this drug in seized substances and biological samples. Second, (+)-methamphetamine is a very low-complexity target and thus a formidable challenge for selecting aptamers with essentially two functional groups available for recognition by oligonucleotides: a phenyl group and an amine. Finally, there are at least three different published studies from the past decade describing efforts to isolate aptamers that bind to methamphetamine, (Ebrahimi et al. 2013; Bor et. al. Synthetic Antibodies for Methamphetamine Analysis: Design of High Affinity Aptamers and Their Use in Electrochemical Biosensors. J. Electroanal. Chem. 2022, 921, 116686; Sester et al. 2022) providing the opportunity to systematically study the impact of different selection strategies and conditions on aptamer quality. First it was determined that previously reported methamphetamine aptamers either have weak or no affinity for this target. Then selection experiments four trials in total were performed. Throughout these trials, it was determined that while it is possible for aptamers to bind (+)-methamphetamine, the capability to discriminate this target from structurally similar molecules necessitated more complex aptamers with larger binding domains. Eventually, new aptamers with excellent specificity for (+)-methamphetamine, with 50-fold and 89-fold greater affinity for this target relative to amphetamine and MDMA, respectively, were identified, surpassing the capabilities of existing antibodies. The results provide valuable insights into how best to execute selections for low-complexity targets in the future.

### Materials and Methods

Phosphate buffered saline (10×, molecular biology grade) and molecular biology grade water were purchased from Corning. Magnesium chloride solution for molecular biology was purchased from Sigma-Aldrich. Procaine hydrochloride, 1-tyrosine, 1-phenylalanine, quinine hemisulfate salt monohydrate, caffeine, diphenhydramine HCl, cocaine HCl, sodium dodecyl sulfate, serotonin HCl, tyramine, 3,4-dihydroxyphenylacetic acid and dopamine HCl were purchased from Sigma-Aldrich. Lidocaine hydrochloride monohydrate was purchased from Alfa Aesar. Morphine sulfate hydrate, fentanyl HCl, alprazolam, (+)-methamphetamine HCl, (−)-methamphetamine HCl, amphetamine HCl, bupropion HCl, 3,4-methylenedioxypyrovalerone (MDPV) HCl, 3,4-methylenedioxymethamphetamine (MDMA) HCl, methylphenidate HCl, methadone HCl, 4-hydroxymethamphetamine HCl, 4-hydroxyamphetamine HCl, homovanillic acid, (+)-pseudoephedrine HCl, epinephrine HCl, and norepinephrine bitartrate hydrate were purchased from Cayman Chemicals. SYBR™ Gold and streptavidin-coated agarose resin were purchased from Thermo Fisher Scientific. X-732-91B dye was retrieved from the Max Weaver Dye Library at North Carolina State University. Microgravity columns (500 μL) were purchased from Bio-Rad. GoTaq Hot Start Colorless Master Mix was purchased from Promega. AMICON™ Ultra centrifugal filters (3 kDa MWCO) were purchased from Sigma-Aldrich. The QIAQUICK™ PCR purification kit was purchased from Qiagen. EDTA (0.5 M, pH 8.0) and formamide were purchased from Fisher Scientific. Exonuclease I (Exo I, E. coli, 20 U/μL) and Exonuclease III (Exo III, E. coli, 100 U/μL) and T5 exonuclease (T5 Exo, 10 U/μL) were purchased from New England Biolabs. All other chemicals were purchased from Sigma-Aldrich unless otherwise specified. Ultrapure water (resistivity=18.2 MΩ·cm, 25° C.) was obtained from a Milli-Q EQ 7000 water purification system.

### Oligonucleotides

All DNA oligonucleotides for SELEX (see Table 1 for sequences) were purchased from Integrated DNA Technologies. The random library and sequencing primers were PAGE purified, and SELEX PCR primers and complementary DNA (cDNA15-bio) were HPLC purified. All other DNA oligonucleotides were purified by standard desalting. DNA was dissolved in molecular biology grade water, and concentrations were determined using a NANODROP™ 2000 Spectrophotometer.

### SELEX Procedure

Four different trials of library-immobilized SELEX were performed to isolate DNA aptamers that bind to (+)-methamphetamine. The basic procedure follows a previously reported protocol, (Yang et al. 2016) and details of the selection process are provided in Tables 2-5. Briefly, the random or enriched library was mixed with a 15-nt biotinylated complementary DNA (cDNA15-bio) (SEQ ID NO:6 or SEQ ID NO: 11) at a molar ratio of 1:5 in 250 μL selection buffer, consisting of 1× PBS diluted from 10× PBS (101.4 mM Na₂HPO4, 17.6 mM KH₂PO₄, 1369 mM NaCl, 27 mM KCl) and 1 mM MgCl₂, heated at 95° C. for 10 min, then slowly cooled to room temperature over 20 min to allow the library to hybridize with the cDNA. Meanwhile, a 500 μL microgravity column was filled with 300 μL of molecular biology grade water and subjected to vacuum degassing for 1 min. 250 μL of streptavidin-coated agarose resin was then loaded into the column and washed five times with 250 μL selection buffer. The hybridized library-cDNA complexes were added to the column for immobilization onto the agarose resin. The eluate was collected and flowed through the column three times to maximize library loading onto the agarose resin. The column was then washed with 250 μL selection buffer 10-50 times to remove sequences that weakly bind to the cDNA. Afterward, 250 μL of (+)-methamphetamine dissolved in selection buffer was added to the column, displacing (+)-methamphetamine-binding sequences from the biotinylated cDNA into the eluate. This step was performed three times, and the eluate was combined and then purified using a 3 kDa MWCO filter to remove (+)-methamphetamine and salts and concentrate the solution to <100 μL. The enriched library was then PCR amplified using 600 μL GoTaq Hot Start Colorless Master Mix (2×) with 1 μM forward primer (FP) and 1 μM biotinylated reverse primer (RP-bio). PCR was performed using a Bio-Rad C1000 thermal cycler with the following conditions: 2 min at 95° C.; 9-13 cycles of 95° C. for 15 s, 58° C. for 30 s, and 72° C. for 45 s; 5 min at 72° C. The optimal number of amplification cycles was confirmed by 15% PAGE. Single-stranded DNA was prepared as reported previously, (Yang et al. 2016) and then purified and concentrated by a 3 kDa MWCO filter. The concentration was determined by a NanoDrop 2000 spectrophotometer. Counter-SELEX was performed from round 2 in trials 1, 3, and 4 to remove sequences that bound to interferent molecules. After the beginning of selection buffer wash, 250 μL of various counter-targets dissolved in selection buffer were added to the column. Detailed information regarding counter-targets is provided in Tables 2-5. After counter-SELEX, the column was washed with selection buffer 10-40 times to remove any residual counter-targets and nonspecific binders. Positive selection with (+)-methamphetamine was then performed.

### High-Throughput Sequencing (HTS) of Pools and Bioinformatic Analysis

Selection rounds 13 and 19 from trial 1; rounds 9 and 11 from trial 2; rounds 7, 13, and 18 from trial 3; and rounds 9, 10, 11, 13, 14, and 15 from trial 4 were subjected to Illumina-based HTS by Azenta Life Sciences. To prepare SELEX pools for sequencing, 100 nM of each SELEX pool was subjected to PCR amplification using 1 μM of customized forward and reverse primers containing partial Illumina adapters. The PCR conditions employed were as follows: 2 min at 95° C.; 10 cycles of 95° C. for 15 s, 58° C. for 30 s, and 72° C. for 45 s; and 5 min at 72° C. The PCR products were then cleaned using the QIAquick PCR purification kit, and the successful addition of adapters was confirmed using denaturing polyacrylamide gel electrophoresis (PAGE), after which 25 μL of the 20 ng/μL purified pool was submitted for sequencing. Each pool yielded ˜300,000-1,345,800 reads. Prior to analysis, complementary sequences were converted to their reverse complement using the fastx toolkit and combined with the forward reads. The constant region was then removed using cutadapt, and sequences containing ‘N’ nucleotides in the random region were discarded. Finally, FASTAptamer was used to determine the abundance of each unique sequence as well as its enrichment-fold throughout several SELEX rounds. Summary HTS statistics for trials 1-4 is provided in Table 6. Aptamer families were discovered using the Raptgen software.

### Exonuclease Digestion Fluorescence Assay for Aptamer Specificity Screening

The exonuclease digestion fluorescence assay was performed. Each aptamer (final concentration: 0.5 μM) was diluted in PBS buffer (final concentration: 1×, pH 7.4) and heated to 95° C. for 10 min, then immediately cooled on ice for 1 min. MgCl₂ (final concentration: 1 mM), and BSA (final concentration: 0.1 mg/mL) were added immediately. Five μL of this aptamer solution was added to 20 μL of selection buffer, (+)-methamphetamine dissolved in selection buffer (final concentration: 250 or 500 μM), or interferents (procaine, lidocaine, caffeine, quinine, diphenhydramine, amphetamine, cocaine, homovanillic acid, methylphenidate, (+)-pseudoephedrine, alprazolam, epinephrine, bupropion, methadone, morphine, 3,4-Methylenedioxypyrovalerone (MDPV), 3,4-Methylenedioxymethamphetamine (MDMA), fentanyl, dopamine, 4-hydroxymethamphetamine (4-HMA), 4-hydroxyamphetamine (4-HA), phenylalanine, 3,4-dihydroxyphenylacetic acid (DOPAC), norepinephrine, tyrosine, tyramine, or serotonin) dissolved in selection buffer (final concentration: 250 M, except for alprazolam which was 50 μM and included 5% DMSO in the buffer). The mixture was incubated at 25° C. for 30 min, after which 25 μL of Exo III and Exo I (final concentrations 0.025 U/μL and 0.05 U/μL, respectively) or T5 Exo and Exo I (final concentrations 0.2 U/μL and 0.015 U/μL, respectively) in selection buffer containing 0.1 mg/mL BSA was added to begin the digestion reaction. Five μL of sample was collected at various time-points and added to 30 μL of quenching solution (1× PBS, 1.16× SYBR™ Gold, 25 mM EDTA, 14.6% (v/v) formamide) in the wells of a 384-well black microplate. SYBR™ Gold fluorescence was recorded using a Tecan Spark plate reader (excitation: 495 nm, emission: 537 nm, bandwidth: 5 nm). Fluorescence was plotted against each time-point to construct time-course digestion plots of each sample. Enzymatic inhibition was measured in terms of the resistance value, which is calculated using the formula (AUCₜ/AUC₀)−1, where AUC and AUC₀ are the areas under the curve of the time-course data with and without target, respectively. The integration time was customized for each aptamer and was chosen as the point at which fluorescence reached 10% of its initial value. The fluorescence of each sample was recorded 10 times, and average values were used for analysis.

### Isothermal Titration Calorimetry (ITC)

All experiments to determine binding aptamer affinity using ITC were conducted on a Malvern MICROCAL PEAQ-ITC™, and the data were analyzed with MICROCAL PEAQ-ITC™, Analysis Software using a one-site binding model. Each aptamer was tested at 23° C. with both aptamer and target dissolved in either selection buffer described here or buffers reported previously in the literature. For the determination of methamphetamine affinity, 300 to 5000 μM (+)-methamphetamine HCl was titrated into 20 to 100 μM aptamer during a single titration run. The run consisted of a 60 s equilibration followed by one 0.4-μL injection to purge the syringe, then 19 successive 2 μL injections with either 180 s or 150 s spacing. For amphetamine affinity measurements, 500 or 1,500 μM amphetamine HCl was titrated into 20 or 40 μM aptamer. The runs again consisted of a 60 s equilibration with a single 0.4-μL purge injection and 19 times of 2 μL injections, all with 180 s spacing. For some of the aptamers, a double titration of amphetamine was required to reach saturation. This double titration was performed by first running a single titration as described above, but not emptying the cell upon completion. Instead, only the overflow of the sample cell was removed; the syringe was reloaded with target, and a second titration was started with the same parameters as the first. To combine the two experiments, MICROCAL™ ITC software was used. Specific conditions and experimentally determined binding affinity and thermodynamic parameters are presented in Tables 7-8.

### Optimizing Aptamer-Dye Ratio for Detection of (+)-Methamphetamine Via Dye-Displacement Assay

All aptamer-based dye-displacement assays were conducted at room temperature. 400 μM dye X-732-91B was prepared in DMSO, and 1 μL was pipetted to the bottom of PCR tubes. Then, 99 μL of aptamer ML4 solution, prepared at a final concentration of 0-10 μM in selection buffer containing 0.01% SDS and 0.001% Triton X-100, was added to each PCR tube to form aptamer-dye complexes. After gentle mixing and centrifugation, 75 μL of the aptamer-dye complex was loaded into the wells of a transparent 384-well plate. Absorbance spectra were recorded at 0, 5, and 10 min from 300-900 nm with 5 nm step size using a Tecan Spark microplate reader. All plots were generated in Origin 2023b. Dye monomers and aggregates were respectively calculated as the area under the curve (AUC) from 505-620 nm and 400-505 nm. The samples were then transferred to a white 384-well plate for photography with a Nikon D750 camera.

### (+)-Methamphetamine Detection in Buffer and Saliva via Dye-Displacement Assay

For detection in buffer, aptamer and dye were mixed in the PCR tube at a final concentration of 4 μM dye and 6 μM aptamer. 50 μL of the aptamer-dye complex was immediately added to PCR tubes containing 50 μL of (+)-methamphetamine or interferents in selection buffer. For the (+)-methamphetamine calibration curve, the final concentration ranged from 0-200 μM. For specificity testing, the interferents were present at 50 μM with target at either 25 or 50 μM. For detection in saliva, pooled saliva was collected from four drug-free, healthy, consenting individuals (three male, one female). The saliva was first centrifuged for 30 min at 20,000 rcf to remove any solid matter, and the supernatant was then filtered using a 0.22-μm filter. Drug-spiked saliva was prepared by adding 5 μL of target at various concentrations into 50 μL of 100% saliva. Then, 45 μL of dye-aptamer complex was added to the spiked saliva and mixed for 10 s. Thus, the saliva is diluted by 50% in 1× buffer. The final concentrations of dye-aptamer complexes and target were the same as for the calibration in buffer. 75 μL of the sample mixture was immediately loaded into the wells of a 384-well plate for absorbance measurements in a Tecan Spark microplate reader using the same settings as for the aptamer optimization. Data analysis was conducted in Origin 2023b, using signal gain as the metric for target detection. Signal gain was calculated as (R−R₀)/R₀, where R and R₀ are the ratio of aggregate (400-505 nm) to monomer (505-620 nm) AUC for samples with and without target/interferent. Samples were photographed after transfer to a white 384-well plate with a Nikon D750 camera.

### Results

### Assessment of Previously Reported Methamphetamine Aptamers

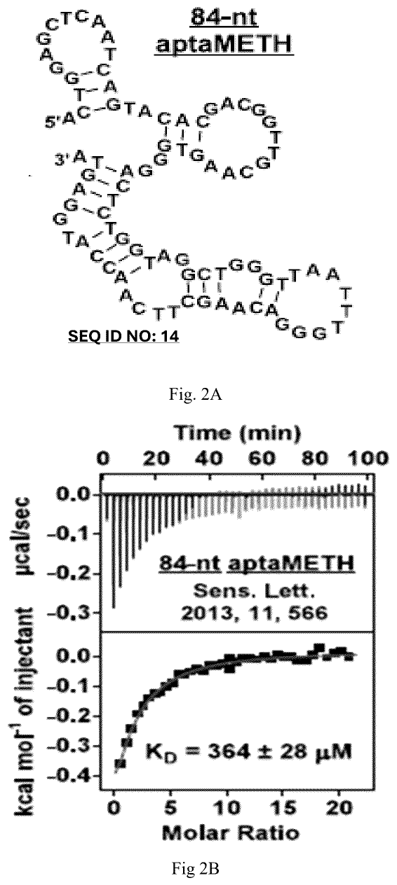

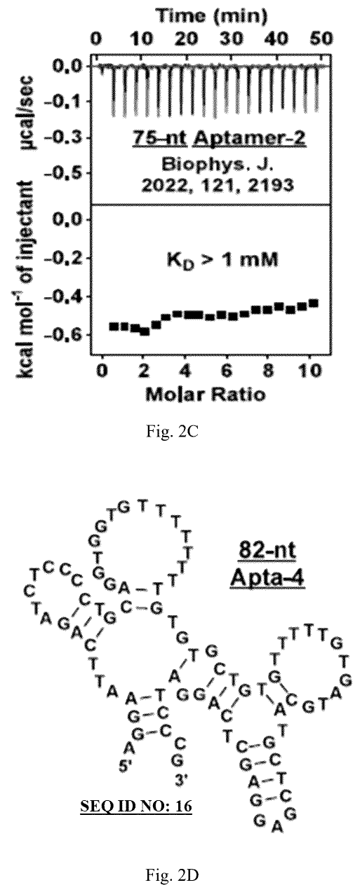

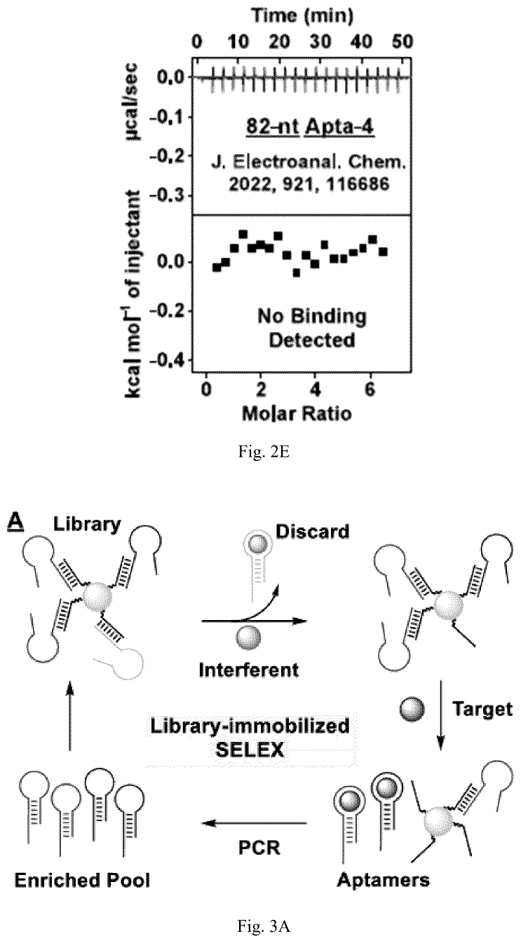

The first SELEX experiment to isolate aptamers for methamphetamine was conducted by Ebrahimi et al. more than a decade ago (Ebrahimi et al. 2023). Ebrahimi et al. covalently attached methamphetamine onto epoxy-modified agarose via its amino group to partition aptamers from binding-incompetent sequences in a randomized N40 DNA library using target-immobilized SELEX. The selection buffer consisted of 20 mM Tris (pH 7.4) with a relatively high ionic strength (200 mM NaCl and 5 mM MgCl₂). After 14 rounds of SELEX, they identified aptaMETH (FIG. 2A), an 84-nt aptamer that reportedly binds methamphetamine with a K_{D} of 100 nM as determined using a bead-based binding assay. To confirm the ability of this aptamer to bind methamphetamine, aptaMETH was synthesized (Table 9) and an exonuclease fluorescence assay was utilized to determine its relative target affinity. In the assay, aptamers are digested by T5 exonuclease (T5 Exo) and exonuclease I (Exo I); ligand-bound aptamers exhibit resistance to digestion that is proportional to their ligand-binding affinity. It was observed that digestion was inhibited in the presence of racemic methamphetamine, indicating that the aptamer indeed binds to this target. However, the degree of enzymatic inhibition was relatively low, with maximal inhibition occurring at an unexpectedly high concentration of 500 μM methamphetamine. To formally quantify the affinity of this aptamer, the gold-standard method isothermal titration calorimetry (ITC) was used. Methamphetamine exists as two enantiomers: (+)- and (−)-methamphetamine, aptamer affinity to each enantiomer was assessed separately. It was confirmed that aptaMETH binds (+)-methamphetamine, but its affinity is more than 3 orders of magnitude weaker (K_{D}=364±28 μM) (FIG. 2B) than the previously reported K_{D} of 100 nM. The affinity of the aptamer for (−)-methamphetamine was too weak to confidently quantify (K_{D}>1 mM), demonstrating that aptamer-target interactions are stereospecific. The low enthalpy of binding indicated by ITC, coupled with the fact that the target was conjugated to a solid support during SELEX, indicates that the aptamer recognizes a part, but not the entirety, of the methamphetamine molecule. It is likely that the aptamer binds well to bead-immobilized methamphetamine, but poorly to methamphetamine free in solution. However, Xie et al. recently utilized a truncated variant of aptaMETH (38-nt aptaMETH (SEQ ID NO: 15); Table 9) to detect methamphetamine in an electrochemical aptamer-based sensor, with a reported limit of detection (LOD) of 30 nM. ITC was performed to determine if this truncated aptamer had affinity for methamphetamine, but no affinity was observed for either (+)- or (−)-methamphetamine. Similarly, Sester et al. performed target-immobilized SELEX to isolate a DNA aptamer for methamphetamine with an N40 random library in 2 mM Tris-HCl buffer with relatively low ionic strength (10 mM NaCl, 0.5 mM KCl, 0.2 mM MgCl₂, and 0.1 mM CaCl₂)). Their highest-affinity aptamer (Aptamer-2 (SEQ ID NO: 16); Table 9) exhibited a K_{D} of 244 nM in a dye-displacement assay. However, in the exonuclease fluorescence assay, Aptamer-2 did not resist digestion even in the presence of 500 μM (+)-methamphetamine, indicating minimal target binding. The ITC data indicated that Aptamer-2 has very weak or no binding affinity for both enantiomers of methamphetamine (K_{D}>1 mM) (FIG. 2C). Thus, it was concluded that target-immobilized SELEX is not a suitable means of isolating high-affinity aptamers for a target like methamphetamine, with so few functional groups.

In another recent report, Bor et al. performed graphene-oxide SELEX to isolate DNA aptamers for methamphetamine from an N40 DNA library. The library was first non-specifically absorbed onto a graphene oxide surface, after which the target was added. Aptamers capable of binding the target should desorb from graphene oxide and can be collected in the supernatant. The PBS selection buffer had a pH of 7.0 with moderate ionic strength (100 mM NaCl, 2.7 mM KCl, and 2 mM MgCl₂). After eight rounds, they identified an 82-nt aptamer termed Apta-4 (FIG. 2D (SEQ ID NO: 17), and Table 9) and determined that it binds methamphetamine with a K_{D} of 1.3 μM based on ITC analysis. However, the exonuclease assay revealed that this aptamer did not resist enzymatic digestion, even in the presence of 500 μM racemic methamphetamine again, indicating weak or no affinity. Likewise, when ITC was performed under the same conditions as reported by Bor et al., no affinity between Apta-4 and (+)-methamphetamine (FIG. 2E) or (−)-methamphetamine was observed. Even when ITC was performed with a 3-fold higher aptamer and target concentration, no affinity was observed. These data indicate that Apta-4 does not bind to methamphetamine, and that the graphene-oxide SELEX effort had failed.

### First Trial of SELEX: An Alternative Partitioning Strategy-Library-Immobilized SELEX

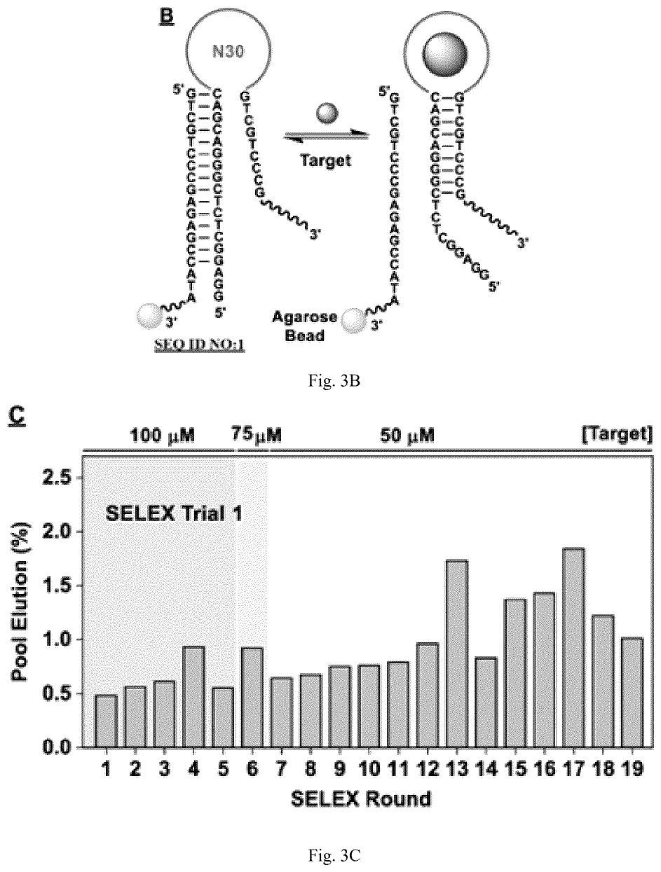

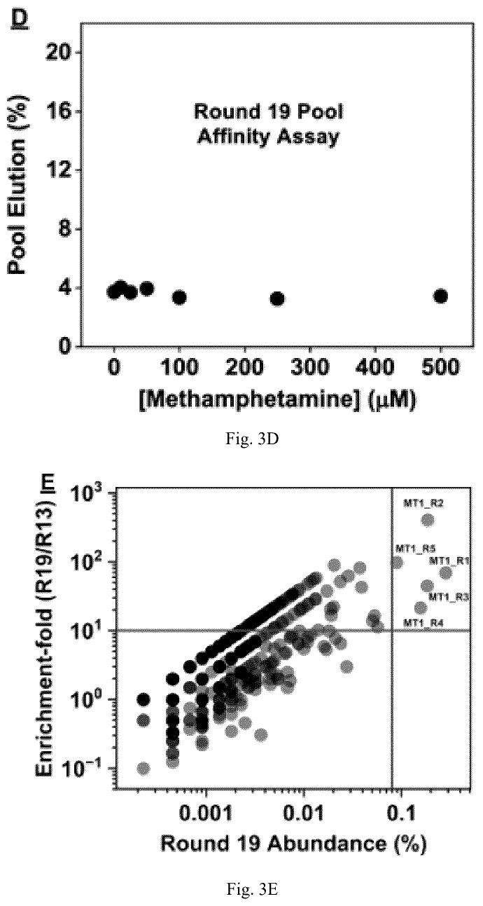

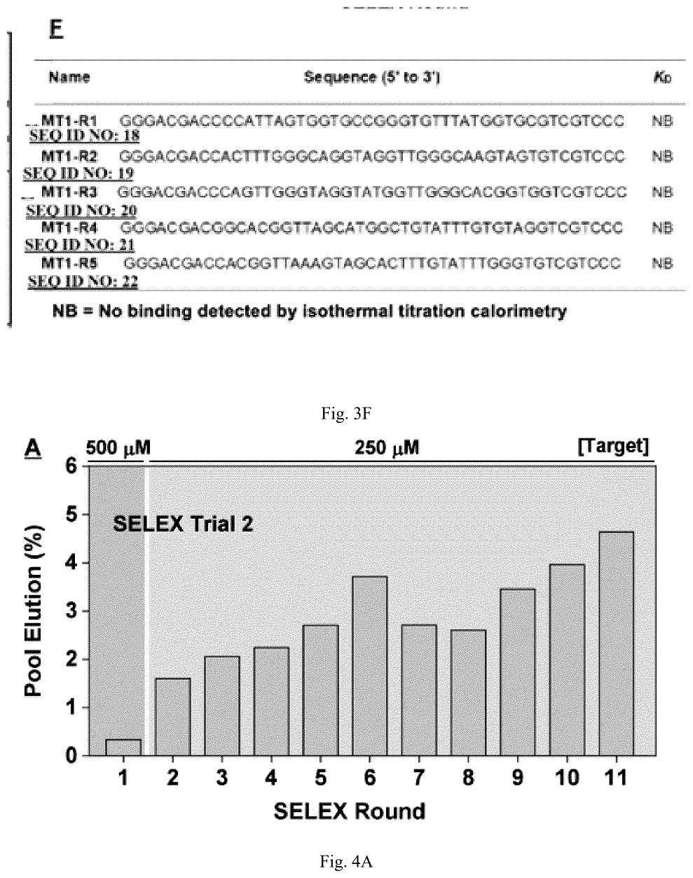

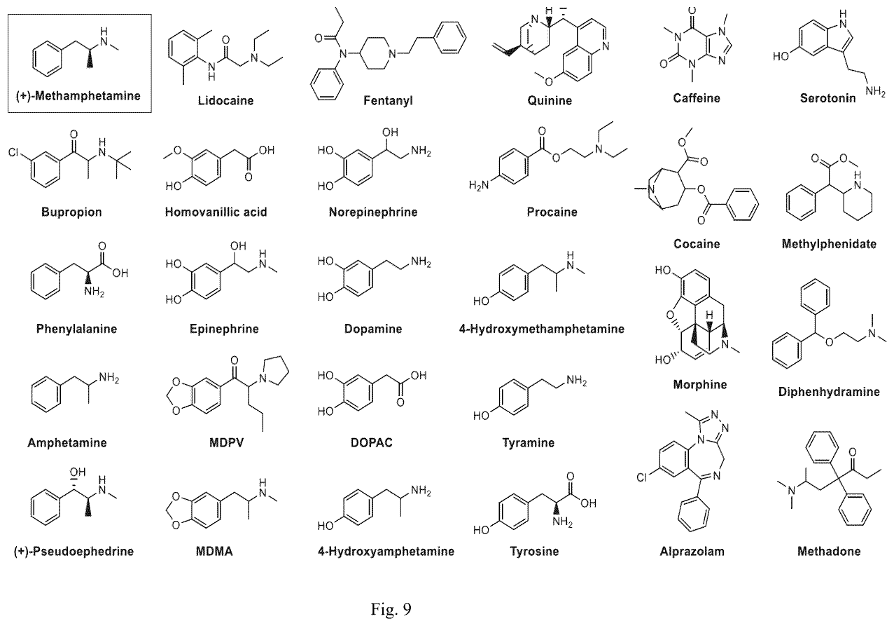

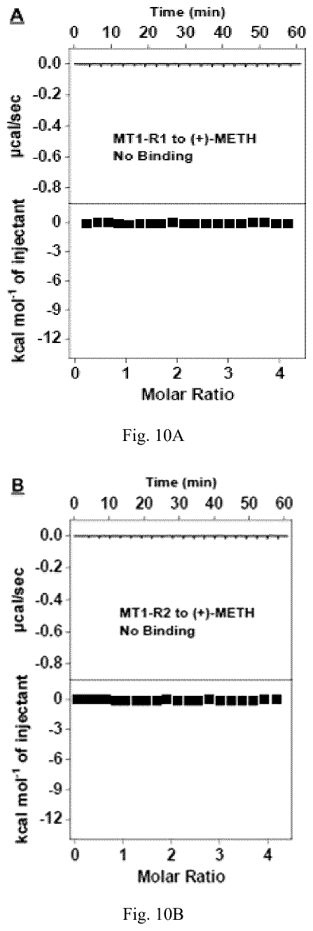

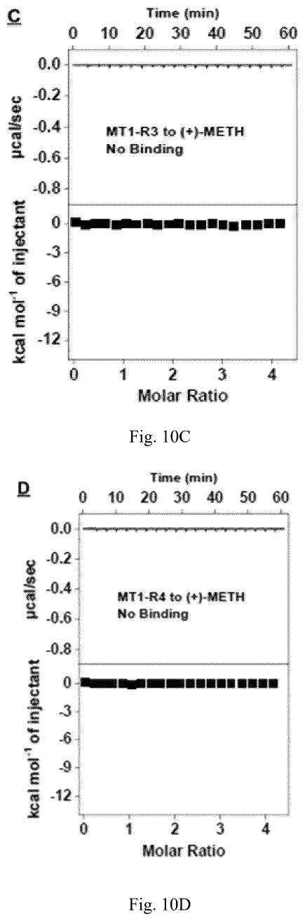

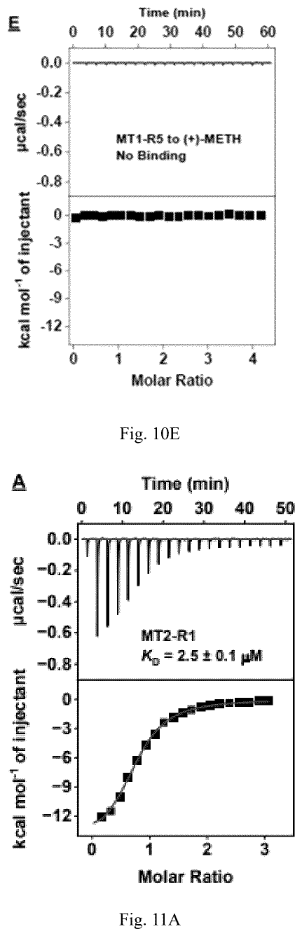

Having established the ineffectiveness of target- and graphene-oxide based SELEX, library-immobilized SELEX was performed to isolate DNA aptamers that bind (+)-methamphetamine. An advantage of this method relative to target-immobilized SELEX is that the target is not immobilized onto a solid support, allowing the aptamer and target to interact freely without masking functional groups on the target. The library is instead immobilized onto streptavidin-immobilized agarose beads via a biotinylated cDNA strand hybridized to the aptamer. Aptamers that bind the target dissociate from the cDNA and are released from the beads, after which they are amplified by PCR and used for the next round of selection (FIG. 3A). SELEX was performed using a 73-nt stem-loop DNA library (FIG. 3B) containing an 9-bp stem and a 30-nt random loop in buffer mimicking physiological conditions (1× PBS with 1 mM MgCl₂). As the target, (+)-methamphetamine was used, which is the more pharmacologically potent of the two enantiomers. For the first five rounds, 100 μM (+)-methamphetamine was used, which was reduced to 50 μM thereafter. In the second round, counter-SELEX was initiated against a variety of interferents (FIG. 9) including ligands known to bind to three-way-junction structured oligonucleotides (e.g., procaine, lidocaine, and quinine); closely related analogs such as 4-hydroxymethamphetamine (4-HMA), amphetamine and 3,4-methylenedioxymethamphetamine (MDMA); other psychoactive drugs (e.g., heroin, fentanyl, and cocaine); structurally similar molecules (e.g., dopamine, bupropion, norepinephrine, and ephedrine); and endogenous compounds (e.g., serotonin, tyramine, tyrosine, and phenylalanine). The progress of SELEX was monitored by collecting aliquots of all eluents from washes with buffer, counter-target, or target, and performing polyacrylamide gel electrophoresis (PAGE) to quantify DNA in these aliquots. Monitoring pool eluted by target every round helps to determine whether target-binding aptamers are being enriched. Typically, as rounds progress, aptamers become more prevalent in enriched pools, and target-induced pool elution should increase. However, throughout the entirety of this trial, consistently low pool elution by (+)-methamphetamine (1-2%) was observed, even after 19 rounds (FIG. 3C). It was also observed that the Round 19 pool had no meaningful affinity for (+)-methamphetamine in a gel-elution assay (FIG. 3D). To investigate this apparent failure more closely, high-throughput sequencing (HTS) of the SELEX pools from this trial was performed. The proportion of unique sequences did not change significantly between Rounds 13 and 19 (˜42%), indicating a lack of aptamer enrichment (Table 6). Five top-ranked aptamer candidates were synthesized for individual affinity characterization with an abundance>0.08% in Round 19 and an enrichment-fold>10 between Rounds 13 and 19 (FIG. 3E). ITC indicated that none of these aptamers displayed measurable target affinity (FIG. 3F, Table 7, Trial 1, and FIGS. 10A-10E), clearly showing that this selection trial was unsuccessful.

### Second Trial of SELEX: Eliminating Counter-SELEX

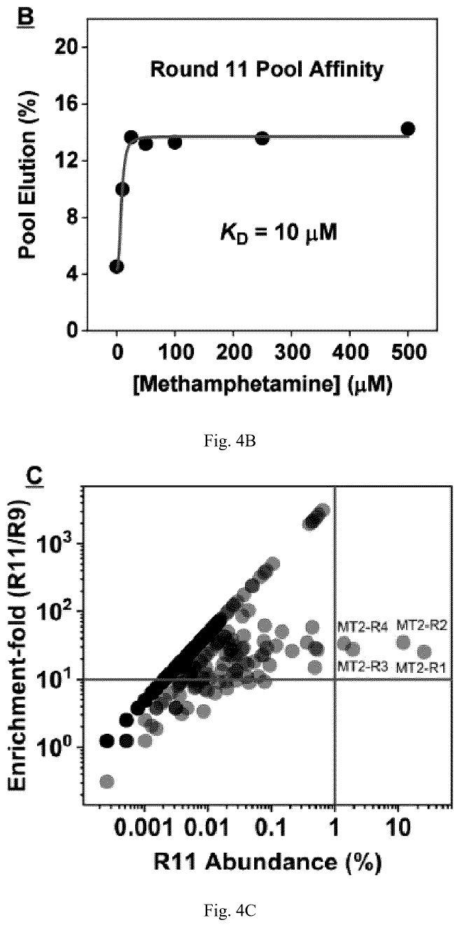

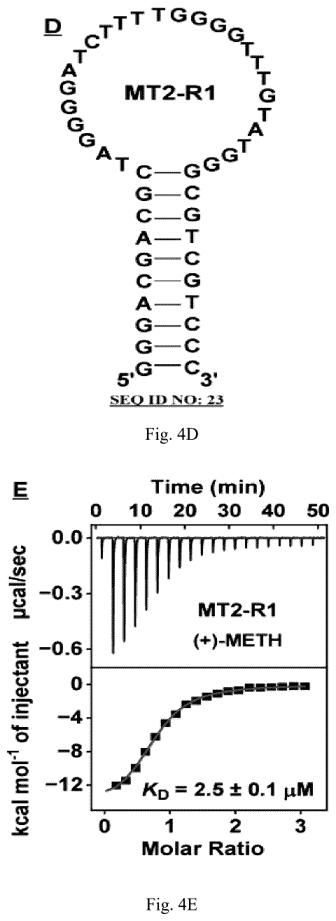

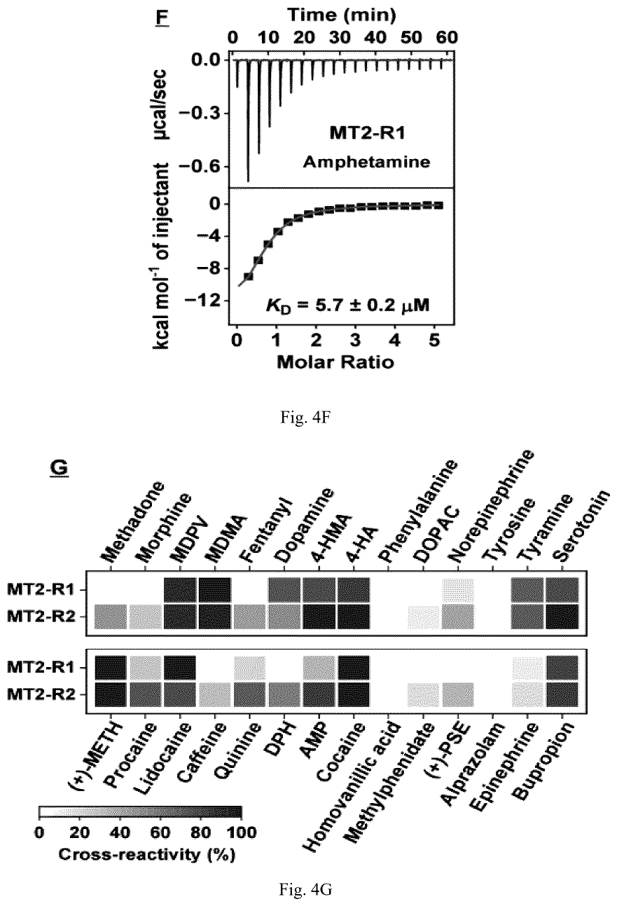

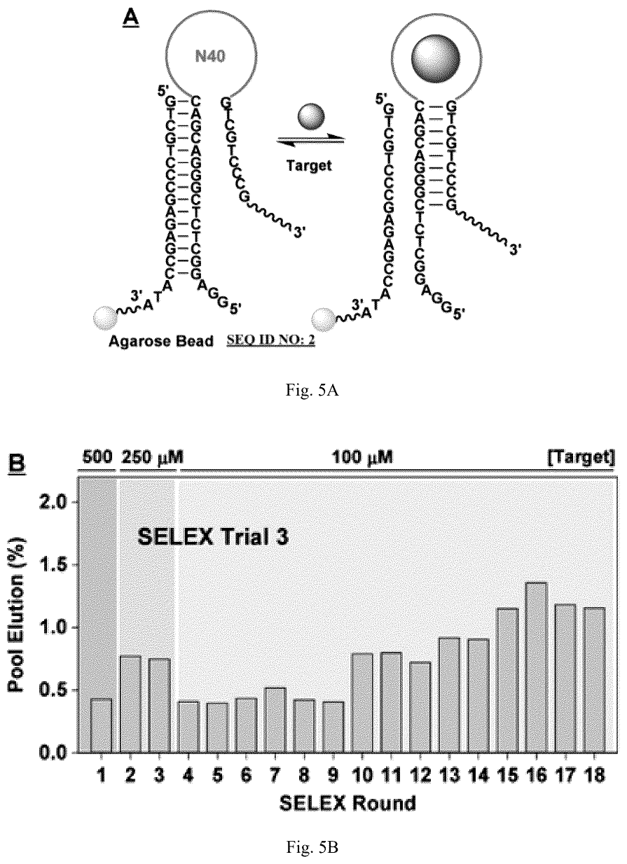

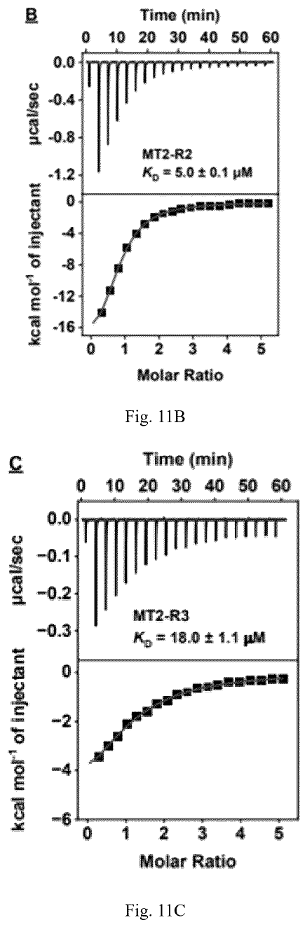

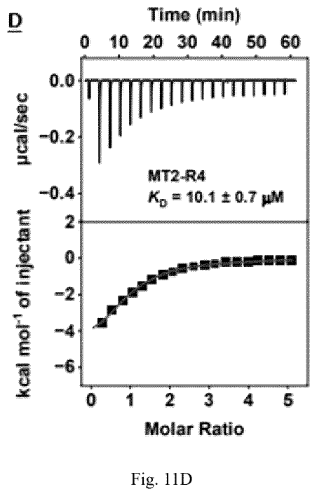

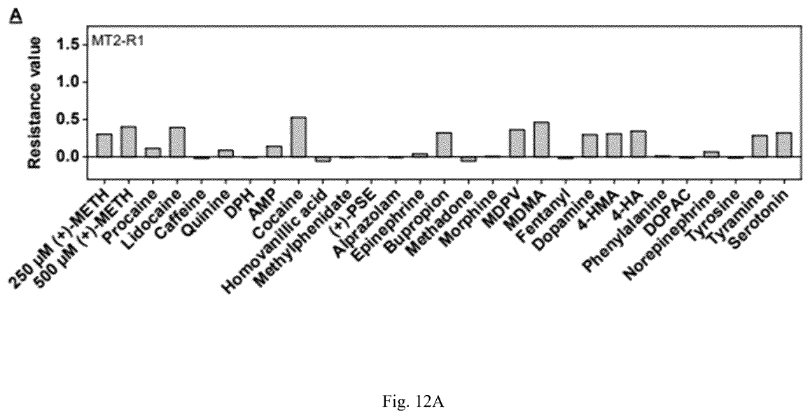

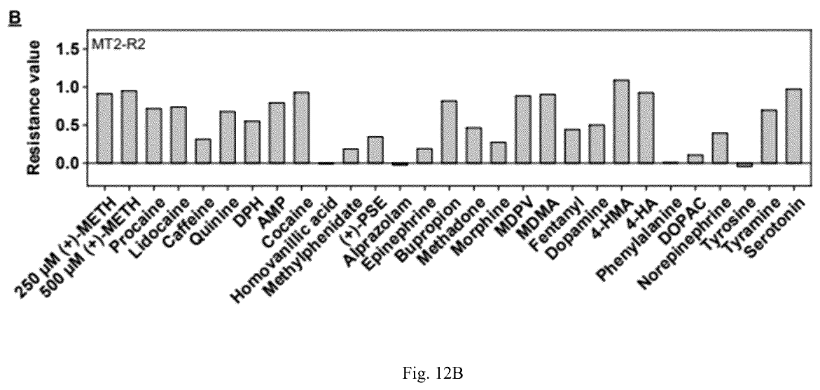

These failures to select aptamers for (+)-methamphetamine suggested that it may not be possible to isolate oligonucleotides for this target. However, it was instead hypothesized that viable methamphetamine aptamers in the library were being removed by the very stringent counter-selection against structurally similar interferents like amphetamine. To determine whether this was true, a second, lower-stringency trial of SELEX was performed, in which the same N30 library as the first trial was used, but counter-SELEX was omitted. From Rounds 1-6, the quantity of pool eluted by (+)-methamphetamine slowly rose from 0.5% to 4%; by Round 11, pool elution reached nearly 5% (FIG. 4A). A gel-elution assay was performed to determine the affinity of the Round 11 pool, and a K_{D} of 10 μM was obtained, with a maximal pool elution of 17% at 25 μM (+)-methamphetamine (FIG. 4B). Having apparently enriched binders to (+)-methamphetamine, the Round 9 and 11 pools were subjected to HTS to identify enriched aptamers. The proportion of unique sequences decreased from 39% in Round 9 to 22% in Round 11, which is a sign of aptamer enrichment (Table 9). Four top-ranked aptamers with Round 11 abundance>1% and enrichment-fold>10 between Rounds 9 and 11 were synthesized (FIG. 4C, Table 9) and their affinity for (+)-methamphetamine was determined using ITC. These aptamers had micromolar affinities, with K_{D} ranging between 2.5-18 μM (Table 7, Trial 2 and FIGS. 11A-11D), which indicated that oligonucleotides can indeed interact with (+)-methamphetamine with relatively good affinity. Unfortunately, the exonuclease assay showed that these aptamers had poor specificity. For instance, the highest affinity aptamer (MT2-R1) had a K_{D} of 2.5±0.1 μM for (+)-methamphetamine (FIG. 4E) but also had a K_{D} of 5.7±0.2 μM for amphetamine (FIG. 4E) and showed similar affinity for a wide range of other structurally similar molecules including lidocaine, cocaine, bupropion, MDPV, MDMA, dopamine, tyramine, and serotonin (FIG. 4G and FIGS. 12A and 12B). The second most abundant aptamer, MT2-R2, likewise had poor specificity. These results indicate that these aptamers generally bind to compounds featuring both an aromatic moiety and an amino group. Therefore, while the feasibility of isolating aptamers for (+)-methamphetamine was confirmed, the poor specificity of these aptamers made them unusable. The SELEX results from Trials 1 and 2 imply that aptamers exhibiting both high affinity and specificity for methamphetamine were either very rare or nonexistent in the employed initial N30 library.

### Third Trial of SELEX: Increasing Library Randomness

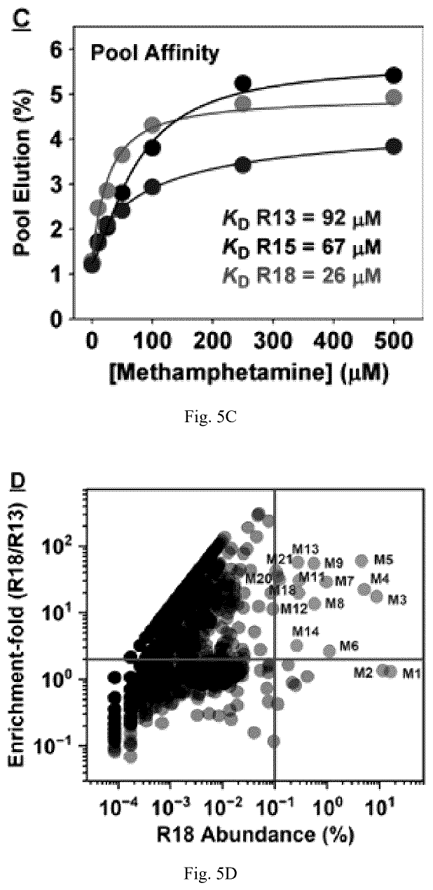

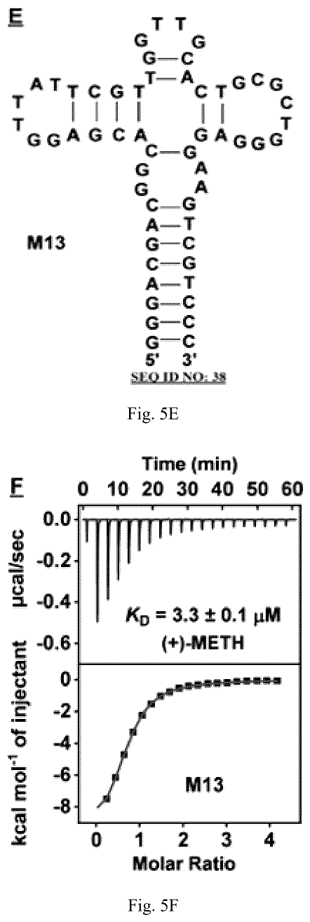

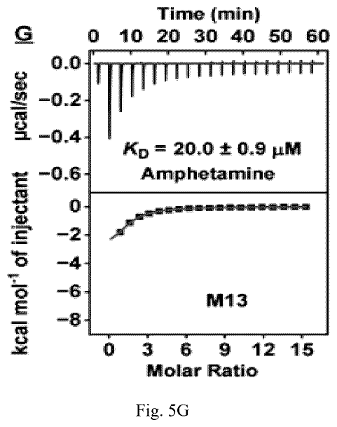

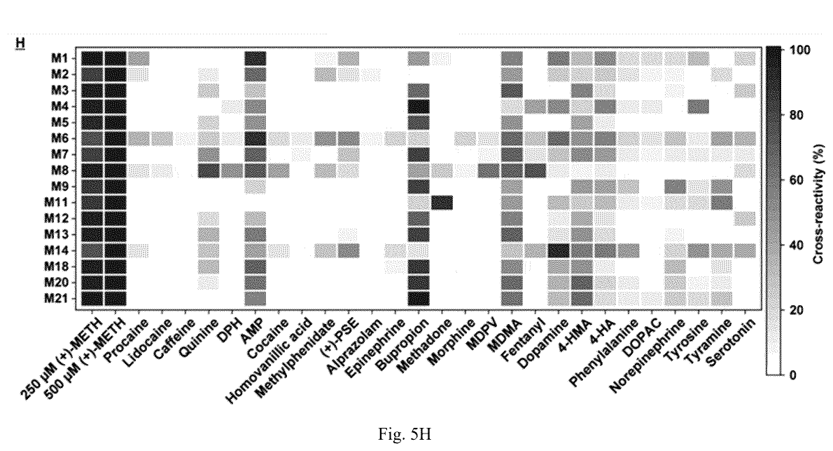

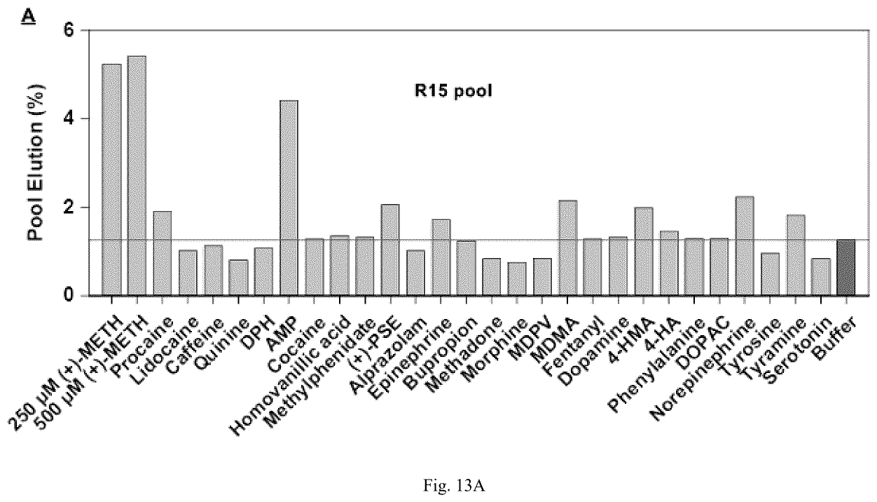

Having established the potential to isolate (+)-methamphetamine-binding aptamers, the next goal was to obtain an aptamer with both high affinity and specificity for this target. However, it was hypothesized that such an aptamer could not be found in an N30 library. This notion was based on two previous studies. The first, performed by the Szostak group, found that longer binding domains play an important role in improved aptamer binding performance (Carothers et al., Informational Complexity and Functional Activity of RNA Structures. J. Am. Chem. Soc. 2004, 126 (16), 5130-5137). In the second study, the Stojanovic group successfully isolated aptamers for the small molecule γ-aminobutyric acid (GABA) using an N44 library after previously failing to do so with an N36 library. These reports suggested that a larger, higher-complexity binding pocket may be necessary for high-performance molecular recognition. Hence, for the third trial of SELEX, a new N40 stem-loop library was used (FIG. 5A). From Rounds 1 to 7, a relatively static pool elution ranging between 0.4-0.7% was observed (FIG. 4B). Again, counter-SELEX from Round 2 onward was employed, and it was noticed that in Round 7 a sizable portion of the library was eluted by procaine (1.5%), lidocaine (2.4%), diphenhydramine (1.4%), tyramine (1.2%), methylphenidate (1%), alprazolam (1.3%), bupropion (1.5%), and 4-hydroxymethamphetamine (4-HMA) (0.8%). This was expected, as all these molecules have a close resemblance to (+)-methamphetamine, with the exception of alprazolam. In Round 8, additional counter-SELEX washes for structurally similar molecules such as amphetamine, 4-HMA, norepinephrine, bupropion, and MDMA, were performed and increased pool elution in turn for these interferents was observed. In Rounds 8 and 9, pool elution by (+)-methamphetamine remained stagnant at 0.4% (FIG. 5B). However, in Round 10, target-induced pool elution doubled to 0.8% and remained similarly high in Rounds 11 and 12. This suggested that although the counter-SELEX stringency was relatively high, specific aptamers for (+)-methamphetamine could potentially continue to be enriched. Notably, in Round 13, target-induced pool elution increased again to 0.9% (FIG. 4B), and elution by counter-targets such as diphenhydramine, 4-HMA, tyramine, and dopamine was reduced by half relative to previous rounds. This shift in the binding properties of the pool may indicate a change in the abundance of different aptamers in the pool. However, while pool elution rose again to 1.2% for (+)-methamphetamine in Round 15, a sizable increase in pool elution by amphetamine (10%) was observed. The gel-elution assay was performed for the Round 13 and 15 pools to determine binding affinity to (+)-methamphetamine and specificity against the counter-targets. These pools bound to (+)-methamphetamine with a K_{D} of 92 and 67 μM with maximal pool elution of 3% and 5% at 500 μM (+)-methamphetamine, respectively (FIG. 5C). However, the Round 15 pool responded to amphetamine, procaine, 4-HMA, tyramine, pseudoephedrine, norepinephrine, bupropion, and MDMA with cross-reactivity>30% relative to 500 μM target (FIG. 13A). While pool elution by target rose to 1.4% in Round 16, amphetamine eluted 12% of the pool, and this pattern and level of elution continued in Rounds 17 and 18 (FIG. 5B). A gel-elution assay for the Round 18 pool revealed an improved K_{D} of 26 μM for (+)-methamphetamine (FIG. 5C), however, the pool also responded to several counter-targets, such as amphetamine, with cross-reactivity>40% relative to 500 μM (+)-methamphetamine (FIG. 13B). Since pool specificity was not improving in these later selection rounds, this trial of SELEX was ended.

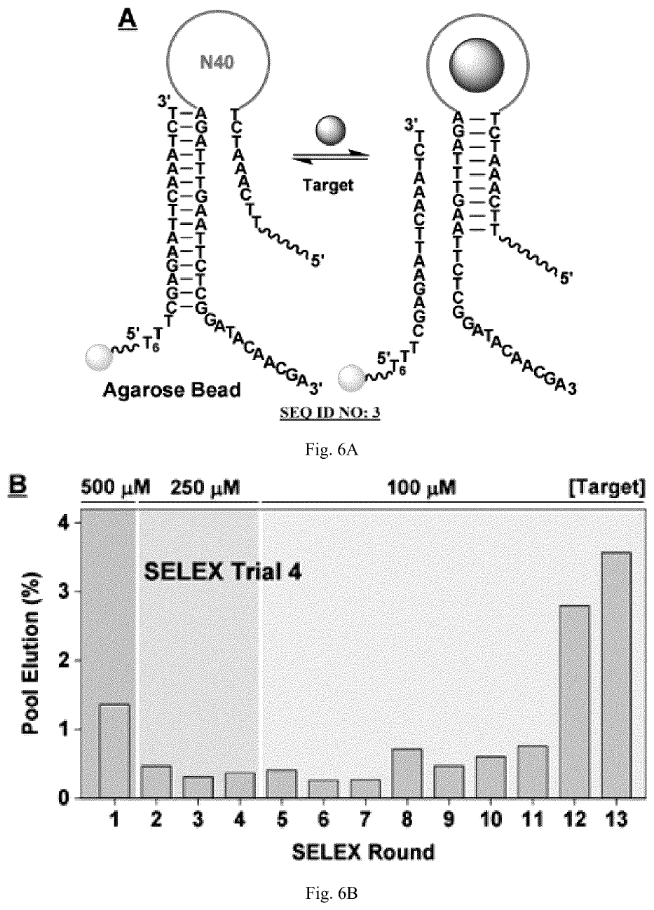

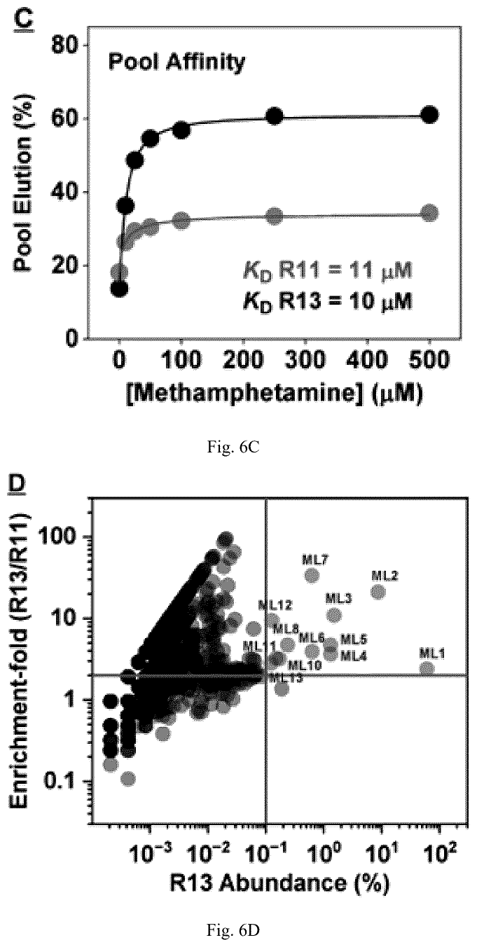

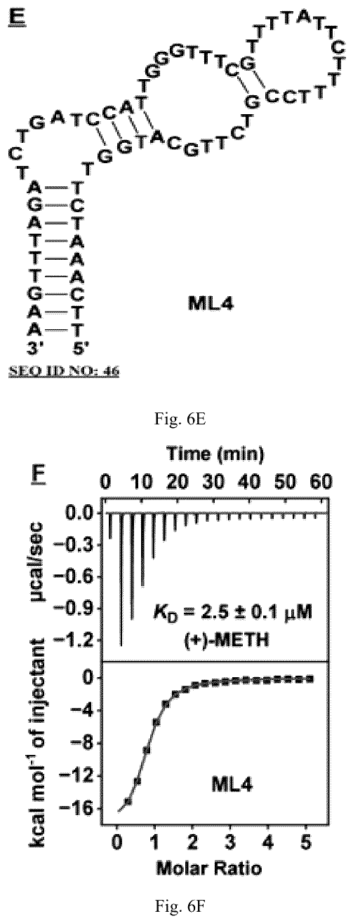

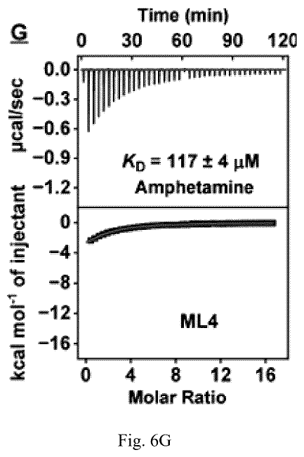

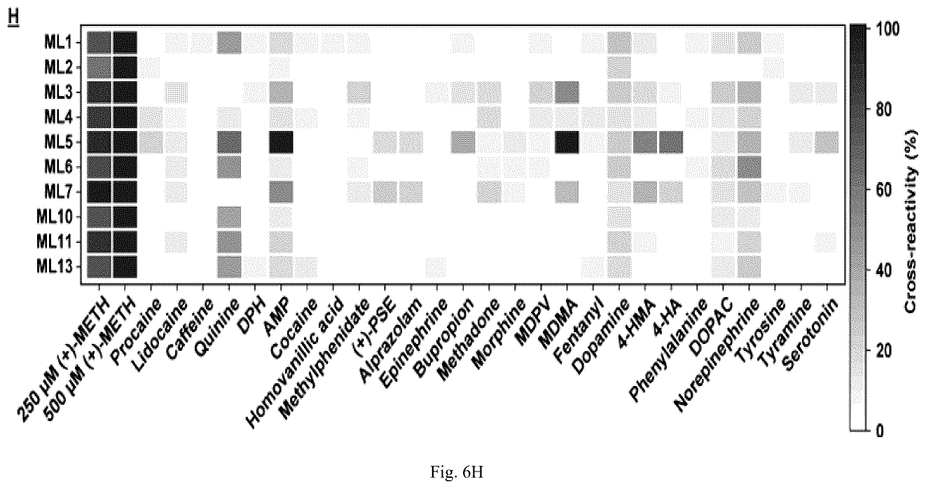

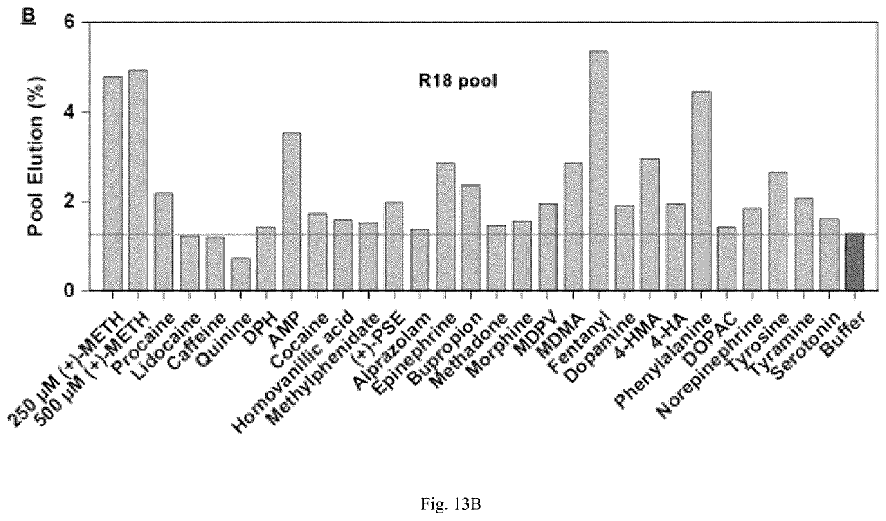

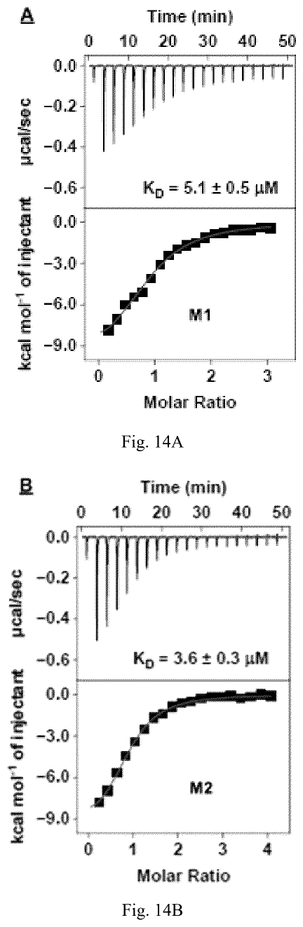

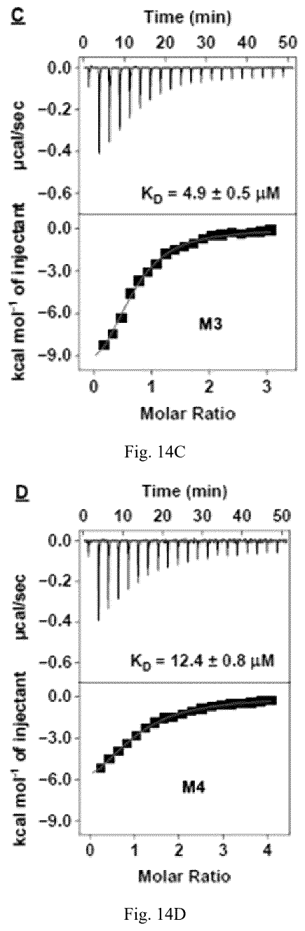

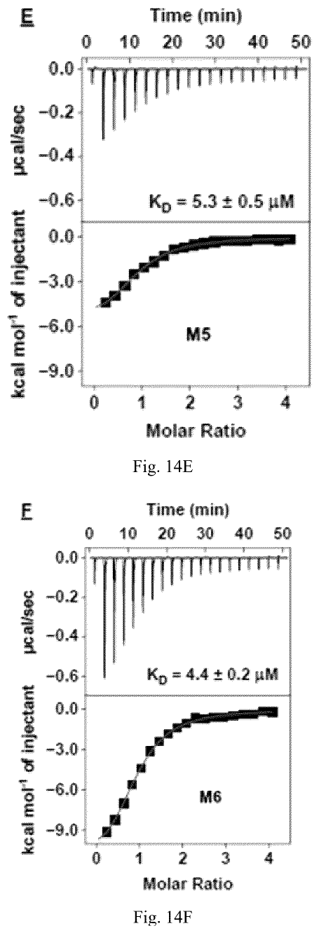

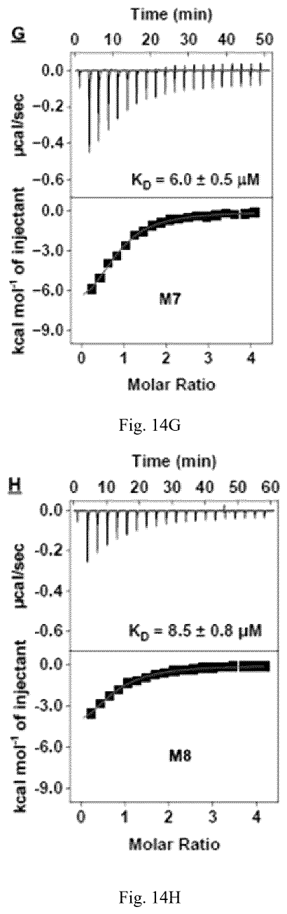

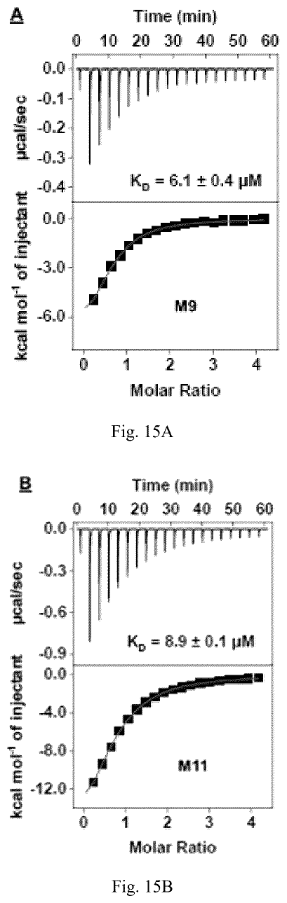

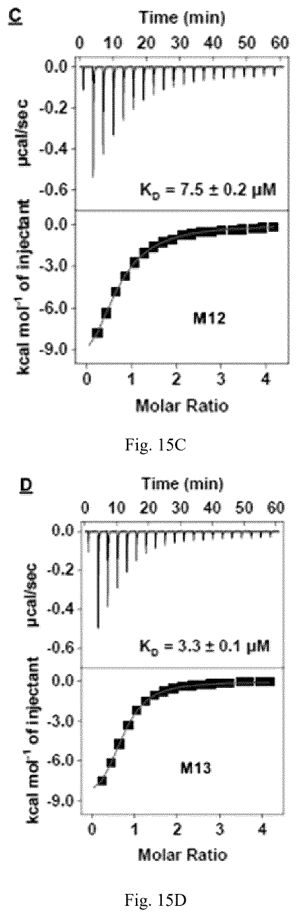

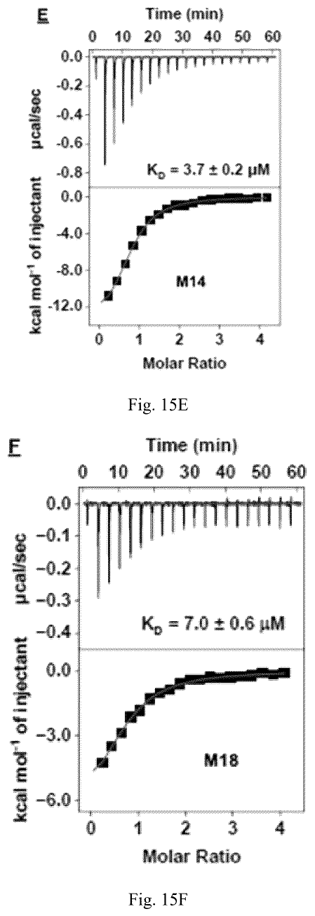

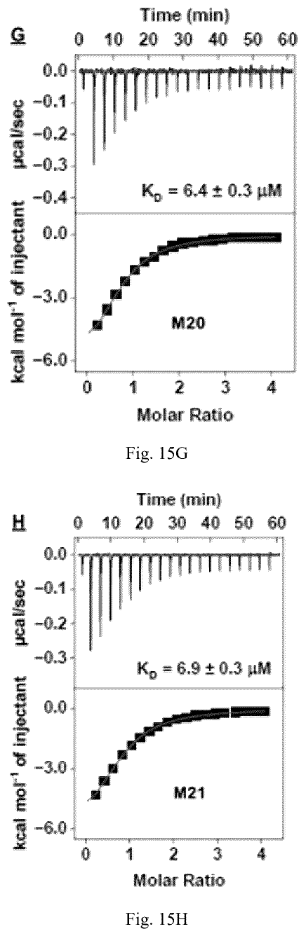

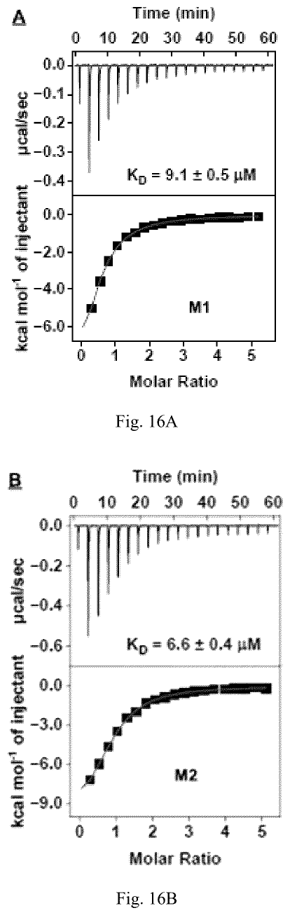

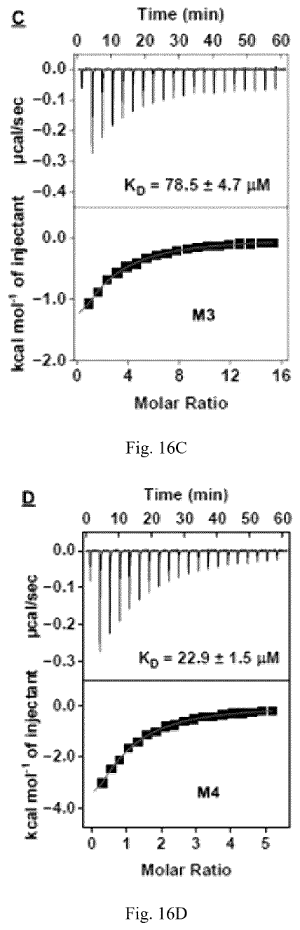

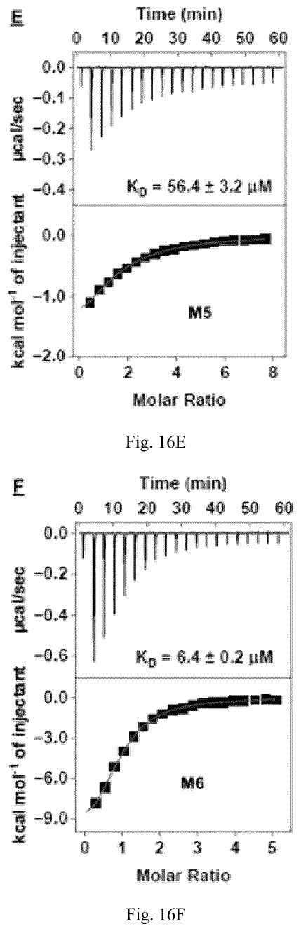

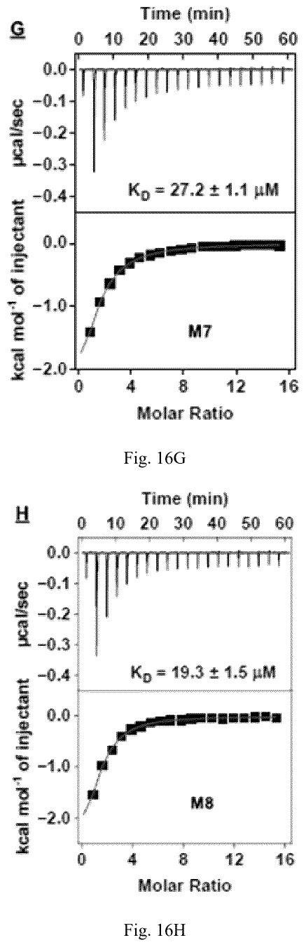

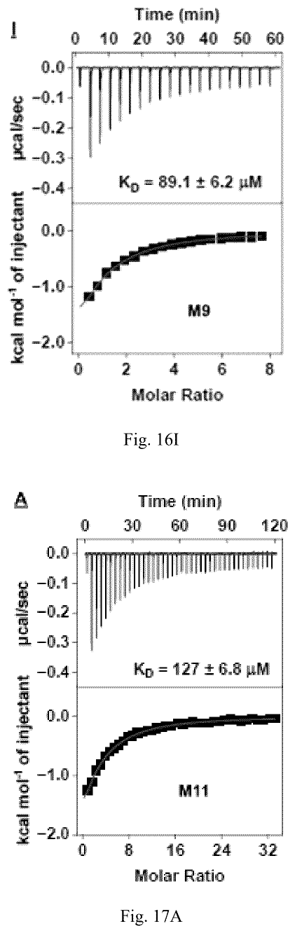

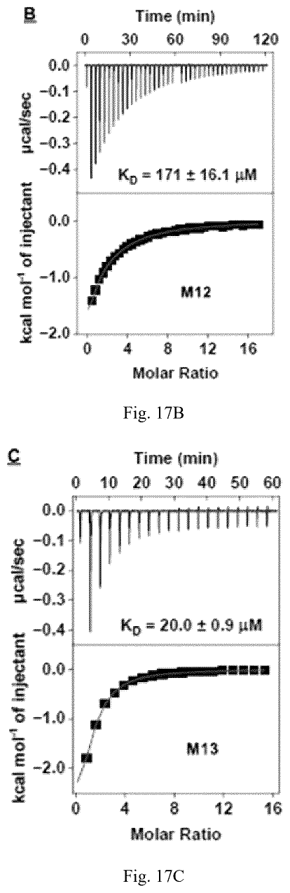

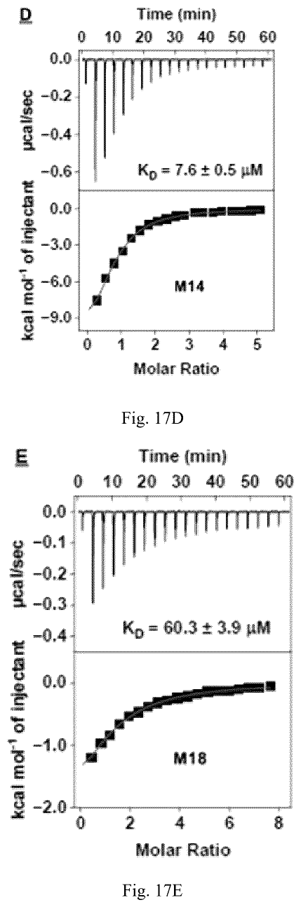

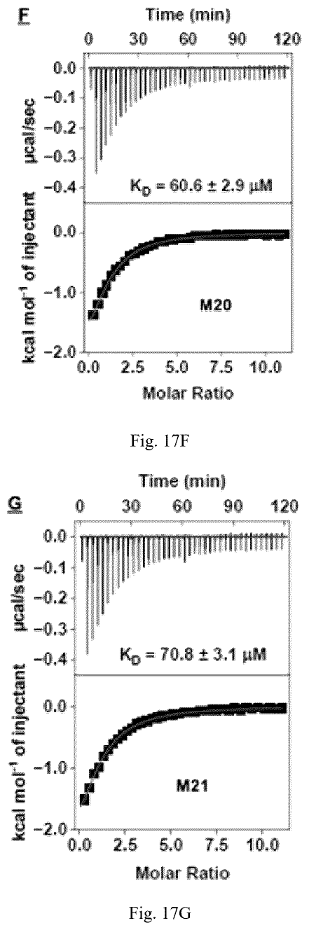

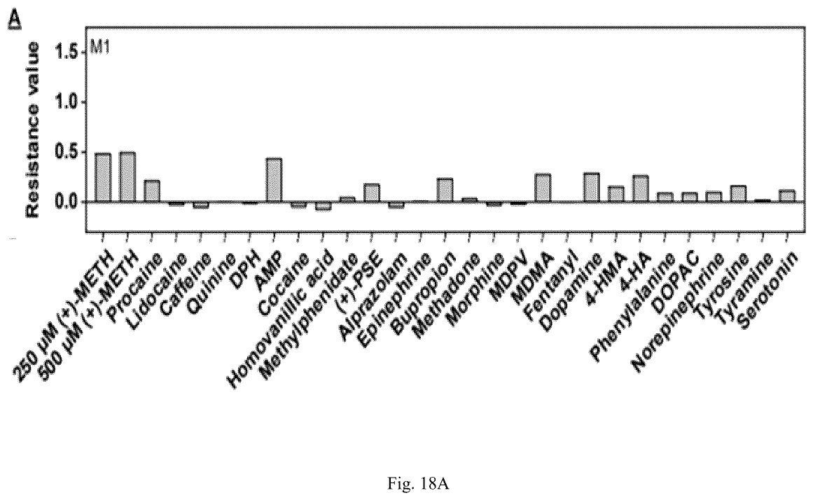

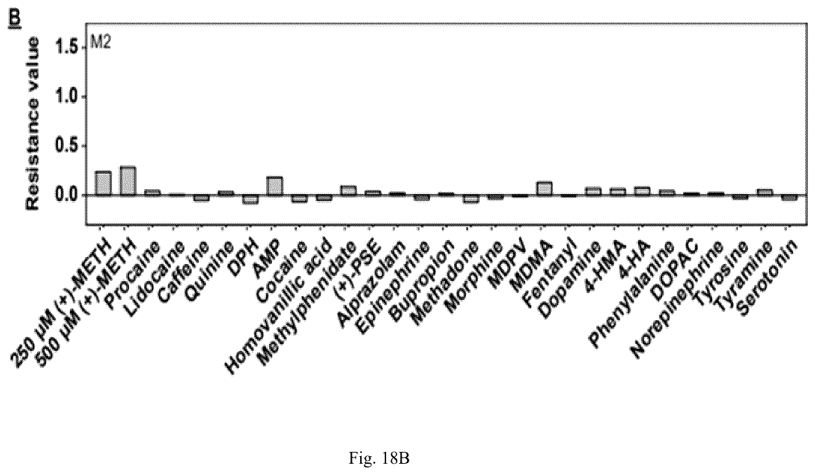

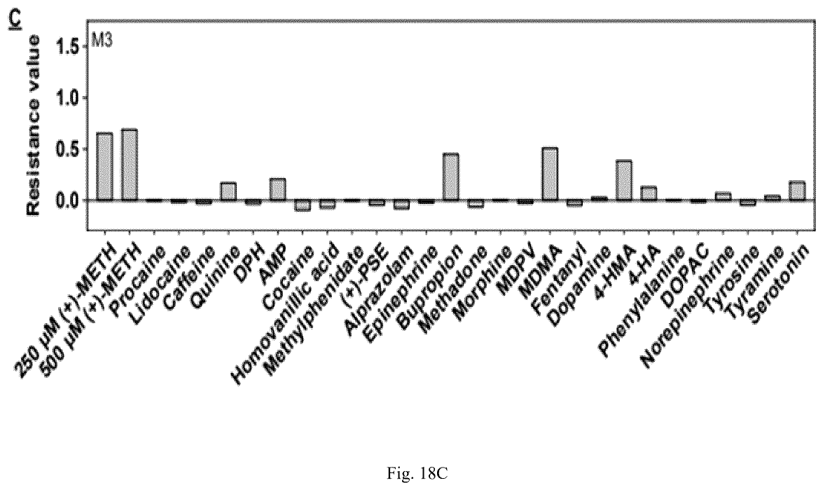

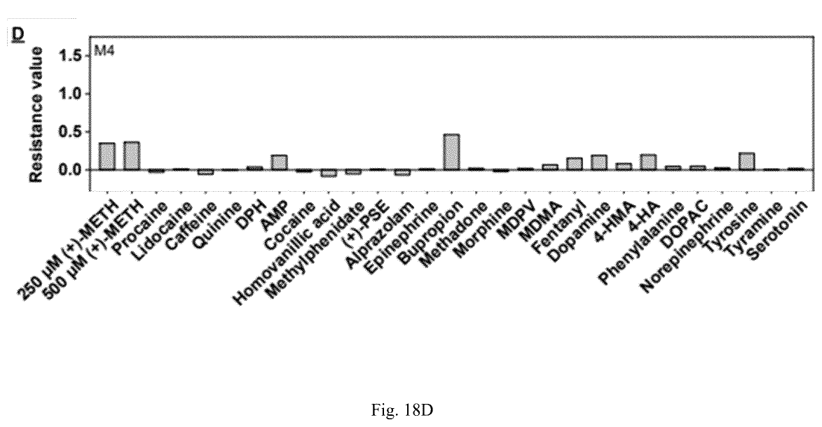

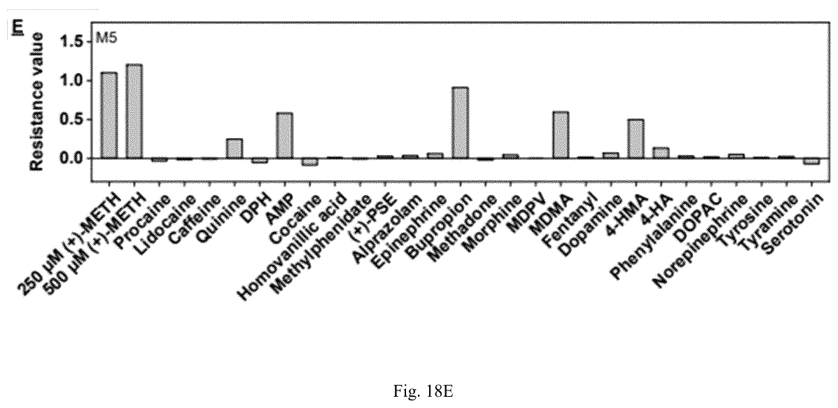

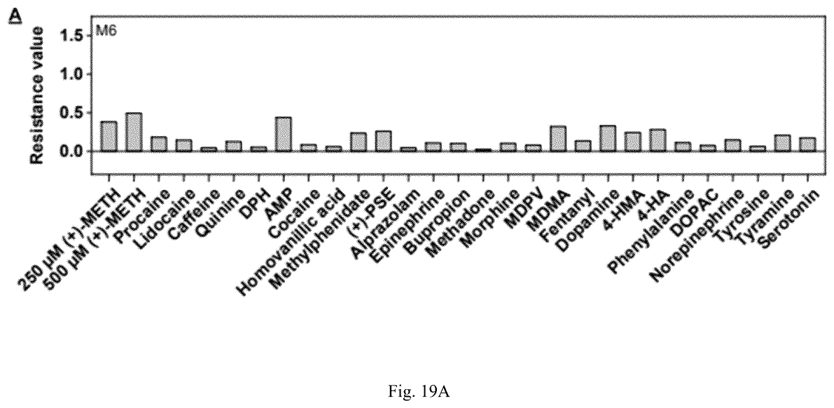

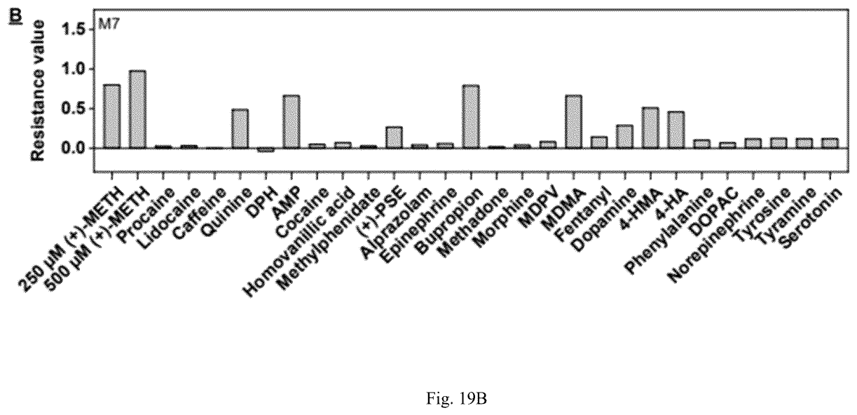

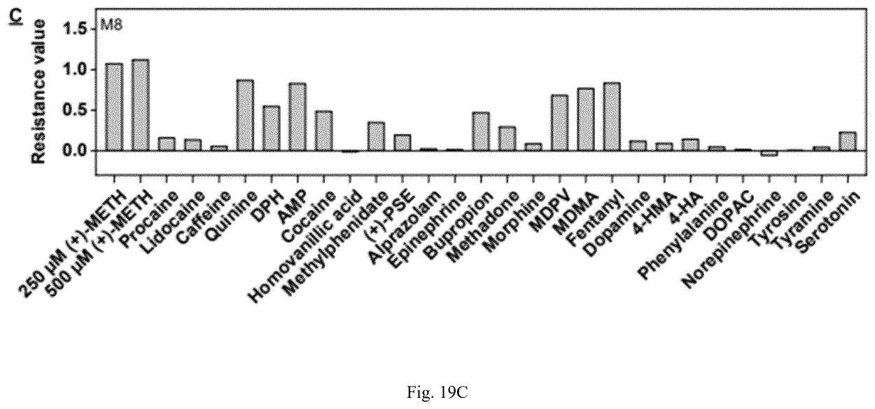

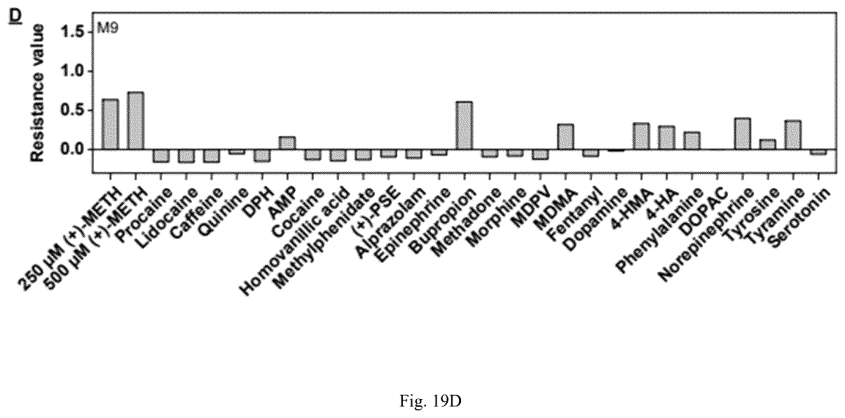

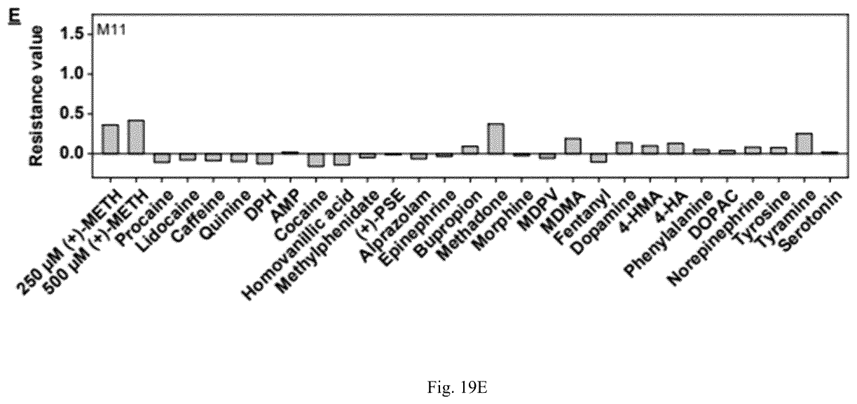

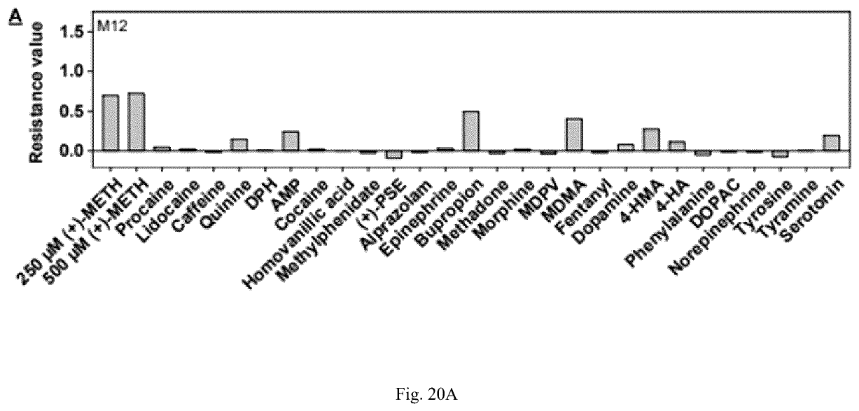

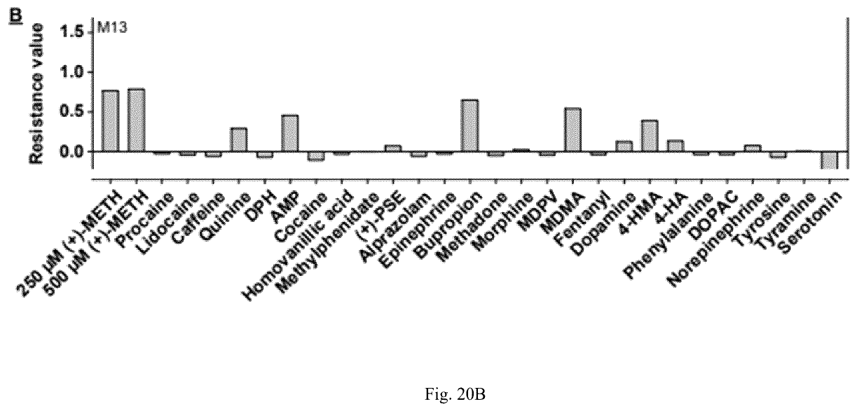

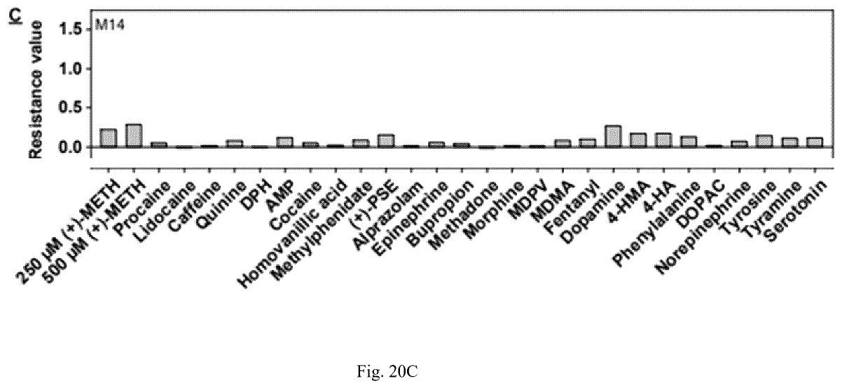

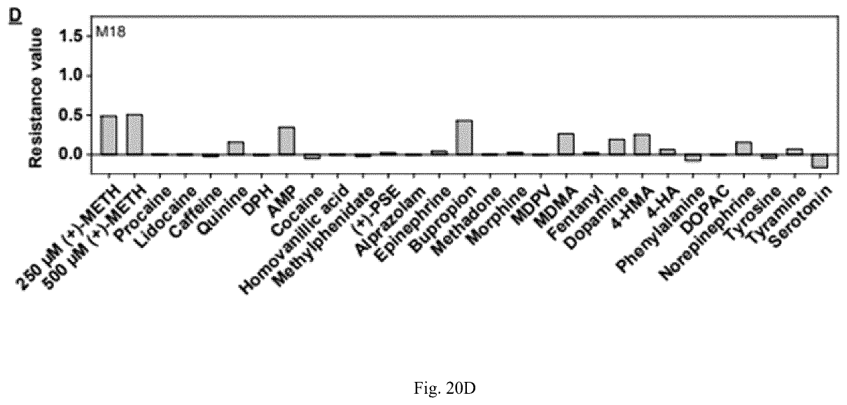

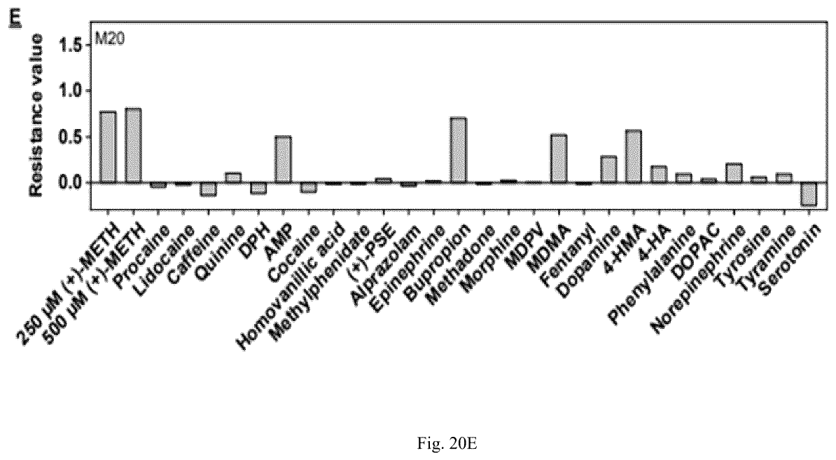

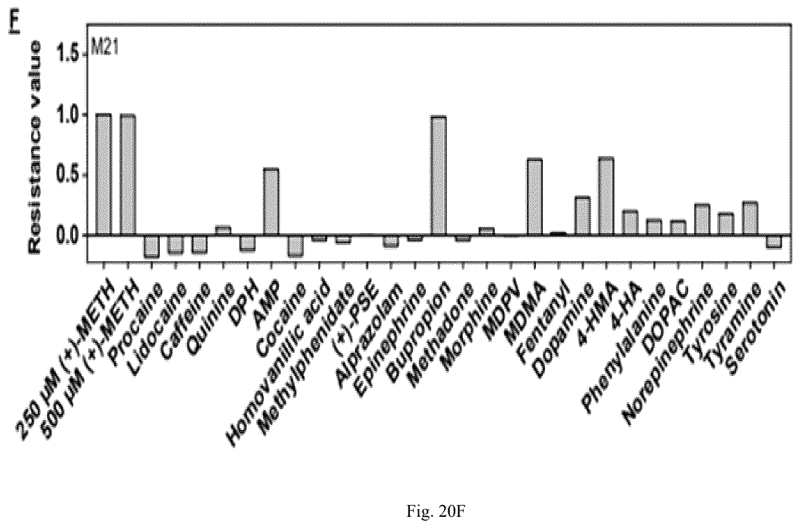

The SELEX pools from this trial were subjected to HTS. The percentage of unique sequences dropped from 46% to 31% between Rounds 7 and 13, and further decreased to 19% in Round 18, indicating that the pools were being enriched (Table 6). To select aptamer candidates for binding characterization, aptamers with Round 18 abundance>0.1% and enrichment-fold>2 between Rounds 13 and 18, as well as the two most abundant sequences in the Round 18 pool were synthesized (FIG. 5D, Table 9). ITC was performed to determine the affinity of 16 different aptamers and K_{D}s ranging between 3.3-12.4 μM for (+)-methamphetamine (Table 7, FIGS. 14A-14H and 15A-15H) and 6.4 to 171 μM for amphetamine (Table 7 and FIGS. 16A-16I and 17A-17G) were obtained. Therefore, the affinities of these aptamers were not meaningfully different from those discovered in the N30 pool, although the highest-affinity aptamer, M13 (FIG. 5E), had 6-fold higher binding affinity for (+)-methamphetamine relative to amphetamine (FIGS. 5F-5G). Next the specificity of 11 of these aptamers was assessed using the exonuclease-based assay, and it was determined that essentially all aptamers had poor to moderate specificity, with cross-reactivity mainly apparent with amphetamine, quinine, bupropion, MDMA, 4-HMA, norepinephrine, phenylalanine, and dopamine (FIG. 5H, FIGS. 18A-18E, FIGS. 19A-19E, and FIGS. 20A-20F). The aptamer with the best specificity, M4, had a K_{D} of 12 μM for (+)-methamphetamine with minimal cross-reactivity to all counter-targets except for amphetamine, bupropion, tyrosine, dopamine, MDMA, and 4-HMA. This was a notable improvement over the best aptamer from the previous trial of SELEX, which cross-reacted to at least 10 different compounds with similar affinity to (+)-methamphetamine. Therefore, it was concluded that whereas an N40 library yielded aptamers with better specificity than N30 libraries, they were still not sufficiently specific.

### Fourth Trial of SELEX: Altering Buffer Conditions Leads to High-Performance Aptamers

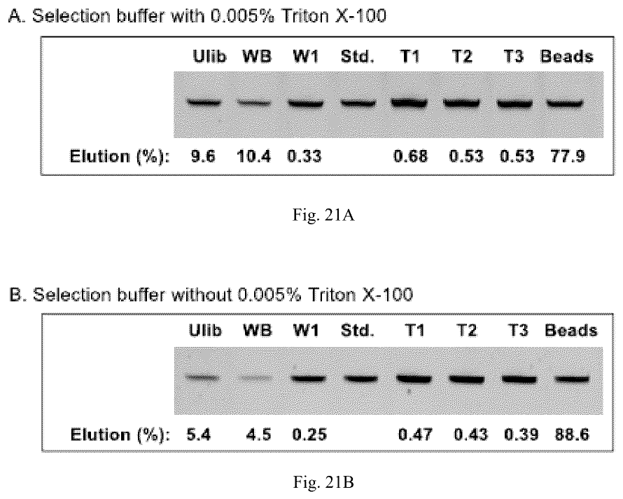

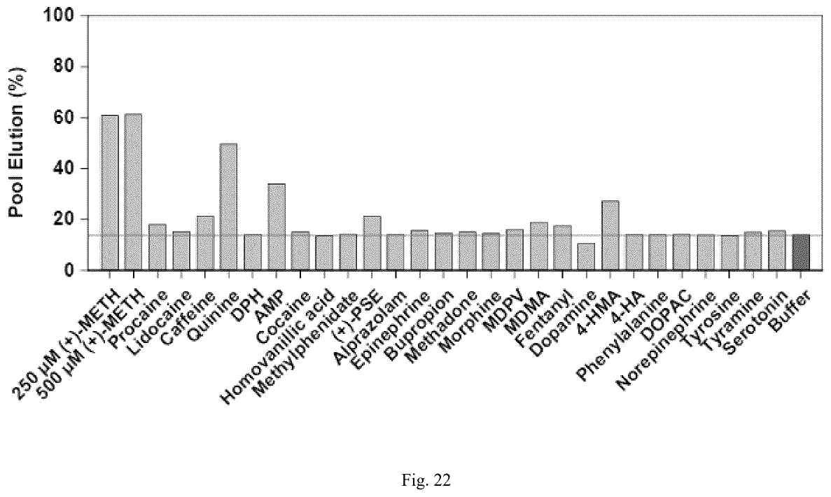

In the final trial of SELEX, it was investigated if changing the composition of the selection buffer could yield higher quality aptamers. A systematic study by Carothers et al. determined that higher concentrations of Mg²⁺ (1 mM versus 5 mM) in the selection buffer led to the enrichment of higher-affinity aptamers. (Carothers et al. 2010) Similarly, it was found in past selection of cocaine aptamers with an N30 library in buffer containing 5 mM Mg²⁺ yielded aptamers with 2.5-fold higher affinity (Alkhamis et al. High-Affinity Aptamers for In Vitro and In Vivo Cocaine Sensing. J. Am. Chem. Soc. 2024, 146 (5), 3230-3240) than cocaine aptamers isolated by the Stojanovic group with an N36 library in buffer containing 2 mM Mg²⁺. For the fourth trial, the concentration of Mg²⁺ was increased from 1 mM to 5 mM. Since 5 mM Mg²⁺ is insoluble in 1× PBS, 0.5× PBS was used instead. In addition, during the negative and counter-selection steps, 0.005% (v/v) Triton was incorporated in the selection buffer based on the presumption that it would increase the separation efficiency with which nonspecific binders are removed from the library. This was supported by testing with the naive N40 library, in which a 2-fold increase in library elution after washing with Triton-containing versus Triton-free buffer was observed (FIGS. 21A-21B). In Round 1, pool elution of the N40 library by target was 1.4% (FIG. 6B). In Round 2, counter-SELEX was initiated, this time including the problematic counter-targets amphetamine, bupropion, and MDMA earlier on and with more washes; then 4-HMA, norepinephrine, epinephrine, dopamine, and tyramine were introduced in Round 3. Pool elution by (+)-methamphetamine hovered between 0.3-0.4% in Rounds 2-7, and as in previous trials, counter-targets closely related to (+)-methamphetamine in structure such as amphetamine, 4-HMA, and MDMA eluted more pool than the target. In Round 8, a shift in the binding properties of the pool was observed, with relatively lower elution by counter-targets in general and a near-doubling of pool elution by (+)-methamphetamine to 0.7%. This general trend continued in Rounds 9-11. In Round 12, pool elution by (+)-methamphetamine surged to 2.8%, with only amphetamine and quinine eluting meaningful proportions (>1.5%) of the pool. Target-induced pool elution increased again in Round 13 to 3.6%, indicating the successful enrichment of (+)-methamphetamine binders. To assess pool binding properties, the gel-elution assay for Rounds 11 and 13 was performed and a K_{D} of 11 and 10 μM for (+)-methamphetamine, respectively, was obtained (FIG. 6C), the highest pool affinity obtained thus far. The specificity of the Round 13 pool was determined using the gel-elution assay and meaningful cross-reactivity only to quinine, 4-HMA, and pseudoephedrine was observed (FIG. 22). Notably, the level of pool elution by amphetamine, dopamine, norepinephrine, epinephrine, and MDMA were similar to buffer alone, indicating a considerable improvement in pool specificity. Therefore, SELEX was concluded at this round.

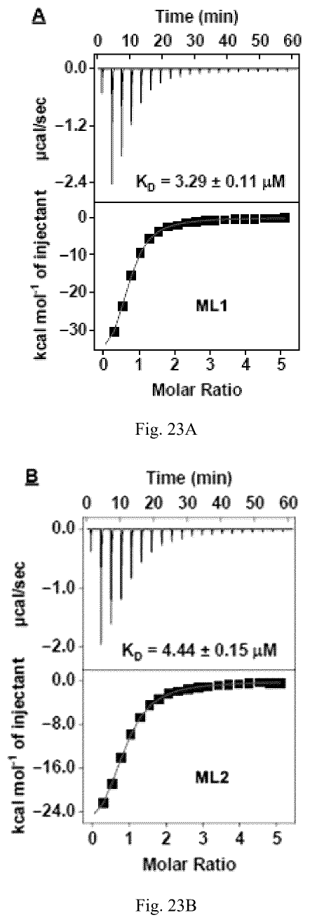

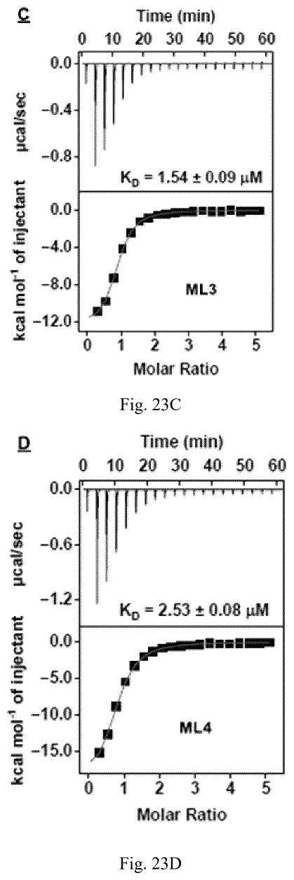

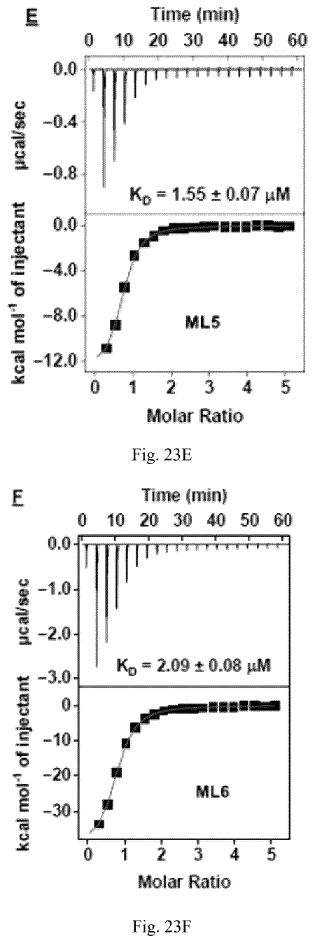

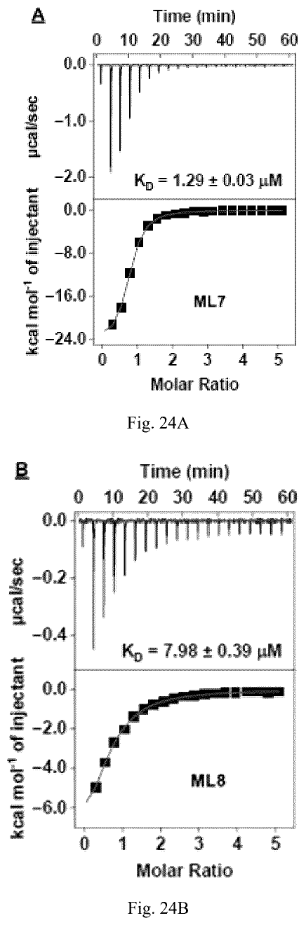

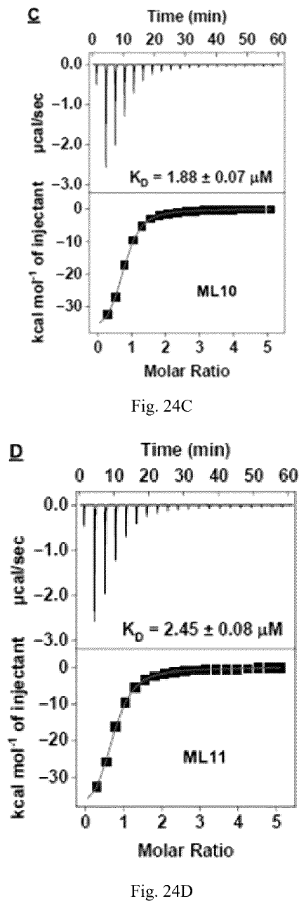

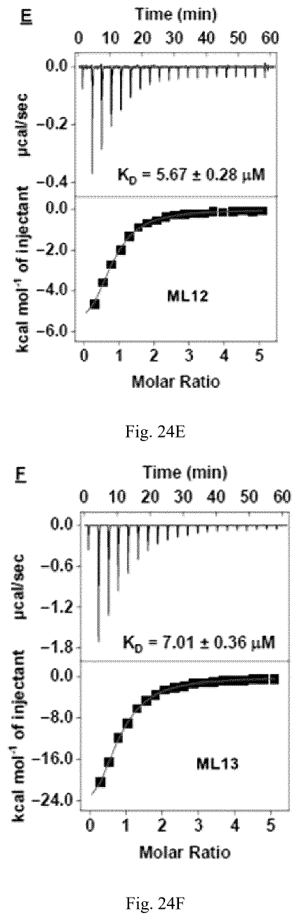

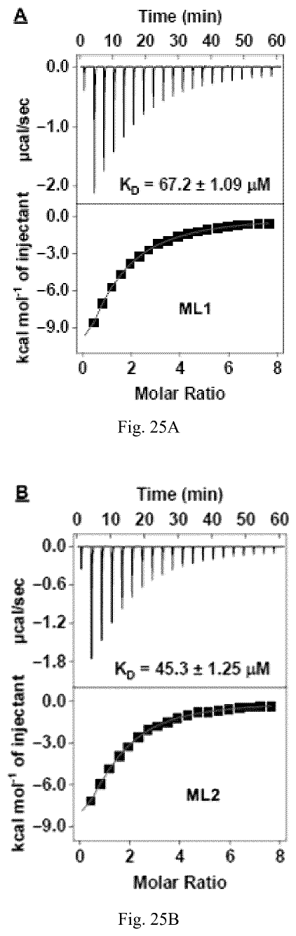

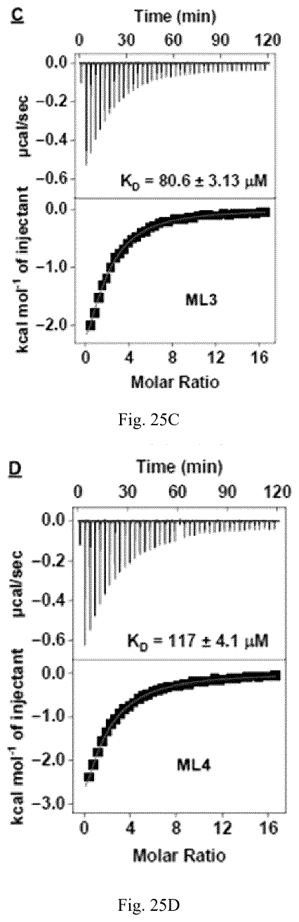

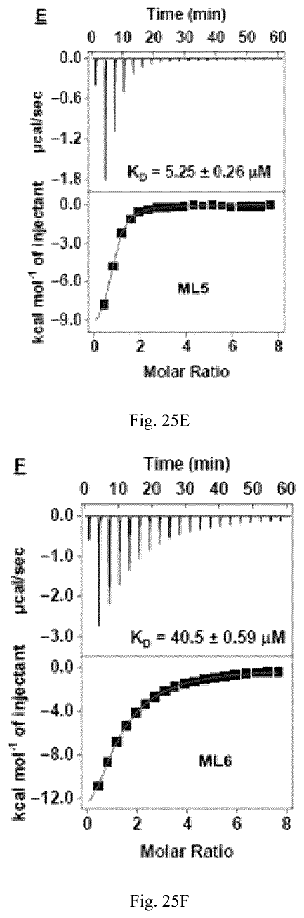

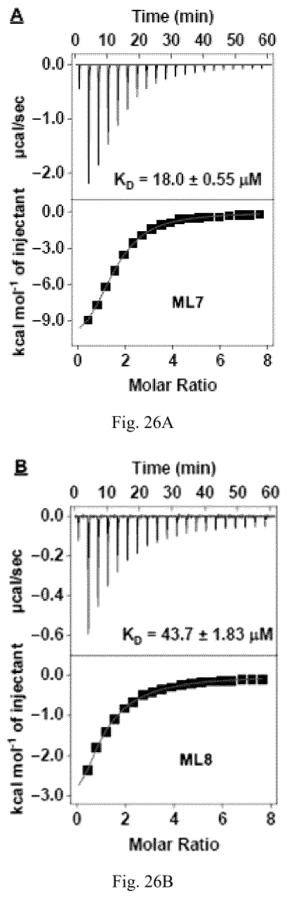

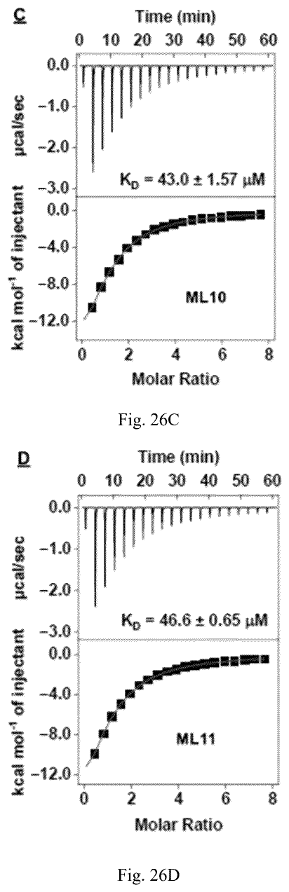

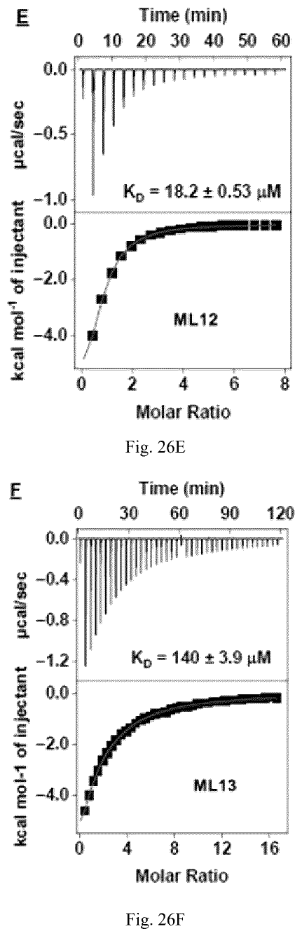

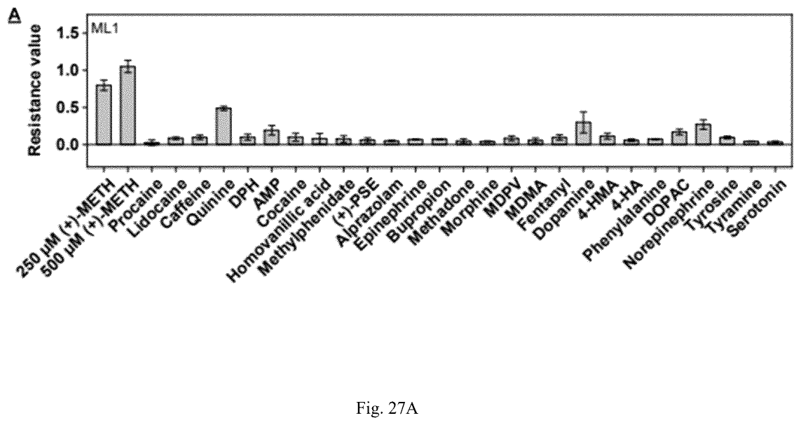

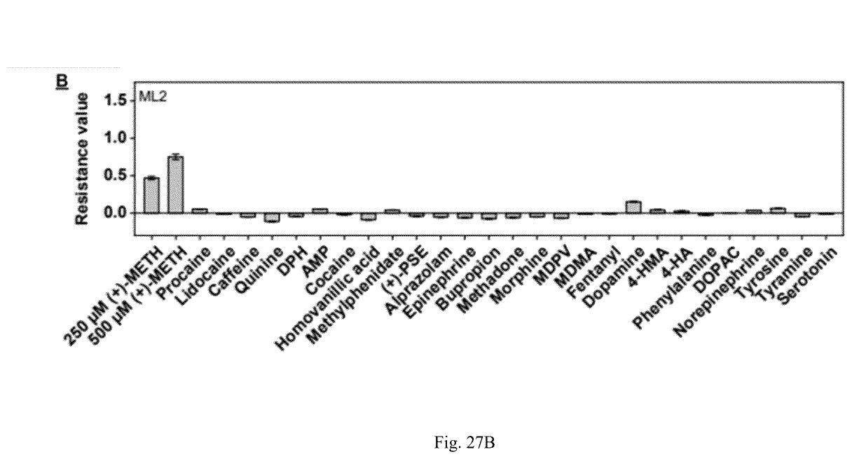

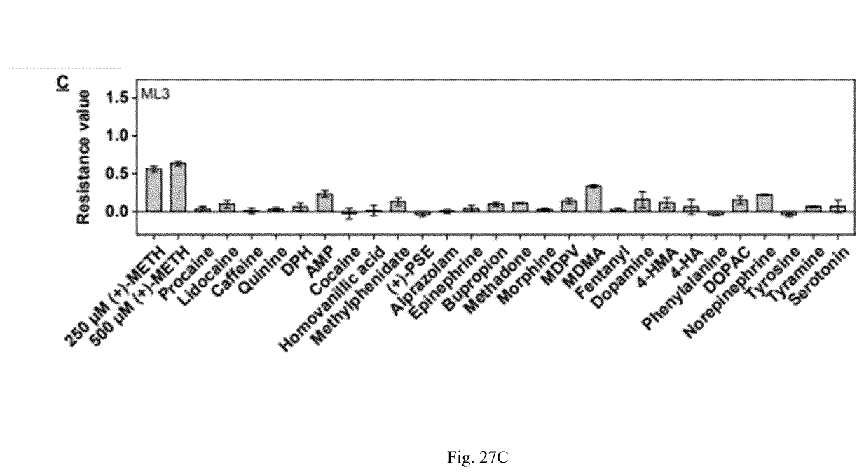

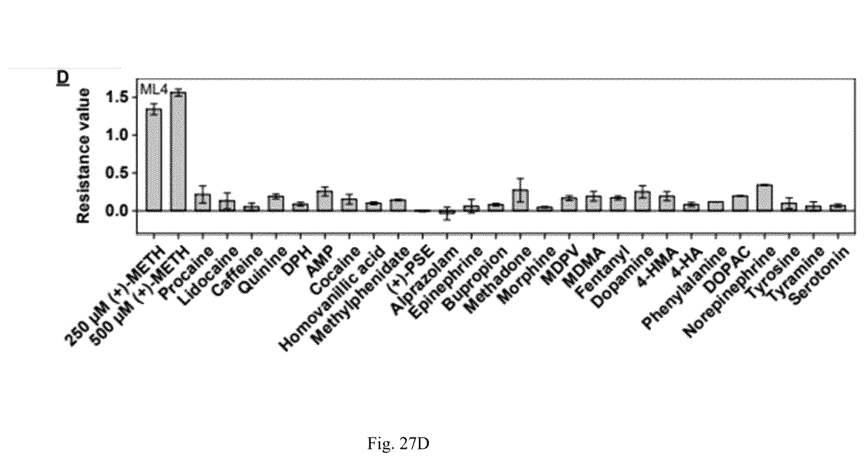

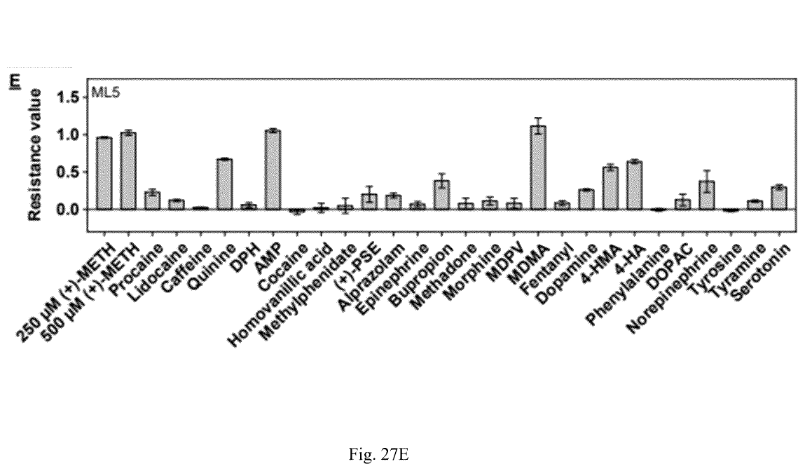

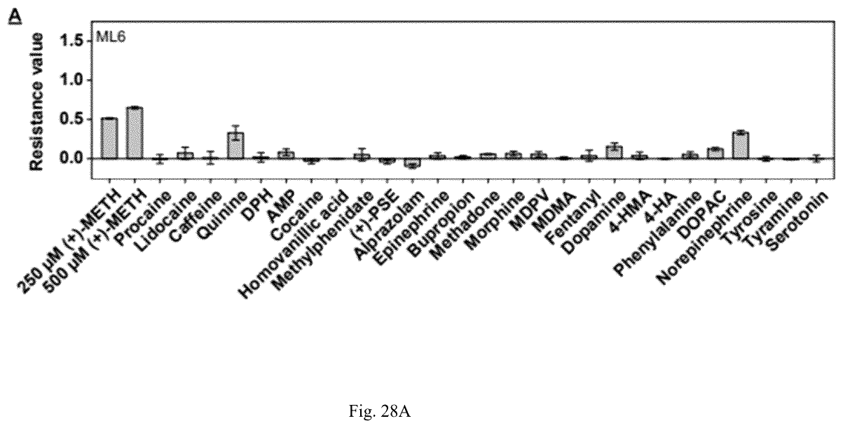

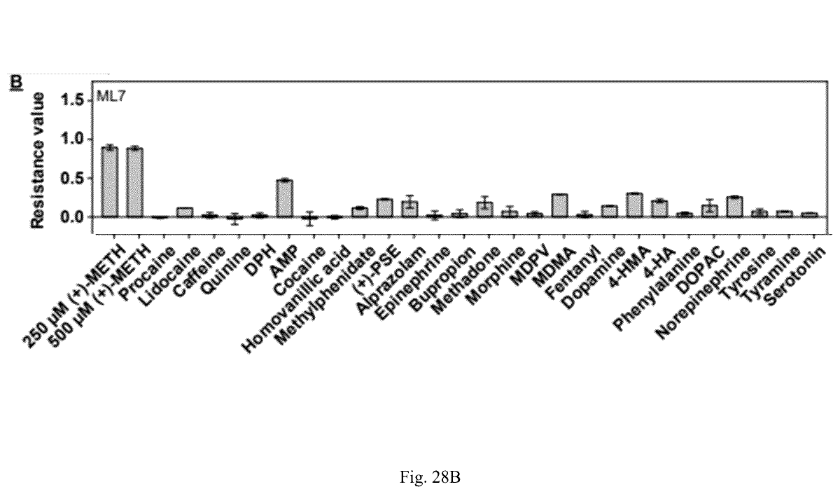

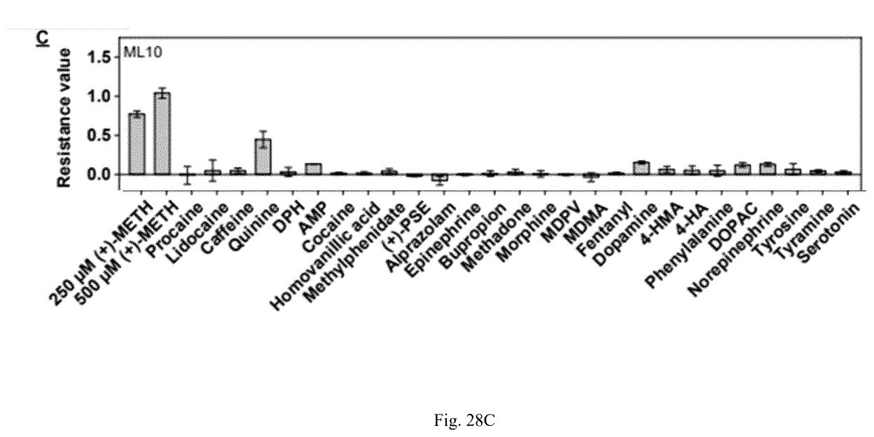

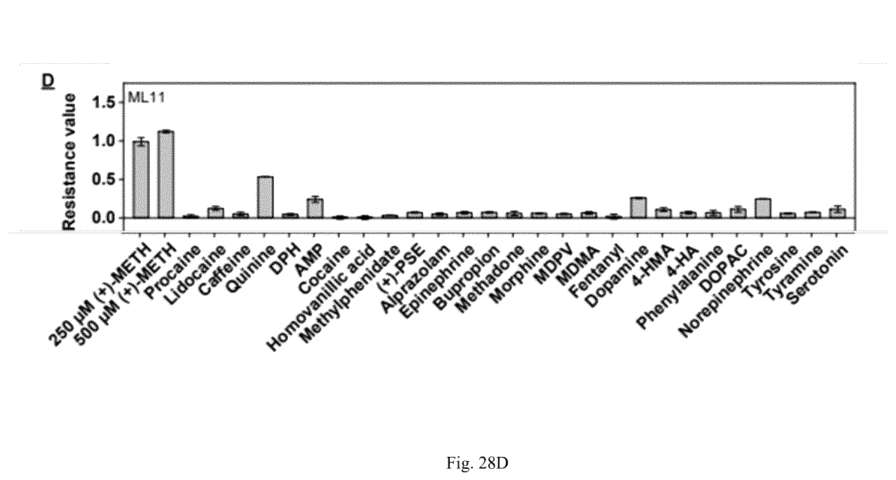

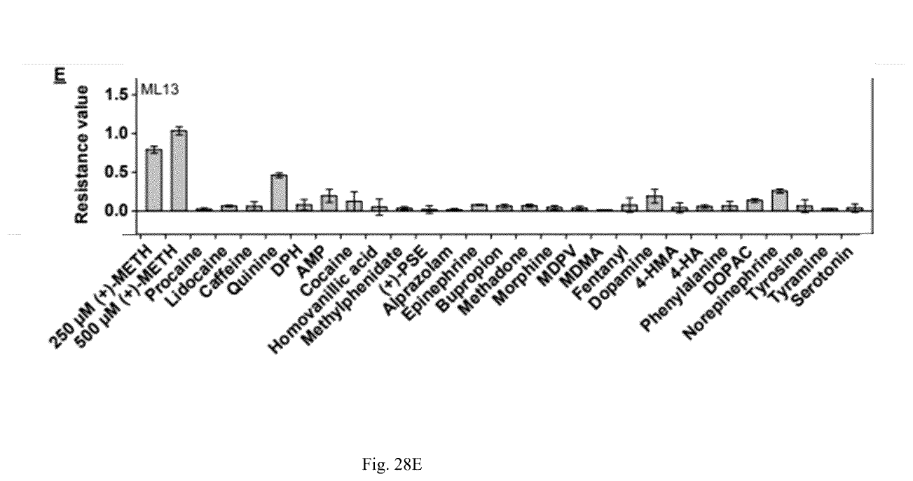

HTS analysis of SELEX pools from this trial confirmed that (+)-methamphetamine-binding aptamers were enriched. The proportion of unique sequences decreased from 44% in Round 9 to 9.6% in Round 13 (Table 6). To select aptamer candidates for further characterization, 13 sequences that had a Round 13 abundance>0.1% and an enrichment-fold>2 between Rounds 11 and 13 were chosen (FIG. 6D, Table 9). Using ITC, K_{D}s for (+)-methamphetamine were determined ranging between 1.3-8 μM (Table 7, FIGS. 23A-23F, and FIGS. 24A-24F), which represents a clear improvement relative to aptamers identified in the previous trials. Their affinity for amphetamine was also determined using ITC, and all had lower affinity compared to (+)-methamphetamine (Table 7, FIGS. 25A-25F, and FIGS. 26A-26F). The most specific aptamer, ML4 (K_{D}=2.5±0.1 μM) (FIGS. 6E-6F), had a 50-fold lower affinity for amphetamine (K_{D}=117±4 μM) (FIG. 6G). Next, the exonuclease fluorescence assay was used to determine aptamer specificity, and in this assay, ML4 did not meaningfully respond to amphetamine, pseudoephedrine, bupropion, MDPV, MDMA, dopamine, phenylalanine, or tyramine, except to 4-HMA with 15% cross-reactivity (FIG. 6H, FIGS. 27A-27E, and FIGS. 28A-28E). More generally, all aptamers had improved specificity relative to those identified from previous trials. The highest-affinity aptamer, ML7 (K_{D}=1.30±0.03 μM), also had high specificity, but had nearly 33% cross-reactivity to 4-HMA and 53% cross-reactivity to amphetamine. Collectively, these results confirm that high-quality aptamers for (+)-methamphetamine can indeed be isolated with an appropriately designed SELEX trial.

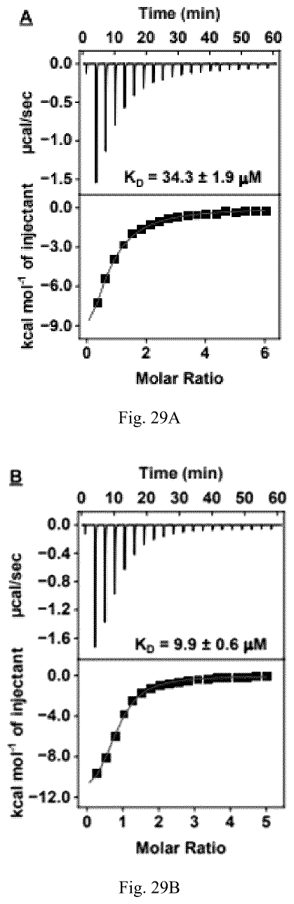

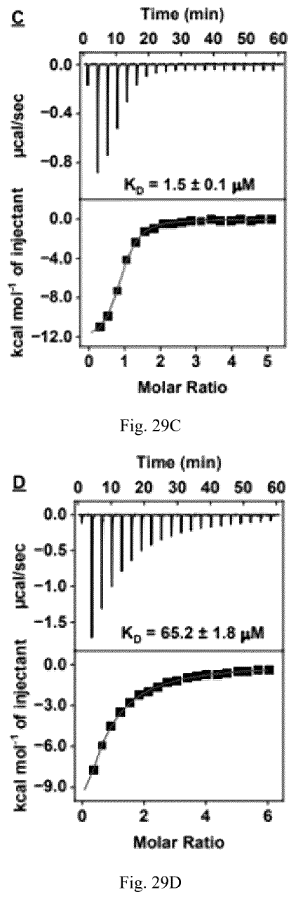

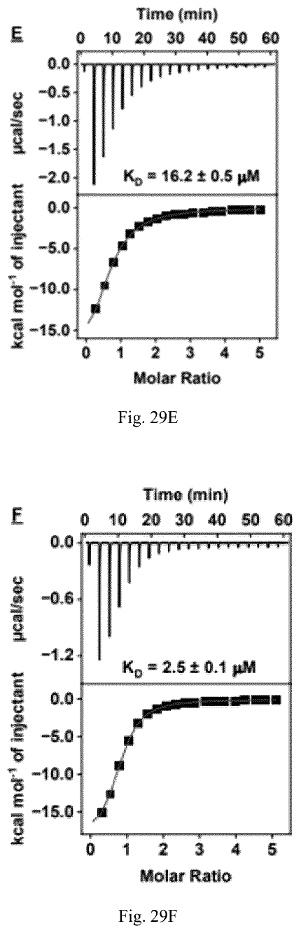

Since a relatively high concentration of Mg²⁺ was employed for selection, the importance of the concentration of this divalent cation on the binding affinity of the isolated aptamers was next determined. To do so, ITC with MU and ML4 for (+)-methamphetamine in 0.5× PBS (pH 7.4) plus 1, 2, or 5 mM MgCl₂ was performed. In general, the affinity of both aptamers increased as the concentration of Mg²⁺ increased. Specifically, the K_{D} of ML3 for (+)-methamphetamine was 34.3 M, 9.9 M, and 1.5 μM in 0.5× PBS buffer containing 1, 2, or 5 mM MgCl₂ (FIGS. 29A-29C), respectively; for ML4, K_{D}s were respectively 65.2 M, 16.2 M, and 2.5 μM (FIGS. 29D-29F). In comparison to the work by Carothers et al., the authors observed that the largest decrease in affinity (measured as ΔΔG) when Mg²⁺ was decreased from 5 mM to 1 mM was ˜1,000 cal/mol. In contrast, aptamers ML3 and MLM experienced a much larger decrease in affinity, with a ΔΔG of ˜1,800 cal/mol and 1,920 cal/mol, respectively. These results therefore indicate that ML3 and ML4 require Mg²⁺ to bind methamphetamine, and it is possible that this ion stabilizes a critical binding-competent conformation of the aptamers.

### High-Specificity Binding by (+)-Methamphetamine Aptamers and Comparison to Antibodies

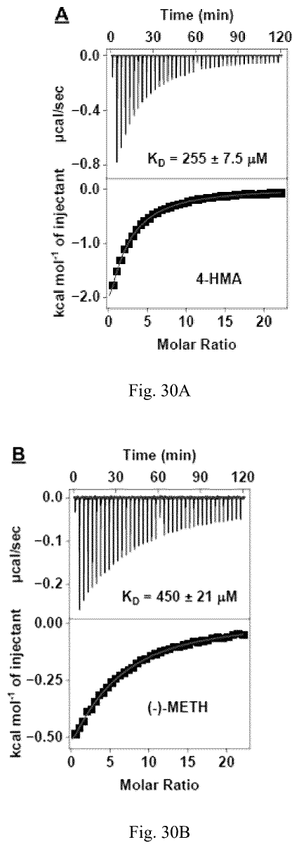

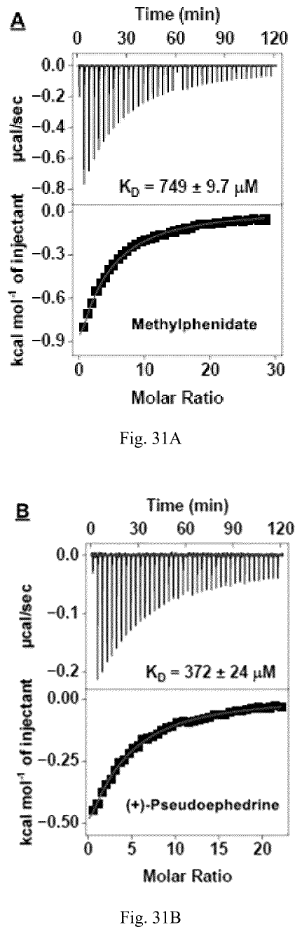

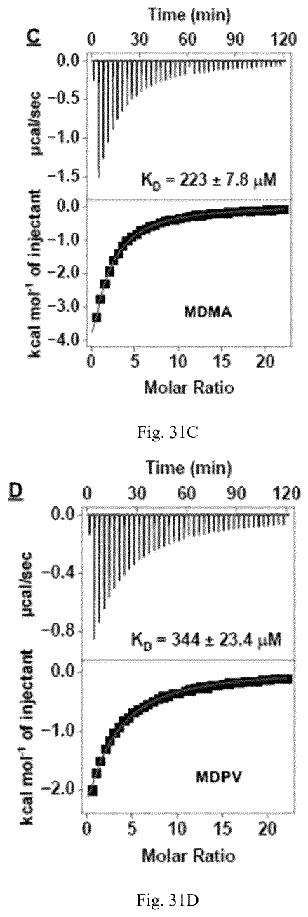

For sensing applications, receptors for (+)-methamphetamine must be able to reject structurally similar interferent molecules. It is well-documented in the literature that immunoassays for (+)-methamphetamine often cross-react to analogs like amphetamine, bupropion, and MDMA. In stark contrast, aptamer ML4 rejects these structurally similar molecules, which indicates that aptamers have superior specificity over antibodies for this target. The capability of aptamers like ML4 to discriminate between (+)-methamphetamine and amphetamine is quite impressive, as they only differ by a single methyl group. Similar discrimination has been seen with a previously published theophylline aptamer that favors theophylline binding 10,000-fold relative to caffeine, which also differs by a single methyl group. However, recognizing (+)-methamphetamine but not amphetamine is arguably a more challenging feat, because the methyl group of (+)-methamphetamine leaves its amino group with one less hydrogen bond, which one would expect to result in weaker affinity. Despite this, it is observed that ML4 prefers (+)-methamphetamine relative to amphetamine, by 50-fold (FIG. 6G). While it has been documented that some antibodies can distinguish between (+)-methamphetamine and amphetamine, these antibodies are unable to discriminate between (+)-methamphetamine and MDMA and other analogs. To determine whether aptamers could achieve this level of molecular discrimination, the affinity of MLM to a variety of structurally similar interferents was quantified using ITC. Impressively, it was found that MLM has 180-, 100-, 150-, 90-, 300-, and 140-fold lower affinity for (−)-methamphetamine (K_{D}=450±21 μM), 4-HMA (K_{D}=255±8 μM), (+)-pseudoephedrine (K_{D}=372±24 μM), MDMA (K_{D}=223±8 μM), methylphenidate (K_{D}=749±10 μM), and MDPV (K_{D}=344±23 μM), respectively, relative to (+)-methamphetamine (FIGS. 30A-30B and FIGS. 31A-31D). Based on three-dimensional structures of reported high-affinity riboswitches, it was hypothesized that this improved specificity from aptamers may arise because they have greater flexibility and can completely envelope their small molecule target, thus enforcing strict requirements for guest size, shape, and electrostatics.

### Analysis of the SELEX Trials and Comparison of (+)-Methamphetamine Aptamers

Through the four independent SELEX experiments performed, the dramatic influence that different selection parameters have on the outcome of SELEX has been demonstrated. In the first trial using an N30 library, where counter-SELEX was performed with numerous structurally similar compounds, no aptamers were identified. When counter-SELEX was skipped in the second trial, several (+)-methamphetamine aptamers with an average K_{D} of 8.9±6.9 μM for (+)-methamphetamine were discovered, but these aptamers cross-reacted to more than a dozen other molecules, such as amphetamine, MDMA, 4-HMA, 4-HA, and lidocaine. This suggests that there were most likely no highly specific sequences in the N30 library that could bind (+)-methamphetamine while also rejecting structurally similar interferent molecules. In the third trial, an N40 library was employed, and the counter-SELEX process was restored, and a notable improvement in aptamer affinity and specificity was observed. Aptamers with an average K_{D} of 6.6±2.3 μM for (+)-methamphetamine, with specificities that were likewise improved relative to the previous trial were obtained. Indeed, the best aptamer only cross-reacted to five of the nontarget molecules. In the final trial, buffer ionic conditions were adjusted and the counter-SELEX process with an N40 library isolated higher affinity and specific aptamers with an average K_{D} of 3.6±2.4 μM. For the best aptamer, near-perfect specificity was observed, with only minor cross-reactivity (<20%) to 4-HMA. These findings suggest that the recognition of low-complexity small molecules such as (+)-methamphetamine may require relatively larger aptamers to achieve high-affinity, highly specific recognition.

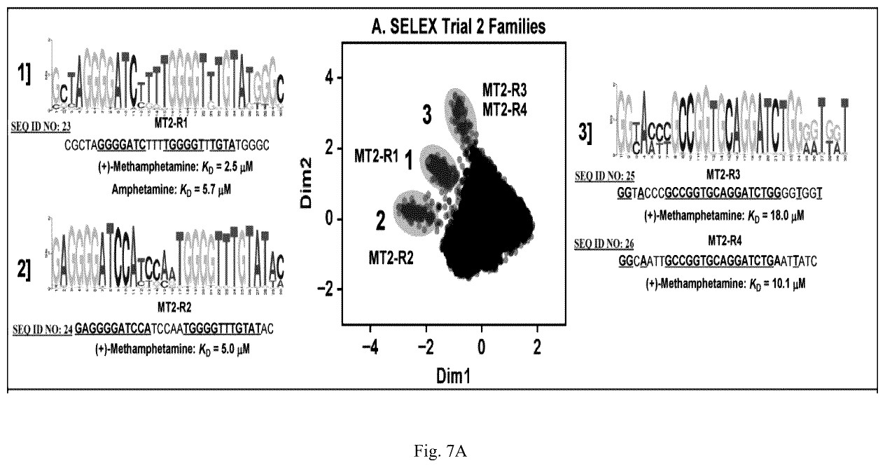

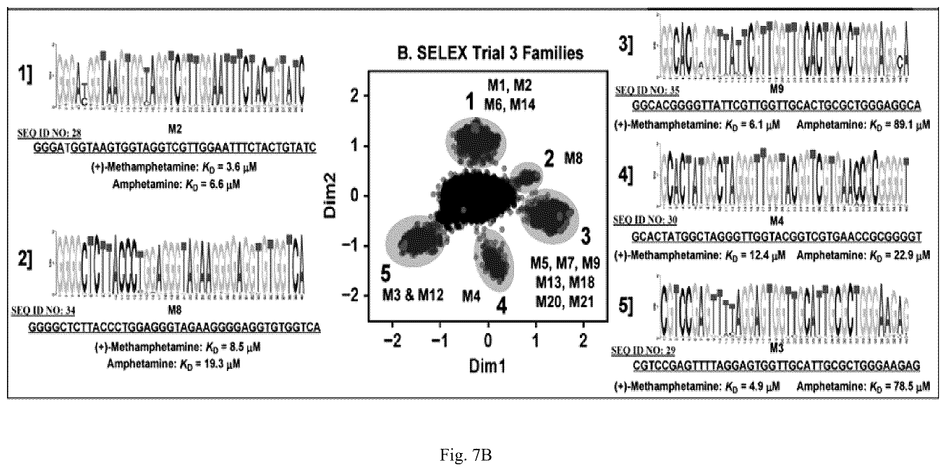

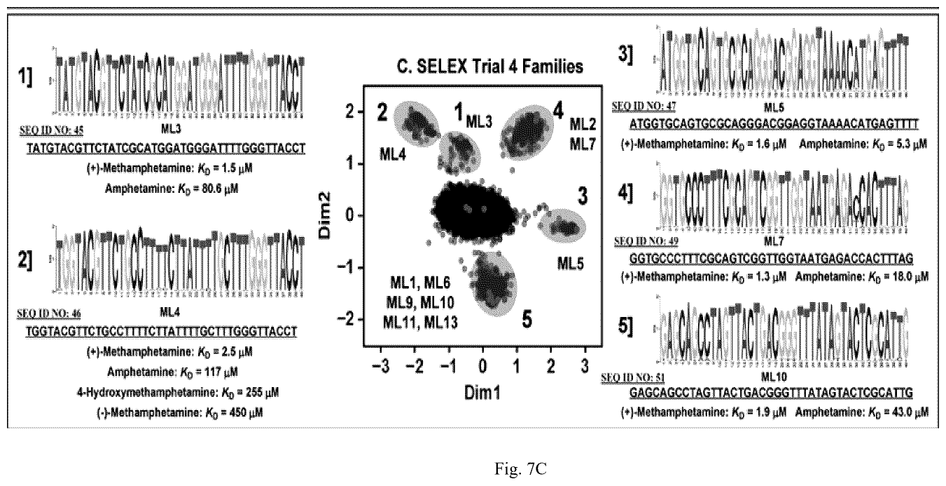

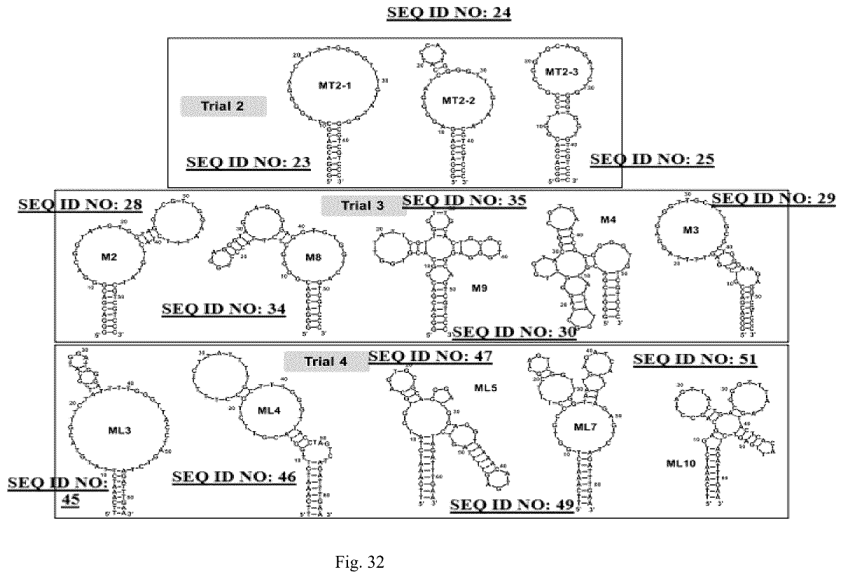

Analysis of the HTS data from the different trials provides some clues for the basis of the differing binding properties of the resulting (+)-methamphetamine aptamers. The bioinformatics software Raptgen was used to identify families in each trial based on their sequence similarity. Raptgen uses variational autoencoders to map HTS data onto a low-dimensional latent space, enabling the convenient identification of families with conserved motifs based on the formation of clusters in two-dimensional plots. To gain structural insights, NUPACK (46) was then used to predict the secondary structures of a few aptamers from each family (FIG. 32). For the second SELEX trial, three different families of sequences were identified (FIG. 7A). The first family contained a 17-nt consensus sequence flanked by regions of low consensus. The other two families were similar in sequence, containing two high-consensus GGGG repeats linked by four or five nucleotides of low consensus. Only 50-60% of the 30 nt binding domain was highly consensus-prone, indicating that only a portion of the binding domain is crucial for target recognition. Therefore, these families were characterized as having low complexity and unsurprisingly, these aptamers also had the poorest affinity and specificity among all the aptamers identified. This relationship between binding performance and sequence complexity mirrors previous studies showing that sequences with low information content have inferior binding properties relative to those that are more information rich (Carothers et al. 2004). In contrast, when Raptgen was applied to the final-round SELEX pool from the third trial, five different families were identified, all of which had high-consensus regions spanning nearly the entire binding domain (˜90%) (FIG. 7B). As expected, these sequences also had better affinity and specificity than aptamers from N30 libraries, further supporting the notion that more information-rich libraries with larger randomized domains yield aptamer candidates with superior overall binding properties. When examining the final SELEX pool from the fourth trial, five families that all had very high consensus (>95% conservation) for all 40 nt were observed (FIG. 7C). These aptamers had the best affinity and specificity, with some aptamers (e.g., MU and ML4) capable of discriminating amphetamine and (−)-methamphetamine from (+)-methamphetamine with a greater than or equal to 50-fold affinity difference. This provides strong evidence that the more nucleobases that are involved in target recognition, the better the binding performance of the aptamer. Notably, the best performing aptamer, ML4, was a member of a family that was unusually T-rich: >50% of the nucleobases were Ts. However, there were no more than three As in the sequences from this family, implying that these Ts were not participating in canonical A-T Watson-Crick pairing but were instead contributing to a noncanonical structure or directly involved in target recognition. Other well-performing aptamers were members of another distinct family that was relatively T-rich (40%), but with greater representation of A and G bases. This indicates that there are multiple different oligonucleotide architectures capable of (+)-methamphetamine recognition. Finally, no overlap in sequences between any of the SELEX trials was observed, which is unsurprising since N40 libraries have a theoretical sequence space of 1024 well beyond the initial sampling of 1014 library sequences.

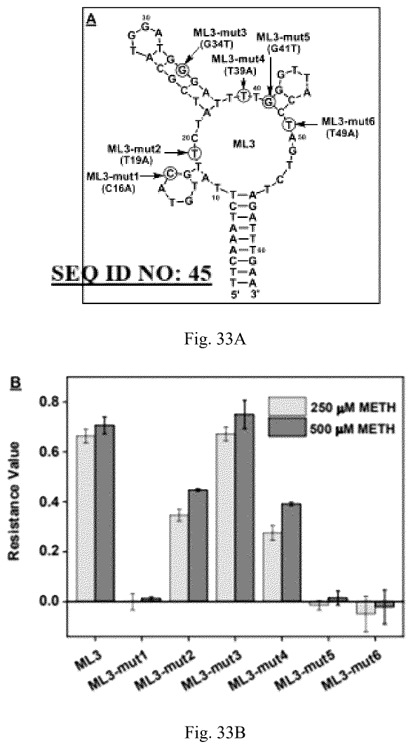

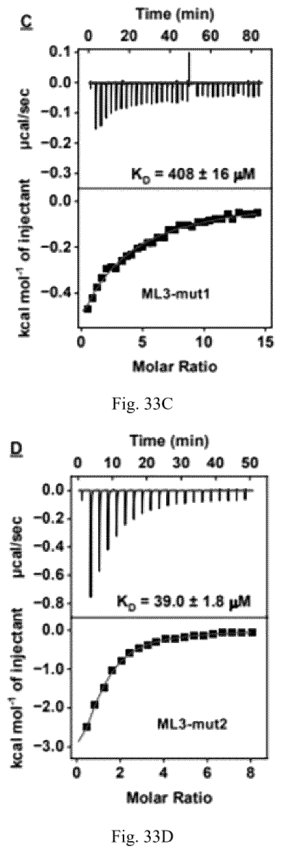

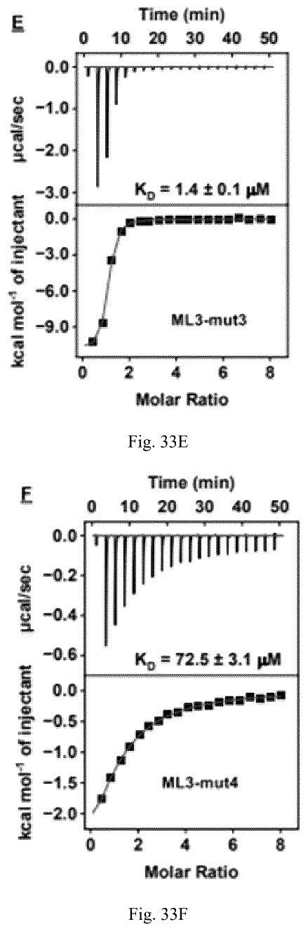

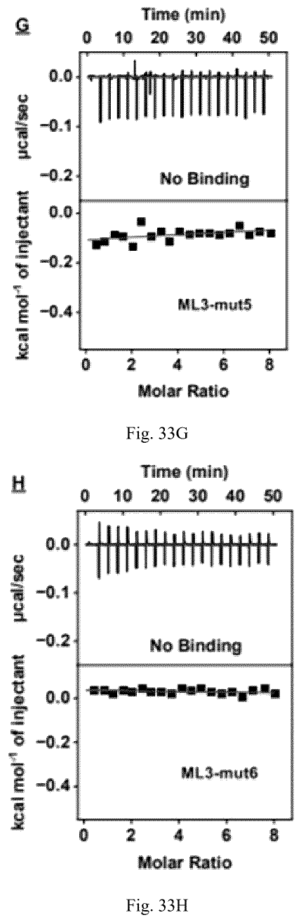

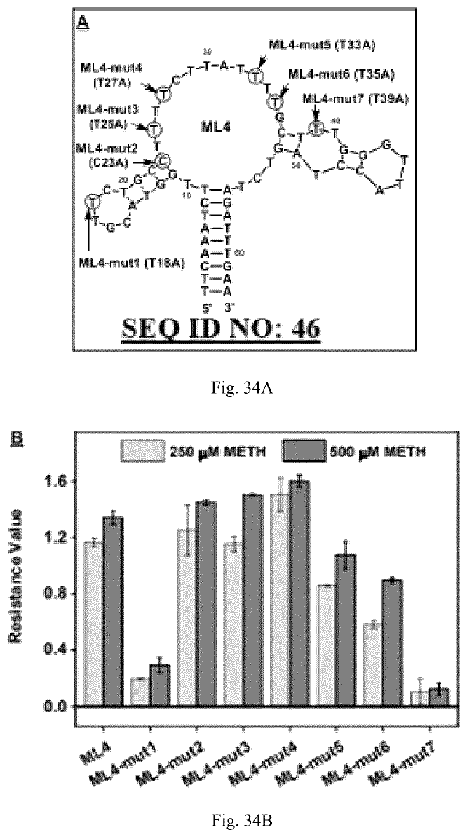

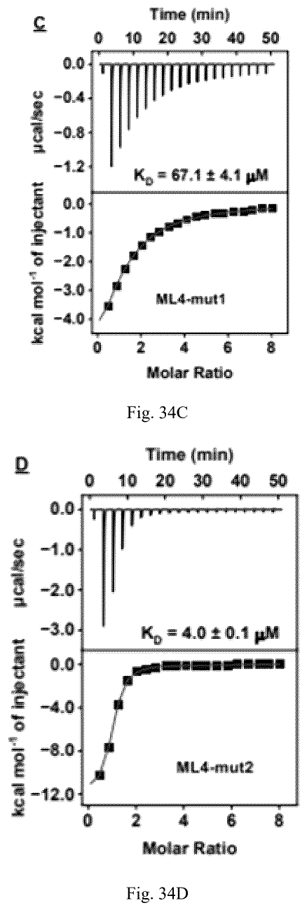

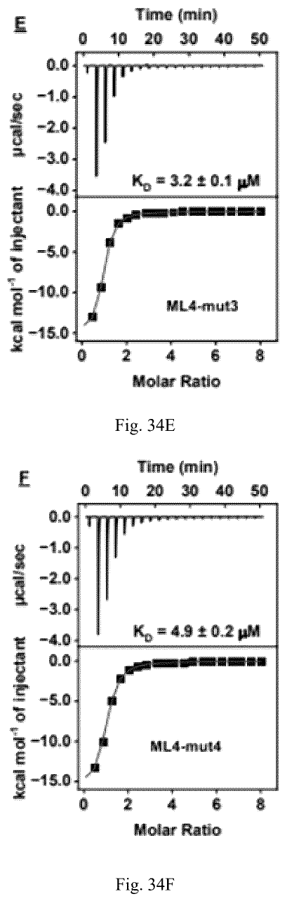

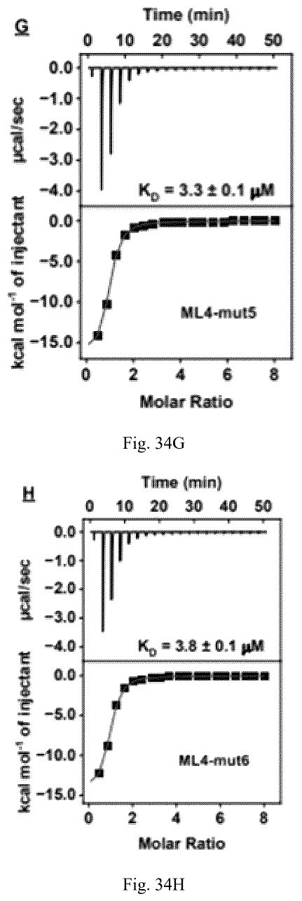

To more clearly define the contribution of various nucleobases in ML3 and ML4 for target recognition, various point mutants of ML3 and ML4 were designed and their affinity for (+)-methamphetamine was determined using the exonuclease digestion assay and ITC. For ML3, six different mutants were created by either changing C16 to A (SEQ ID NO:55), T19 to A (SEQ ID NO: 56), G34 to T (SEQ ID NO: 57), T39 to A (SEQ ID NO: 58), G41 to T (SEQ ID NO: 59), or T49 to A (SEQ ID NO: 60), which were termed ML3-mut1, -mut2, -mut3, -mut4, -mut5, and -mut6, respectively (FIG. 33A and Table 10). The exonuclease assay indicated that ML3-mut1, -mut5, and -mut6 had little or no affinity for methamphetamine, while ML3-mut2 and -mut4 had weaker affinity relative to ML3, and ML3-mut3 retained similar affinity to ML3 (FIG. 33B). The ITC data corroborated well with the exonuclease assay, with K_{D}s for (+)-methamphetamine of 408±16 μM, 39±2 μM, 1.4±0.1 M, 73±3 μM, >1 mM, and >1 mM for ML3-mut1, -mut2, -mut3, -mut4, -mut5, and -mut6, respectively (FIGS. 33C-33H and Table 8). These data indicate that C16, G41, and T49 are essential for methamphetamine binding. For MLM, the role of the thymine bases in this pyrimidine-rich aptamer was of particular interest. Therefore, seven different point mutants were created by respectively changing T18 (SEQ ID NO: 61), C23 (SEQ ID NO: 62), T25 (SEQ ID NO: 63), T27 (SEQ ID NO: 64), T33 (SEQ ID NO: 65), T35 (SEQ ID NO: 66), and T39 (SEQ ID NO: 67) to A (termed ML4-mut1, -mut2, -mut3, -mut4, -mut5, -mut6, and -mut7, respectively) (FIG. 34A and Table 10). The exonuclease assay indicated that only ML4-mut1 and -mut7 had heavily impaired affinity relative to ML4, with ML4-mut5 and -mut6 only having slightly lower affinity (FIG. 34B). Again, ITC data corroborated well with these data from the enzyme assay, with K_{D}s of 67±4 μM, 4±0.1 M, 3.2±0.1 M, 4.9±0.2 μM, 3.3±0.1 μM, 3.8±0.1 M, 64±4 μM for (+)-methamphetamine (FIGS. 34C-34I and Table 8), indicating that T18 and T39 are important contributors to target binding.

Detection of (+)-Methamphetamine in Oral Fluid with an Aptamer-Based Dye Displacement Assay

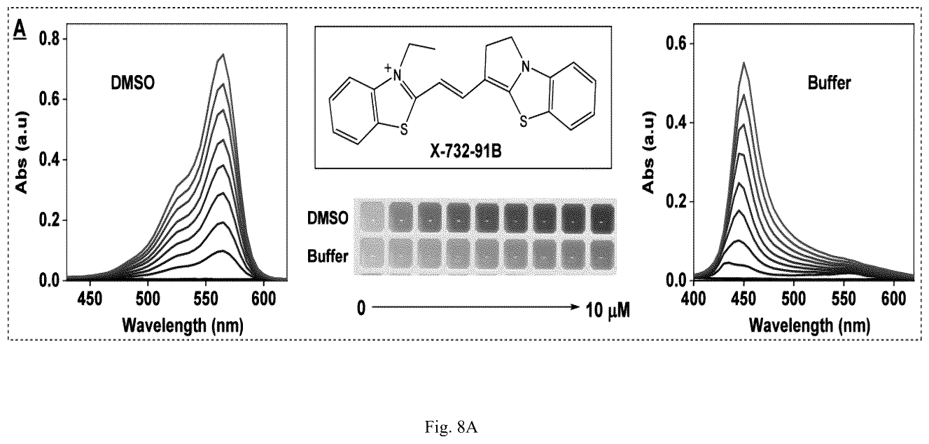

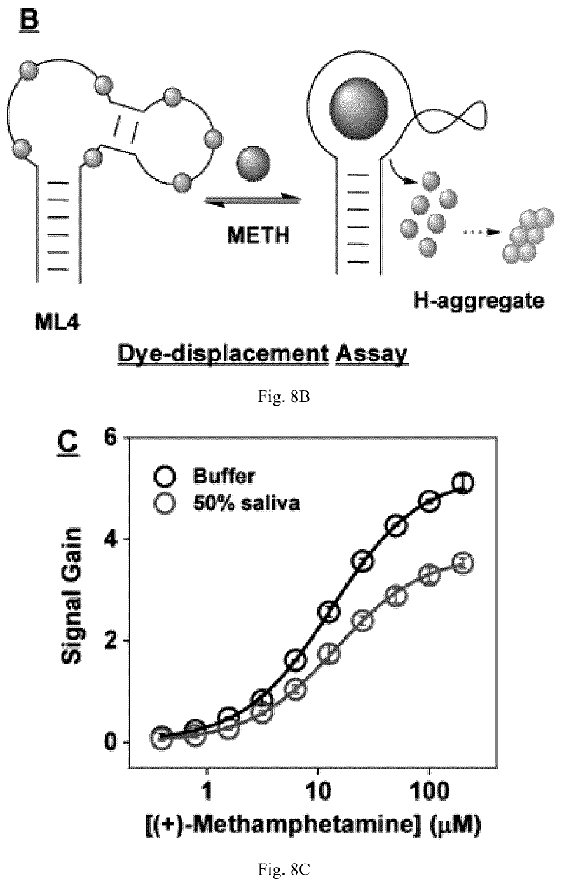

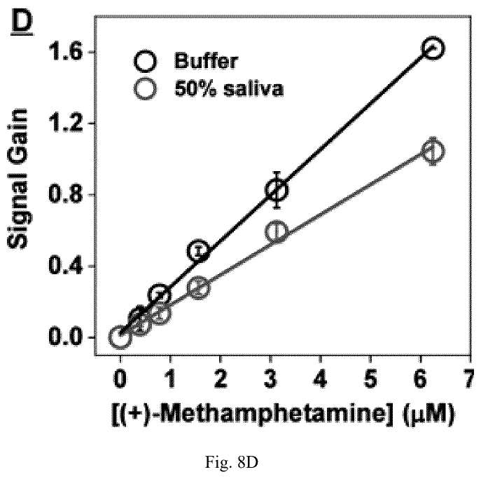

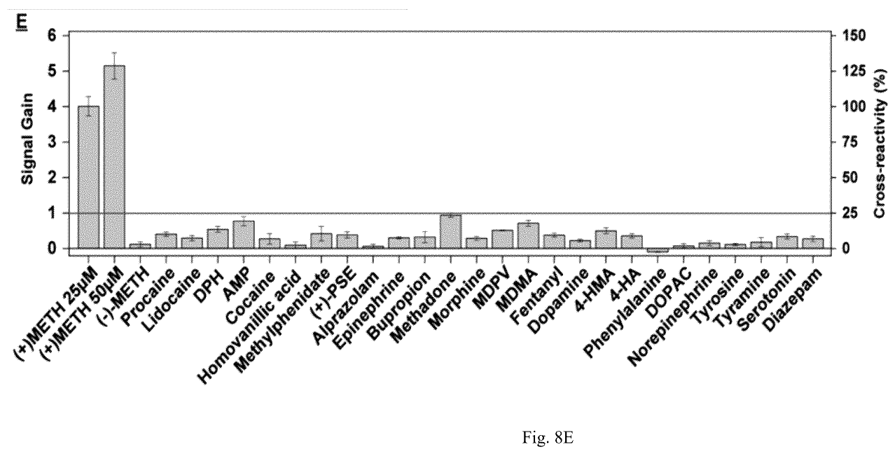

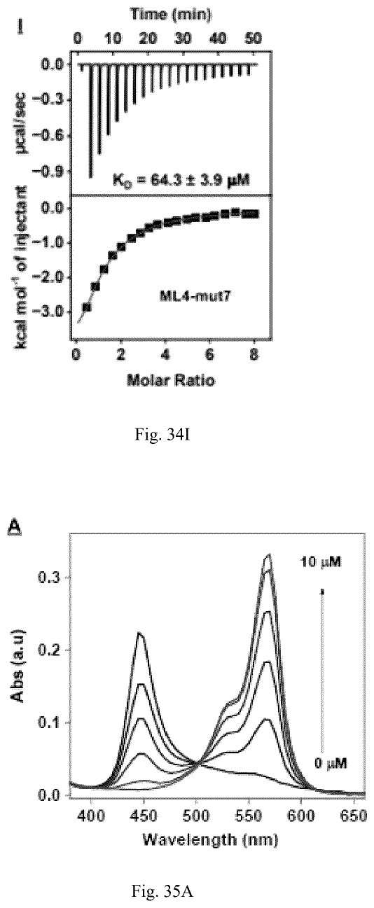

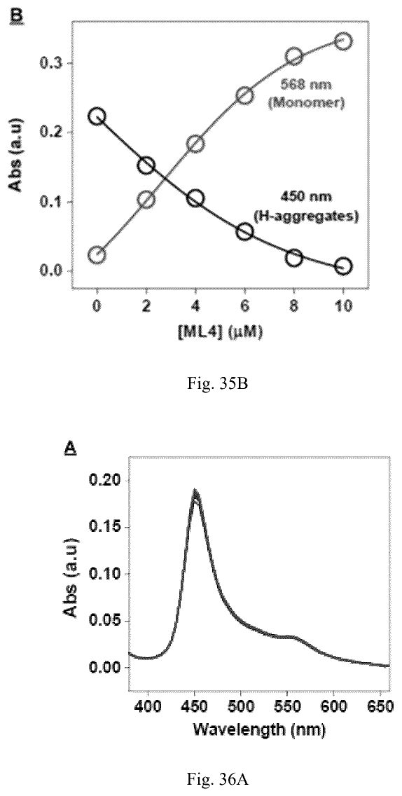

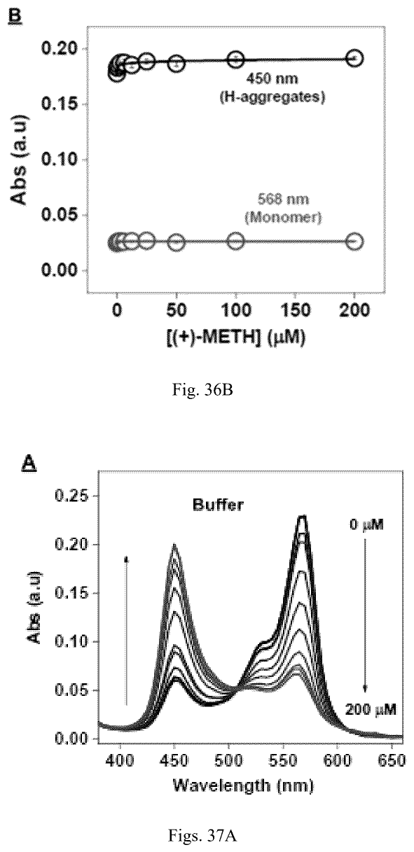

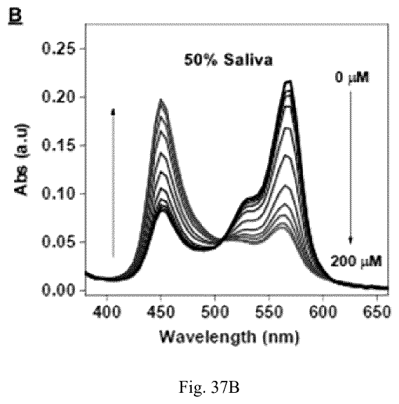

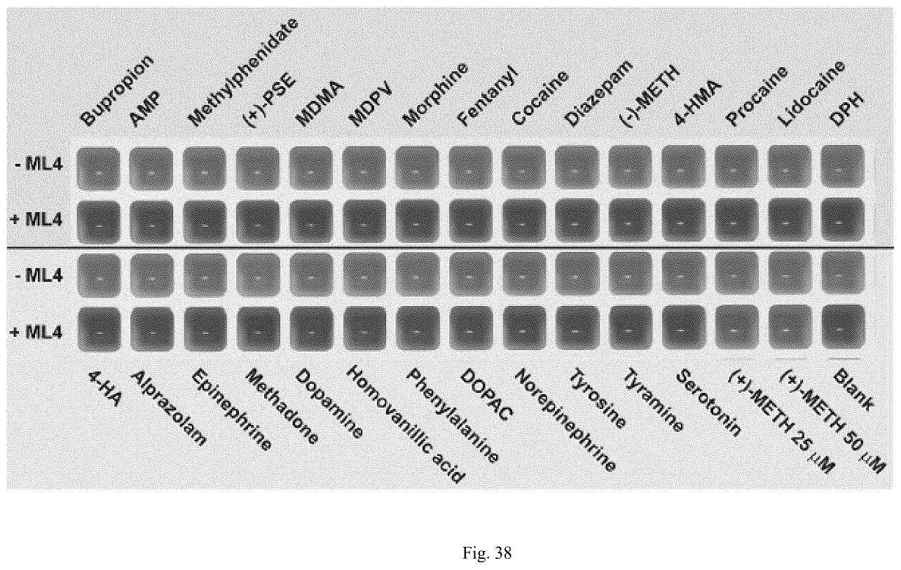

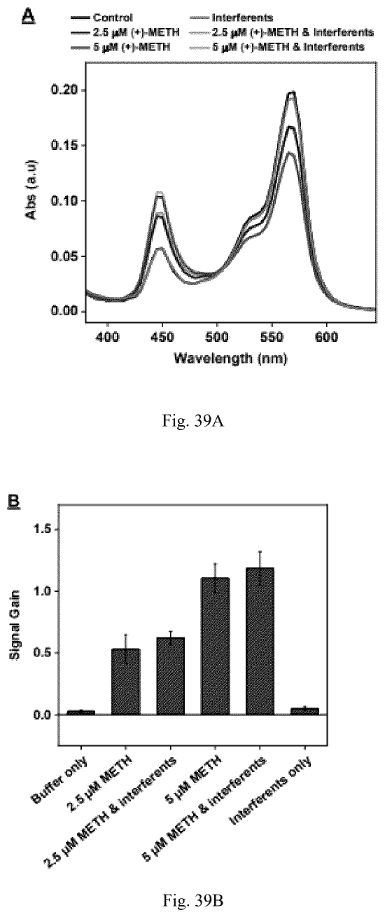

Finally, it was demonstrated that the aptamers are capable of rapid and facile detection of (+)-methamphetamine in oral fluid. A dye-displacement assay was developed based on aptamer ML4 and the cyanine dye X-732-91B. This dye maximally absorbs at 568 nm as a monomer in DMSO, producing a hot-pink color (FIG. 8A), but forms H-aggregates with an absorbance maximum at 450 nm in aqueous solution, yielding a yellow color. When the dye is titrated with increasing concentrations of aptamer in aqueous buffer, the absorbance peak at 450 nm diminishes while the peak at 568 nm grows, indicating the conversion of free aggregates to aptamer-bound monomers (FIGS. 35A-35B). When (+)-methamphetamine was titrated into a mixture of 6 μM aptamer and 4 μM dye, the target displaced the dye from the aptamer-dye complexes and these released dye molecules formed H-aggregates in solution (FIG. 8B). This resulted in a nearly immediate decrease in monomer absorbance and an increase in H-aggregate absorbance. The concentration of (+)-methamphetamine was quantified based on the ratio of aggregate:monomer absorbance, obtaining an instrumental LOD of 390 nM and a linear range of 0-6.25 μM (FIGS. 8C-8D). In a control experiment, it was confirmed that (+)-methamphetamine did not affect the absorbance spectrum of the dye itself (FIGS. 36A and 36B). Notably, this assay worked equally well in both buffer and 50% saliva (FIGS. 8C and 8D and FIGS. 37A and 37B) with identical detection limits. The response of the assay to a panel of structurally similar compounds and common drugs of abuse was also determined and no meaningful cross-reactivity was observed (FIG. 8E and FIG. 38). It was finally demonstrated that the assay can specifically detect (+)-methamphetamine even when it is present in a mixture of drugs. Specifically, it was observed that the response of the assay to 2.5 μM or 5 μM (+)-methamphetamine was similar whether the target was alone or in a mixture of 1 μM morphine, 3 μM cocaine, 2 μM methadone, and 1 μM fentanyl. Additionally, the assay did not respond to the drug mixture itself (FIGS. 39A and 39B). These results thus demonstrate that this assay is highly specific for (+)-methamphetamine.

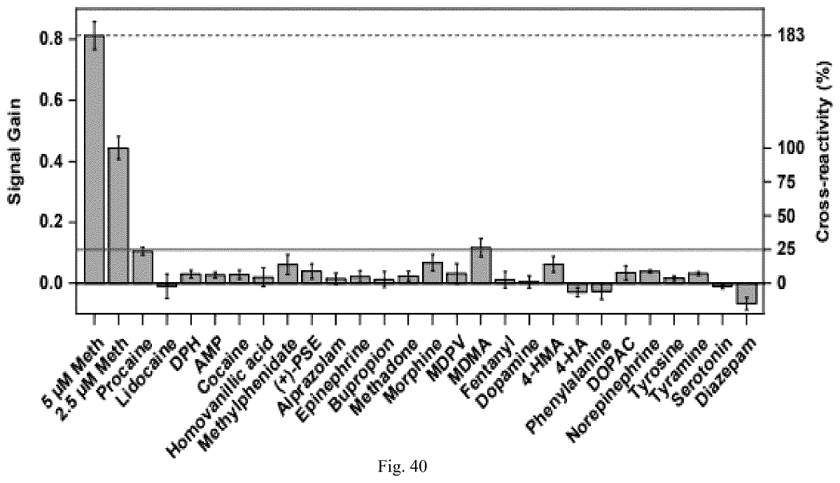

The concentration of methamphetamine in human saliva can reach ˜1-3 μM between 2-4 h after consumption and declines slowly thereafter, reaching ˜0.5-0.8 μM at 8 h post consumption, and ˜0.1 μM by 24 h. (48-50) Accordingly, it was hypothesized that the colorimetric assay can be used to determine recent (+)-methamphetamine use in oral fluid. To demonstrate this, the response of the assay to clinically relevant methamphetamine concentrations and interferent concentrations that are, in some cases, 100-fold higher than maximum clinically relevant levels were determined. A clearly detectable signal at this target concentration was observed, with <20% cross-reactivity to almost all interferents; 26% cross-reactivity to a 6-fold higher concentration of MDMA (15 μM) relative to 2.5 μM (+)-methamphetamine, and 24% cross-reactivity to procaine at a 16-fold higher concentration (40 μM) was observed (FIG. 40). Thus, the designed assay is specific enough to detect methamphetamine in saliva for clinical/toxicological purposes.

### DISCUSSION

The current application systematically investigates the difficulties associated with isolating high-performance aptamers for the small-molecule target (+)-methamphetamine via in vitro selection and identified strategies to successfully isolate aptamers for such targets. First it was confirmed that previously reported aptamers for methamphetamine have either low or no affinity for this target, and it was noted that this is due to the unsuitability of the selection strategies employed to isolate these aptamers. These include target-immobilized SELEX, which masks functional groups on targets and hence prevents aptamers from fully interacting with the target, and graphene-oxide SELEX, which has an order of magnitude lower separation efficiency than library-immobilized SELEX based on recent findings from the Liu group. These aptamers were tested using ITC. Since in some cases ligand binding does not release a meaningful amount of heat, ITC will not be able to determine binding affinity. There are alternative gold standard affinity determination methods such as surface plasmon resonance, biolayer interferometry, and microscale thermophoresis. However, the aptamer-methamphetamine binding may be too subtle to produce a detectable signal in these platforms (i.e., a change in surface refractive index, biolayer thickness, or thermophoretic mobility) due to the low molecular weight of the target (149 Da). For this reason, an exonuclease digestion assay, a well-established approach for determining aptamer-ligand binding properties, was utilized to support findings, which were found to be concordant with ITC results.

Next it is demonstrated that the selection of high-performance aptamers for low-epitope targets such as methamphetamine is challenging. After performing four SELEX trials, several strategies were identified to facilitate isolation of aptamers for this target. In the first trial of SELEX using an N30 library with performance of stringent counter-SELEX, no aptamers for (+)-methamphetamine were enriched. This was most likely because there were no highly specific (+)-methamphetamine aptamers in the N30 library, and performing counter-SELEX against amphetamine and MDMA removed all of these (+)-methamphetamine aptamers from the pools. In the second trial, SELEX was performed without counter-SELEX to determine if aptamers could be isolated for (+)-methamphetamine, and results indicated found that it was indeed possible. Thus, when SELEX is initially performed for a low-complexity target, it may be preferable to withhold counter-SELEX to determine whether aptamers for that target can be enriched and how specific those aptamers could be. If these aptamers exhibit poor specificity, this information can be used to determine which molecules to include for counter-SELEX in the next trial. It was also found that the discovery of highly specific aptamers for a low-complexity molecule like (+)-methamphetamine requires libraries with longer random regions, given the success with the N40 libraries used in the third and fourth trials relative to the failures with N30 libraries in the first two trials. This is in agreement with increasing evidence from the literature that high-quality aptamers for small molecule analytes with few epitopes can only be discovered with more complex random libraries. Additionally, if a selection fails, one should consider altering the ionic strength of the buffer; in particular, adjusting the concentration of Mg²⁺ can increase the likelihood of successfully identifying aptamers by influencing the conformation and hence the function of nucleic acids. Finally, the inclusion of surfactants like Triton X-100 in the selection buffer increased the efficiency with which binding-incompetent sequences are eliminated during library-immobilized SELEX. If there are concerns about the influence of surfactants on target dissolution or stability, these surfactants can be excluded from the buffer when the library is challenged with the target.

### Example 2: Development of Electrochemical Aptamer-Based (EAB) Sensors for (+)-Methamphetamine Detection

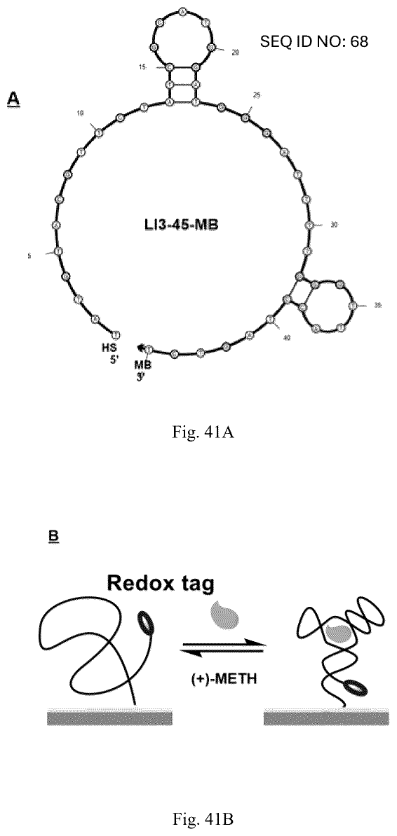

To allow for rapid, selective, and sensitive detection of (+)-methamphetamine, a modified structure switching version of LI3 (LI3-45; SEQ ID NO: 68) (FIG. 41A) was utilized to fabricate electrochemical aptamer-based (EAB) sensors. The signaling mechanism of EAB sensors is based on a target-binding-induced conformation change of methylene blue-modified aptamers immobilized onto gold electrodes, thereby altering the electron transfer efficiency between methylene blue and the electrode surface in the presence of target, producing a measurable signal which is used to quantify target concentration. To fabricate EAB sensors for (+)-methamphetamine detection, LI3-45-MB was synthesized with a 5′ thiol-C6 group and a 3′ methylene blue tag (FIG. 41B) and was immobilized onto the surface of cleaned gold electrodes through thiol-gold chemistry. Subsequent electrode passivation using a backfilling agent was used to remove nonspecifically adsorbed aptamers and prevent nonspecific adsorption of interferents in the sample matrix. Target response was reported as signal gain, where the difference between the measured current in the presence and absence of (+)-methamphetamine was divided by the measured current in the absence of (+)-methamphetamine and converted to a percentage.

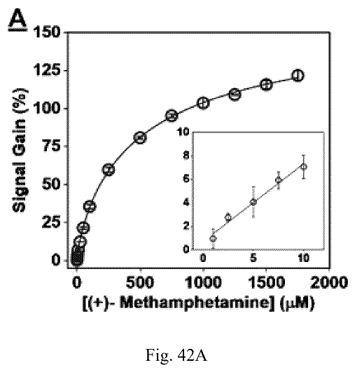

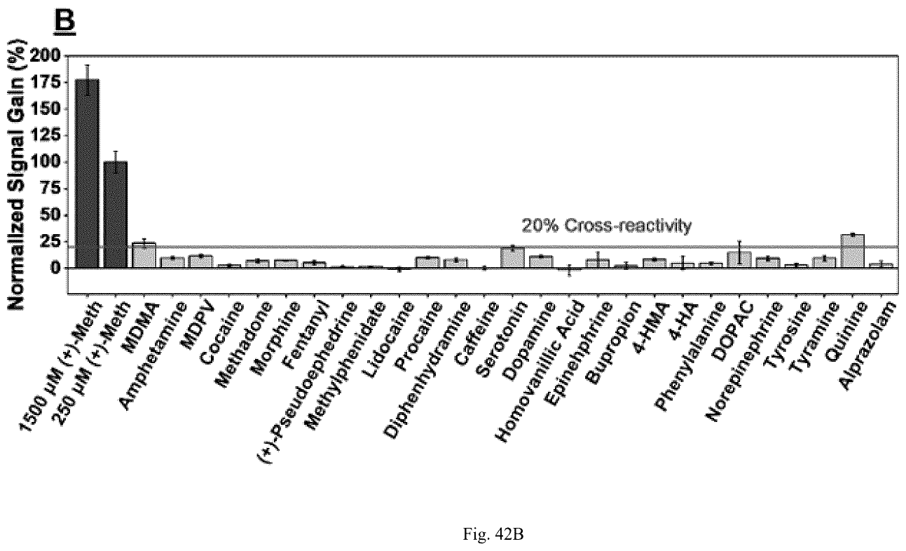

(+)-methamphetamine detection was performed using EAB sensor fabricated with LI3-45-MB. A concentration of LI3-45-MB at 400 nM and a frequency of 600 Hz resulted in a maximal signal gain of ˜86% in the presence of 500 μM (+)-methamphetamine. Calibration curves were performed in buffer (1× PBS, 2 mM MgCl₂, 0.005% v/v Triton X-100), where a detection limit of 1 μM (+)-methamphetamine and a linear range of 1-10 μM was observed (FIG. 42A). Specificity testing using 26 interferents at a concentration of 250 μM resulted in four non-target molecules having a cross reactivity greater than 20% (FIG. 42B).

### Tables

TABLE 1
List of DNA sequences used for SELEX.

| Name | Sequence |
|---|---|
| Trial 1 & 2 Library | GGAGGCTCTCGGGACGAC(N30)GTCGTCCCGCCTTTAGGA |
| | TTTACAG (SEQ ID NO: 1) |
| Trial 3 Library | GGAGGCTCTCGGGACGAC(N40)GTCGTCCCGCCTTTAGGA |
| | TTTACAG (SEQ ID NO: 2) |
| Trial 4 Library | TCCATAGCTCCTTGTTCAAATCT(N40)AGTCTAGATTTGAA |
| | TTCTCGGATACAACGA (SEQ ID NO: 3) |
| Trial 1-3 FP | GGAGGCTCTCGGGACGAC (SEQ ID NO: 4) |
| Trial 1-3 RP-bio | /5Bio/-CTGTAAATCCTAAAGGCGGGACGAC (SEQ ID NO: 5) |
| Trial 1-3 cDNA15-bio | GTCGTCCCGAGAGCCATA-/3Bio/ (SEQ ID NO: 6) |
| Trial 1-3 FP-HTS | ACACTCTTTCCCTACACGACGCTCTTCCGATCT(N45)GGAG |
| | GCTCTCGGGACGAC (SEQ ID NO: 7) |
| Trial 1-3 RP-HTS | GACTGGAGTTCAGACGTGTGCTCTTCCGATCT(N43)CTGTA |
| | AATCCTAAAGGCGGGACGAC (SEQ ID NO: 8) |
| Trial 4 FP | TCCATAGCTCCTTGTTCAAATCT (SEQ ID NO: 9) |
| Trial 4 RP-bio | /5Bio/TCGTTGTATCCGAGAATTCAAATCT (SEQ ID NO: 10) |
| Trial 4 cDNA15-bio | /5Bio/CGAGAATTCAAATCT (SEQ ID NO: 11) |
| Trial 4 FP-HTS | ACACTCTTTCCCTACACGACGCTCTTCCGATCT(N44)TCCA |
| | TAGCTCCTTGTTCAAA (SEQ ID NO: 12) |
| Trial 4 RP-HTS | GACTGGAGTTCAGACGTGTGCTCTTCCGATCT(N45)TCGTT |
| | GTATCCGAGAATTCA (SEQ ID NO: 13) |

/5Bio/ = 5′ biotin modification; /3Bio/ = 3′ biotin modification.

TABLE 2
Selection strategy and conditions for the first trial of methamphetamine SELEX.

| | Pool | | | | |
|---|---|---|---|---|---|
| | Size | Buffer | | Buffer | [Target] |
| Round | (pmol) | Wash | Counter-targets | Wash | (μM) |
| 1 | 1,000 | 10 | None | None | 100 |
| 2 | 447 | 20 | 100 uM PRO (250 μL × 3), 100 μM LIDO | 28 | 100 |
| | | | (250 μL × 3), 100 μM CAF (250 μL × 3), | | |
| | | | 100 μM PMZ (250 μL × 3), 100 μM QUI | | |
| | | | (250 μL × 3), 100 μM DPH (250 μL × 3), | | |
| | | | 100 μM FEN (250 μL × 3), 100 μM COC | | |
| | | | (250 μL × 3) | | |
| 3 | 300 | 20 | 100 μM PRO (250 μL × 3), 100 μM LIDO | 30 | 100 |
| | | | (250 μL × 3), 100 μM CAF (250 μL × 3), | | |
| | | | 100 μM PMZ (250 μL × 3), 100 μM QUI | | |
| | | | (250 μL × 3), 100 μM DPH (250 μL × 3), | | |
| | | | 100 μM FEN (250 μL × 3), 100 μM COC | | |
| | | | (250 μL × 3), 100 μM AMP (250 μL × 3), | | |
| | | | 100 μM DOP (250 μL × 3), 100 μM 4-HMA | | |
| | | | (250 μL × 3), 100 μM TYM (250 μL × 3), | | |
| | | | 100 μM 4-HA (250 μL × 3), 100 μM | | |
| | | | SER (250 μL × 3) | | |
| 4 | 300 | 20 | 100 μM PRO (250 μL × 3), 100 μM LIDO | 30 | 100 |
| | | | (250 μL × 3), 100 μM CAF (250 μL × 3), | | |
| | | | 100 μM PMZ (250 μL × 3), 100 μM QUI | | |
| | | | (250 μL × 3), 100 μM DPH (250 μL × 3), | | |
| | | | 100 μM FEN (250 μL × 3), 100 μM COC | | |
| | | | (250 μL × 3), 100 μM AMP (250 μL × 3), | | |
| | | | 100 μM DOP (250 μL × 3), 100 μM 4-HMA | | |
| | | | (250 μL × 3), 100 μM TYM (250 μL × 3), | | |
| | | | 100 μM 4-HA (250 μL × 3), 100 μM SER | | |
| | | | (250 μL × 3), 100 μM DOPAC (250 μL × 3), | | |
| | | | 100 μM HVA (250 μL× 3) | | |
| 5 | 300 | 20 | 100 μM PRO (250 μL × 3), 100 μM LIDO | 30 | 100 |
| | | | (250 μL × 3), 100 μM PMZ (250 μL × 3), | | |
| | | | 100 μM CAF + QUI (250 μL × 3), 100 μM | | |
| | | | DPH (250 μL × 3), 100 μM FEN (250 | | |
| | | | μL × 6), 100 μM COC (250 μL × 3), | | |
| | | | 100 μM DOPAC + HVA (250 μL × 3), 100 μM | | |
| | | | PSU (250 μL × 3), 100 μM NE + EPI (250 | | |
| | | | μL × 3), 100 μM BPP (250 μL × 3), 100 μM | | |
| | | | METD + MOR (250 μL × 3), 100 μM | | |
| | | | MDPV + MDMA (250 μL × 3), 100 μM | | |
| | | | AMP (250 μL × 3),100 μM DOP (250 | | |
| | | | μL × 3), 100 μM 4-HMA (250 μL × 3), | | |
| | | | 100 μM TYM (250 μL × 3), 100 μM 4-HA | | |
| | | | (250 μL × 3), 100 μM SER (250 μL × 3) | | |
| 6 | 300 | 30 | 100 μM PRO (250 μL × 3), 100 μM LIDO | 30 | 75 |
| | | | (250 μL × 3), 100 μM PMZ (250 μL × 3), | | |
| | | | 100 μM CAF + QUI (250 μL × 3), 100 μM | | |
| | | | DPH (250 μL × 3), 100 μM FEN (250 | | |
| | | | μL × 6), 100 μM COC (250 μL × 3), | | |
| | | | 100 μM DOPAC + HVA (250 μL × 3), 100 μM | | |
| | | | MPD (250 μL × 3), 100 μM PSU (250 | | |
| | | | μL × 3), 100 μM NE + EPI (250 μL × 3), | | |
| | | | 100 μM BPP (250 μL × 3), 100 μM METD + | | |
| | | | MOR (250 μL × 3), 100 μM MDPV + | | |
| | | | MDMA (250 μL × 3), 100 μM AMP (250 | | |
| | | | μL × 3), 100 μM DOP (250 μL × 3), 100 μM | | |
| | | | 4-HMA (250 μL × 3), 100 μM TYM (250 | | |
| | | | μL × 3), 100 μM 4-HA (250 μL × 3), 100 | | |
| | | | μM SER (250 μL × 3) | | |
| 7 | 300 | 30 | 250 μM PRO (250 μL × 3), 250 μM LIDO | 30 | 50 |
| | | | (250 μL × 3), 250 μM PMZ (250 μL × 3), | | |
| | | | 250 μM CAF + QUI(250 μL × 3), 250 μM | | |
| | | | DPH (250 μL × 3), 250 μM FEN (250 μL × | | |
| | | | 6), 250 μM COC (250 μL × 3), 250 μM | | |
| | | | DOPAC + HVA (250 μL × 3), 250 μM | | |
| | | | MPD (250 μL × 3), 50 μM ALP (250 μl × | | |
| | | | 3), 250 μM PSU (250 μL × 3), 250 μM | | |
| | | | NE + EPI (250 μL × 3), 250 μM BPP (250 | | |
| | | | μL × 3), 250 μM METD + MOR (250 μL × | | |
| | | | 3), 250 μM MDPV + MDMA (250 μL × | | |
| | | | 3), 250 μM AMP (250 μL × 3), 250 μM | | |
| | | | DOP (250 μL × 3), 250 μM 4-HMA (250 | | |
| | | | μL × 3), 250 μM TYM (250 μL × 3), 250 | | |
| | | | μM 4-HA (250 μL × 3), 250 μM SER (250 | | |
| | | | μL × 3), 250 μM TYR + PHE (250 μL × 3) | | |
| 8 | 300 | 30 | 250 μM PRO (250 μL × 3), 250 μM LIDO | 30 | 50 |
| | | | (250 μL × 3), 250 μM PMZ (250 μL × 3), | | |
| | | | 250 μM CAF + QUI(250 μL × 3), 250 μM | | |
| | | | DPH (250 μL × 3), 250 μM FEN (250 μL × | | |
| | | | 6), 250 μM COC (250 μL × 3), 250 μM | | |
| | | | DOPAC + HVA (250 μL × 3), 250 μM | | |
| | | | MPD (250 μL × 3), 50 μM ALP (250 μl × | | |
| | | | 3), 250 μM PSU (250 μL × 3), 250 μM | | |
| | | | NE + EPI (250 μL × 3), 250 μM BPP (250 | | |
| | | | μL × 6), 250 μM METD + MOR (250 μL × | | |
| | | | 3), 250 μM MDPV + MDMA (250 μL × | | |
| | | | 3), 250 μM AMP (250 μL × 3), 250 μM | | |
| | | | DOP (250 μL × 3), 250 μM 4-HMA (250 | | |
| | | | μL × 3), 250 μM TYM (250 μL × 3), 250 | | |
| | | | μM 4-HA (250 μL × 3), 250 μM SER (250 | | |
| | | | μL × 3), 250 μM TYR + PHE (250 μL × 3) | | |
| 9 | 300 | 30 | Same as previous round | 30 | 50 |
| 10 | 300 | 30 | Same as previous round | 30 | 50 |
| 11 | 300 | 30 | 250 μM PRO (250 μL × 3), 250 μM LIDO | 30 | 50 |
| | | | (250 μL × 3), 250 μM PMZ (250 μL × 3), | | |
| | | | 250 μM CAF + QUI(250 μL × 3), 250 μM | | |
| | | | DPH (250 μL × 3), 250 μM AMP (250 μL × | | |
| | | | 3), 250 μM COC (250 μL × 3), 250 μM | | |
| | | | DOPAC + HVA (250 μL × 3), 250 μM | | |
| | | | MPD (250 μL × 3), 50 μM ALP (250 μL × | | |
| | | | 3), 250 μM PSU (250 μL × 3), 250 μM | | |
| | | | NE + EPI (250 μL × 3), 250 μM BPP (250 | | |
| | | | μL × 6), 250 μM METD + MOR (250 μL × | | |
| | | | 3), 250 μM MDPV + MDMA (250 μL × | | |
| | | | 3), 250 μM FEN (250 μL × 6), 250 | | |

TABLE 3
Selection strategy and conditions for the
second trial of methamphetamine SELEX.

| | Pool Size | Buffer | Counter- | Buffer | [Target] |
|---|---|---|---|---|---|
| Round | (pmol) | Wash | targets | Wash | (μM) |
| 1 | 1,000 | 10 | None | None | 500 |
| 2 | 300 | 20 | None | None | 250 |
| 3 | 300 | 30 | None | None | 250 |
| 4 | 300 | 40 | None | None | 250 |
| 5 | 300 | 50 | None | None | 250 |
| 6 | 300 | 50 | None | None | 250 |
| 7 | 300 | 50 | None | None | 250 |
| 8 | 300 | 50 | None | None | 250 |
| 9 | 300 | 50 | None | None | 250 |
| 10 | 300 | 50 | None | None | 250 |
| 11 | 300 | 50 | None | None | 250 |

TABLE 4
Selection strategy and conditions for the third trial of methamphetamine SELEX.

| | Pool | | | | |
|---|---|---|---|---|---|
| | Size | Buffer | | Buffer | |
| Round | (pmol) | Wash | Counter-targets | Wash | [Target](μM) |
| 1 | 1,000 | 10 | None | None | 500 |
| 2 | 300 | 20 | 100 μM PRO (250 μL × 3), 100 μM LIDO | 20 | 250 |
| | | | (250 μL × 3), 100 μM CAF (250 μL × 3), | | |
| | | | 100 μM QUI (250 μL × 3), 100 μM DPH | | |
| | | | (250 μL × 3), 100 μM AMP (250 μL × 3) | | |
| 3 | 300 | 20 | 100 μM PRO (250 μL × 3), 100 μM LIDO | 30 | 250 |
| | | | (250 μL × 3), 100 μM CAF (250 μL × 3), | | |
| | | | 100 μM QUI (250 μL × 3), 100 μM DPH | | |
| | | | (250 μL × 3), 100 μM AMP (250 μL × 3), | | |
| | | | 100 μM DOP (250 μL × 3), 100 μM TYM | | |
| | | | (250 μL × 3), 100 μM 4-HA (250 μL × 3), | | |
| | | | 100 μM SER (250 μL × 3), 100 μM | | |
| | | | DOPAC (250 μL × 3), 100 μM HVA | | |
| | | | (250 μL × 3), 100 μM EPI (250 μL × 3) | | |
| 4 | 300 | 20 | 250 μM PRO (250 μL × 3), 250 μM LIDO | 30 | 100 |
| | | | (250 μL × 3), 250 μM CAF (250 μL × 3), | | |
| | | | 250 μM QUI (250 μL × 3), 250 μM DPH | | |
| | | | (250 μL × 3), 250 μM AMP (250 μL × 3), | | |
| | | | 100 μM DOP (250 μL × 3), 100 μM 4-HMA | | |
| | | | (250 μL × 3), 100 μM TYM (250 μL × 3), | | |
| | | | 100 μM 4-HA (250 μL × 3), 100 μM | | |
| | | | SER (250 μL × 3), 100 μM DOPAC (250 | | |
| | | | μL × 3), 100 μM HVA (250 μL × 3), 100 | | |
| | | | μM MPD (250 μL × 3), 100 μM ALP (250 | | |
| | | | μL × 3), 100 μM PSU (250 μL × 3), 100 | | |
| | | | μM NE (250 μL × 3), 100 μM EPI | | |
| | | | (250 μL × 3), 100 μM BPP (250 μL × 3) | | |
| 5 | 300 | 24 | 250 μM PRO (250 μL × 3), 250 μM LIDO | 30 | 100 |
| | | | (250 μL × 3), 250 μM CAF (250 μL × 3), | | |
| | | | 250 μM QUI (250 μL × 3), 250 μM DPH | | |
| | | | (250 μL × 3), 250 μM AMP (250 μL × 3), | | |
| | | | 250 μM DOP (250 μL × 3), 250 μM 4- | | |
| | | | HMA (250 μL × 3), 250 μM TYM (250 μL × | | |
| | | | 3), 250 μM 4-HA (250 μL × 3), 250 μM | | |
| | | | SER (250 μL × 3), 250 μM DOPAC (250 | | |
| | | | μL × 3), 250 μM HVA (250 μL × 3), 100 | | |
| | | | μM MPD (250 μL × 3), 100 μM ALP (250 | | |
| | | | μL × 3), 100 μM PSU (250 μL × 3), 100 | | |
| | | | μM NE (250 μL × 3), 100 μM EPI (250 μL × | | |
| | | | 3), 100 μM BPP (250 μL × 3), 100 μM | | |
| | | | METD (250 μL × 3), 100 μM MOR (250 | | |
| | | | μL × 3), 100 μM MDPV (250 μL × 3), 100 | | |
| | | | μM MDMA (250 μL × 3), 100 μM TYR + | | |
| | | | PHE (250 μL × 3) | | |
| 6 | 300 | 30 | 250 μM PRO (250 μL × 3), 250 μM LIDO | 30 | 100 |
| | | | (250 μL × 3), 250 μM CAF (250 μL × 3), | | |
| | | | 250 μM QUI (250 μL × 3), 250 μM DPH | | |
| | | | (250 μL × 3), 250 μM AMP (250 μL × 3), | | |
| | | | 250 μM DOP (250 μL × 3), 250 μM 4- | | |
| | | | HMA (250 μL × 3), 250 μM TYM (250 μL × | | |
| | | | 3), 250 μM 4-HA (250 μL × 3), 250 μM | | |
| | | | SER (250 μL × 3), 250 μM DOPAC (250 | | |
| | | | μL × 3), 250 μM HVA (250 μL × 3), 250 | | |
| | | | μM MPD (250 μL × 3), 100 μM ALP (250 | | |
| | | | μL × 3), 250 μM PSU (250 μL × 3), 250 | | |
| | | | μM NE (250 μL × 3), 250 μM EPI (250 μL × | | |
| | | | 3), 250 μM BPP (250 μL × 3), 250 μM | | |
| | | | METD (250 μL × 3), 250 μM MOR (250 | | |
| | | | μL × 3), 250 μM MDPV (250 μL × 3), 250 | | |
| | | | μM MDMA (250 μL × 3), 250 μM TYR + | | |
| | | | PHE (250 μL × 3) | | |
| 7 | 300 | 30 | 250 μM PRO (250 μL × 3), 250 μM LIDO | 30 | 100 |
| | | | (250 μL × 3), 250 μM CAF (250 μL × 3), | | |
| | | | 250 μM QUI (250 μL × 3), 250 μM DPH | | |
| | | | (250 μL × 3), 250 μM AMP (250 μL × 3), | | |
| | | | 250 μM DOP (250 μL × 3), 250 μM 4- | | |
| | | | HMA (250 μL × 3), 250 μM TYM (250 μL × | | |
| | | | 3), 250 μM 4-HA (250 μL × 3), 250 μM | | |
| | | | SER (250 μL × 3), 250 μM DOPAC (250 | | |
| | | | μL × 3), 250 μM HVA (250 μL × 3), 250 | | |
| | | | μM MPD (250 μL × 3), 100 μM ALP (250 | | |
| | | | μL × 3), 250 μM PSU (250 μL × 3), 250 | | |
| | | | μM NE (250 μL × 3), 250 μM EPI (250 μL × | | |
| | | | 3), 250 μM BPP (250 μL × 6), 250 μM | | |
| | | | METD (250 μL × 3), 250 μM MOR (250 | | |
| | | | μL × 3), 250 μM MDPV (250 μL × 3), 250 | | |
| | | | μM MDMA (250 μL × 3), 250 μM TYR + | | |
| | | | PHE (250 μL × 3) | | |
| 8 | 300 | 30 | 250 μM PRO (250 μL × 3), 250 μM LIDO | 30 | 100 |
| | | | (250 μL × 3), 250 μM CAF (250 μL × 3), | | |
| | | | 250 μM QUI (250 μL × 3), 250 μM DPH | | |
| | | | (250 μL × 3), 250 μM AMP (250 μL × 9), | | |
| | | | 250 μM DOP (250 μL × 3), 250 μM 4- | | |
| | | | HMA (250 μL × 12), 250 μM TYM (250 | | |
| | | | μL × 9), 250 μM 4-HA (250 μL × 9), 250 | | |
| | | | μM SER (250 μL × 3), 250 μM DOPAC | | |
| | | | (250 μL × 3), 250 μM HVA (250 μL × 3), | | |
| | | | 250 μM MPD (250 μL × 3), 100 μM ALP | | |
| | | | (250 μL × 3), 250 μM PSU (250 μL × 3), | | |
| | | | 250 μM NE (250 μL × 9), 250 μM EPI | | |
| | | | (250 μL × 6), 250 μM BPP (250 μL × 12), | | |
| | | | 250 μM METD (250 μL × 3), 250 μM | | |
| | | | MOR (250 μL × 3), 250 μM MDPV (250 | | |
| | | | μL × 3), 250 μM MDMA (250 μL × 12), | | |
| | | | 250 μM TYR + PHE (250 μL × 3) | | |
| 9 | 300 | 30 | Same as previous round | 30 | 100 |
| 10 | 300 | 30 | Same as previous round | 30 | 100 |
| 11 | 300 | 30 | Same as previous round | 30 | 100 |
| 12 | 300 | 30 | Same as previous round | 30 | 100 |

TABLE 5
Selection strategy and conditions for the fourth trial of methamphetamine SELEX.

| | Pool Size | Buffer | | Buffer | Last 3 | [Target] |
|---|---|---|---|---|---|---|
| Round | (pmol) | Wash | Counter-targets | Wash | Washes | (μM) |
| 1 | 1,000 | 9 | None | None | | 500 |
| 2 | 300 | 20 | 100 μM PRO (250 μL × 3), 100 μM LIDO | 17 | | 250 |
| | | | (250 μL × 3), 100 μM CAF (250 μL × 3), 100 | | | |
| | | | μM QUI (250 μL × 3), 100 μM DPH (250 μL × | | | |
| | | | 3), 100 μM AMP (250 μL × 6), 100 μM BPP | | | |
| | | | (250 μL × 6), 100 μM MDMA (250 μL × 6) | | | |
| 3 | 300 | 20 | 100 μM PRO (250 μL × 3), 100 μM LIDO | | | |
| | | | (250 μL × 3), 100 μM CAF (250 μL × 3), 100 | 27 | | 250 |
| | | | μM QUI (250 μL × 3), 100 μM DPH (250 μL × | | | |
| | | | 3), 100 μM AMP (250 μL × 6), 100 μM BPP | | | |
| | | | (250 μL × 6), 100 μM MDMA (250 μL × 6), | | | |
| | | | 100 μM 4-HMA(250 μL × 6), 100 μM NE | | | |
| | | | (250 μL × 6), 100 μM EPI (250 μL × 6), 100 | | | |
| | | | μM DOP(250 μL × 3), 100 μM TYM (250 μL × | | | |
| | | | 3), 100 μM 4-HA (250 μL × 3), 100 μM | | | |
| | | | TYR (250 μL × 3) | | | |
| 4 | 300 | 20 | 250 μM PRO (250 μL × 3), 250 μM LIDO | 27 | w/o | 250 |
| | | | (250 μL × 3), 250 μM CAF (250 μL × 3), 100 | | Triton | |
| | | | μM QUI (250 μL × 3), 250 μM DPH (250 μL × | | | |
| | | | 3), 100 μM AMP (250 μL × 6), 100 μM BPP | | | |
| | | | (250 μL × 6), 100 μM MDMA (250 μL × 6), | | | |
| | | | 100 μM 4-HMA(250 μL × 6), 100 μM NE | | | |
| | | | (250 μL × 6), 100 μM EPI (250 μL × 6), 100 | | | |
| | | | μM DOP(250 μL × 3), 100 μM TYM (250 μL × | | | |
| | | | 3), 100 μM 4-HA (250 μL × 3), 100 μM | | | |
| | | | TYR (250 μL × 3), 100 μM MPD (250 μL × | | | |
| | | | 3), 100 μM ALP (250 μL × 3), 100 μM PSU | | | |
| | | | (250 μL × 3), 100 μM METD (250 μL × 3), | | | |
| | | | 100 μM MOR (250 μL × 3), 100 μM MDPV | | | |
| | | | (250 μL × 3), 100 μM PHE (250 μL × 3), 100 | | | |
| | | | μM SER (250 μL × 3), 100 μM DOPAC (250 | | | |
| | | | μL × 3), 100 μM HVA (250 μL × 3) | | | |
| 5 | 300 | 24 | 250 μM PRO (250 μL × 3), 250 μM LIDO | 27 | | 100 |
| | | | (250 μL × 3), 250 μM CAF (250 μL × 3), 100 | | | |
| | | | μM QUI (250 μL × 3), 250 μM DPH (250 μL × | | | |
| | | | 3), 250 μM AMP (250 μL × 6), 250 μM BPP | | | |
| | | | (250 μL × 6), 250 μM MDMA (250 μL × 6), | | | |
| | | | 250 μM 4-HMA(250 μL × 6), 250 μM NE | | | |
| | | | (250 μL × 6), 250 μM EPI (250 μL × 6), 250 | | | |
| | | | μM DOP(250 μL × 3), 250 μM TYM (250 μL × | | | |
| | | | 3), 250 μM 4-HA (250 μL × 3), 250 μM | | | |
| | | | TYR (250 μL × 3), 250 μM MPD (250 μL × | | | |
| | | | 3), 100 μM ALP (250 μL × 3), 250 μM PSU | | | |
| | | | (250 μL × 3), 250 μM METD (250 μL × 3), | | | |
| | | | 250 μM MOR (250 μL × 3),250 μM MDPV | | | |
| | | | (250 μL × 3), 250 μM PHE (250 μL × 3), 250 | | | |
| | | | μM SER (250 μL × 3), 250 μM DOPAC (250 | | | |
| | | | μL × 3), 250 μM HVA (250 μL × 3) | | | |
| 6 | 300 | 30 | Same as previous round | 27 | | 100 |
| 7 | 300 | 30 | Same as previous round | 27 | | 100 |
| 8 | 300 | 30 | Same as previous round | 27 | | 100 |
| 9 | 300 | 30 | Same as previous round | 27 | | 100 |
| 10 | 300 | 30 | Same as previous round | 27 | | 100 |
| 11 | 300 | 30 | Same as previous round | 27 | | 100 |
| 12 | 300 | 30 | Same as previous round | 27 | | 100 |
| 13 | 300 | 30 | Same as previous round | 27 | | 100 |

PRO (Procaine),
LIDO (Lidocaine),
CAF (Caffeine),
QUI (Quinine),
DPH (Diphenhydramine),
AMP (Amphetamine),
DOPAC (3,4-Dihydroxyphenylacetic acid),
HVA (Homovanillic acid),
MPD (Methylphenidate),
ALP (Alpra]olam),
PSU ((+)-Pseudoephedrine),
NE (Norepinephrine),
EPI (Epinephrine),
BPP (Bupropion),
METD (Methadone),
MOR (Morphine),
MDPV (3,4-Methylenedioxypyrovalerone),
MDMA (3,4-Methylenedioxymethamphetamine),
DOP (Dopamine),
4-HMA (4-Hydroxy methamphetamine),
TYM (Tyramine),
4-HA (4-Hydroxy amphetamine),
SER (Serotonin),
TYR (Tyrosine),
PHE (Phenylalanine),
METH ((+)-Methamphetamine)

TABLE 6
Summary HTS statistics for methamphetamine-binding
aptamer selection from Trials 1-4.

| | | | | |
|---|---|---|---|---|
| | | | | Unique |
| | Total | Filtered | Unique | Sequences |
| Pool | Reads | Reads | Sequences | (%) |

SELEX Trial 1

| | | | | |
|---|---|---|---|---|
| Round 13 | 462,902 | 436,550 | 189,869 | 43.5 |
| Round 19 | 466,542 | 441,260 | 189,018 | 42.8 |

SELEX Trial 2

| | | | | |
|---|---|---|---|---|
| Round 9 | 533,226 | 477,697 | 185,314 | 38.8 |
| Round 11 | 449,060 | 384,057 | 82,997 | 21.6 |

SELEX Trial 3

| | | | | |
|---|---|---|---|---|
| Round 7 | 382,954 | 366,805 | 170,657 | 46.5 |
| Round 13 | 1,345,800 | 1,236,714 | 381,431 | 30.8 |
| Round 18 | 1,269,964 | 1,159,331 | 216,220 | 18.7 |

SELEX Trial 4

| | | | | |
|---|---|---|---|---|
| Round 9 | 431,950 | 392,633 | 172,750 | 44.0 |
| Round 10 | 498,324 | 447,146 | 183,069 | 40.9 |
| Round 11 | 504,238 | 460,649 | 147,713 | 32.1 |
| Round 13 | 515,898 | 476,413 | 45,953 | 9.6 |

TABLE 7
Summary of ITC experimental conditions and measurements for methamphetamine aptamer sequences.

| | | | | | | | | |
|---|---|---|---|---|---|---|---|---|
| | Titration | ΔH (kcal/ | ΔS (cal/ | | Titration | ΔH (kcal/ | ΔS (cal/ | |
| Name | time | mol) | mol · K) | K_{D} (μM) | time | mol) | mol · K) | K_{D} (μM) |

| | |
|---|---|
| (+)-Methamphetamine | Amphetamine |

SELEX Trial 1

| | | | | | | | | |
|---|---|---|---|---|---|---|---|---|
| MT1-R1 | 1× | N/A | N/A | N/A | — | — | — | — |
| MT1-R2 | 1× | N/A | N/A | N/A | — | — | — | — |
| MT1-R3 | 1× | N/A | N/A | N/A | — | — | — | — |
| MT1-R4 | 1× | N/A | N/A | N/A | — | — | — | — |
| MT1-R5 | 1× | N/A | N/A | N/A | — | — | — | — |

SELEX Trial 2

| | | | | | | | | |
|---|---|---|---|---|---|---|---|---|
| MT2-R1 | 1× | −14.9 | −24.7 | 2.5 ± 0.1 | 1× | −14.8 | −25.9 | 5.7 ± 0.2 |
| MT2-R2 | 1× | −20.9 | −46.2 | 5.0 ± 0.1 | — | — | — | — |
| MT2-R3 | 1× | −6.82 | −1.3 | 18.0 ± 1.1 | — | — | — | — |
| MT2-R4 | 1× | −6.1 | −2.3 | 10.1 ± 0.7 | — | — | — | — |

SELEX Trial 3

| | | | | | | | | |
|---|---|---|---|---|---|---|---|---|
| M1 | 1× | −10.3 | −10.7 | 5.1 ± 0.5 | 1× | −12.4 | −18.8 | 9.1 ± 0.5 |
| M2 | 1× | −9.9 | −8.5 | 3.6 ± 0.3 | 1× | −11.1 | −13.6 | 6.6 ± 0.4 |
| M3 | 1× | −12.5 | −17.9 | 4.9 ± 0.5 | 1× | −4.8 | 2.5 | 78.5 ± 4.7 |
| M4 | 1× | −9.2 | −8.9 | 12.4 ± 0.8 | 1× | −9.0 | −9.1 | 22.9 ± 1.5 |
| M5 | 1× | −6.2 | 3.2 | 5.3 ± 0.5 | 1× | −2.5 | 10.9 | 56.4 ± 3.2 |
| M6 | 1× | −12.1 | −16.4 | 4.4 ± 0.2 | 1× | −11.8 | −15.9 | 6.4 ± 0.2 |
| M7 | 1× | −8.6 | −5.7 | 6.0 ± 0.5 | 1× | −4.1 | 7.0 | 27.2 ± 1.1 |
| M8 | 1× | −6.5 | 1.3 | 8.5 ± 0.8 | 1× | −3.7 | 9.0 | 19.3 ± 1.5 |
| M9 | 1× | −8.3 | −4.1 | 6.1 ± 0.4 | 1× | −7.1 | −5.6 | 89.1 ± 6.2 |
| M11 | 1× | −20.7 | −46.7 | 8.9 ± 0.1 | 2× | −6.9 | −5.7 | 127 ± 6.8 |
| M12 | 1× | −14.3 | −24.8 | 7.5 ± 0.2 | 2× | −19.2 | −47.3 | 171 ± 16.1 |
| M13 | 1× | −10.1 | −8.9 | 3.3 ± 0.1 | 1× | −4.6 | 5.9 | 20.0 ± 0.9 |
| M14 | 1× | −14.5 | −24.1 | 3.7 ± 0.2 | 1× | −12.9 | −20.0 | 7.6 ± 0.5 |
| M18 | 1× | −7.0 | −0.1 | 7.0 ± 0.6 | 1× | −3.1 | 8.9 | 60.3 ± 3.9 |
| M20 | 1× | −6.8 | 0.7 | 6.4 ± 0.3 | 2× | −2.9 | 9.6 | 60.6 ± 2.9 |
| M21 | 1× | −6.7 | 0.8 | 6.9 ± 0.3 | 2× | −3.5 | 7.1 | 70.8 ± 3.1 |

SELEX Trial 4

| | | | | | | | | |
|---|---|---|---|---|---|---|---|---|
| ML1 | 1× | −42.8 | −119.2 | 3.29 ± 0.11 | 1× | −27.4 | −73.6 | 67.2 ± 1.09 |
| ML2 | 1× | −31.4 | −81.4 | 4.44 ± 0.15 | 1× | −15.2 | −31.5 | 45.3 ± 1.25 |
| ML3 | 1× | −12.7 | −16.2 | 1.54 ± 0.09 | 2× | −4.84 | 2.4 | 80.6 ± 3.13 |
| ML4 | 1× | −19.2 | −39.2 | 2.53 ± 0.08 | 2× | −9.1 | −12.7 | 117 ± 4.1 |
| ML5 | 1× | −13.1 | −17.8 | 1.55 ± 0.07 | 1× | −10.9 | −12.7 | 5.25 ± 0.26 |
| ML6 | 1× | −41.6 | −114.5 | 2.09 ± 0.08 | 1× | −25 | −64.1 | 40.5 ± 0.59 |
| ML7 | 1× | −24.6 | −56.0 | 1.29 ± 0.03 | 1× | −12.7 | −21.1 | 18.0 ± 0.55 |
| ML8 | 1× | −9.8 | −9.8 | 7.98 ± 0.39 | 1× | −6.71 | −2.7 | 43.7 ± 1.83 |
| ML10 | 1× | −40 | −109.0 | 1.88 ± 0.07 | 1× | −25 | −64.5 | 43.0 ± 1.57 |
| ML11 | 1× | −43 | −119.5 | 2.45 ± 0.08 | 1× | −25.6 | −66.5 | 46.6 ± 0.65 |
| ML12 | 1× | −7.2 | −0.3 | 5.67 ± 0.28 | 1× | −8.41 | −6.7 | 18.2 ± 0.53 |
| ML13 | 1× | −34.5 | −92.9 | 7.01 ± 0.36 | 2× | −24.5 | −65.2 | 140 ± 3.9 |

TABLE 8
ITC results and conditions for determining affinity
of methamphetamine aptamer sequences.

| | [Aptamer] | [(+)-METH] | ΔH (kcal/ | ΔS (cal/ | | |
|---|---|---|---|---|---|---|
| Aptamer | (μM) | (μM) | mol) | mol · K) | K_{D} (μM) | Buffer |
| ML3 | 50 | 1500 | −20.0 | −47 | 34.3 ± 1.9 | 0.5× PBS, 1 mM |
| | | | | | | MgCl₂, 0.005% |
| | | | | | | Triton X-100 |
| | 50 | 1250 | −13.5 | −23 | 9.9 ± 0.6 | 0.5× PBS, 2 mM |
| | | | | | | MgCl₂, 0.005% |
| | | | | | | Triton X-100 |
| | 20 | 500 | −12.7 | −16 | 1.5 ± 0.1 | 0.5× PBS, 5 mM |
| | | | | | | MgCl₂, 0.005% |
| | | | | | | Triton X-100 |
| ML4 | 50 | 1500 | −47.4 | −141 | 65.2 ± 1.8 | 0.5× PBS, 1 mM |
| | | | | | | MgCl₂, 0.005% |
| | | | | | | Triton X-100 |
| | 50 | 1250 | −22.2 | −53 | 16.2 ± 0.5 | 0.5× PBS, 2 mM |
| | | | | | | MgCl₂, 0.005% |
| | | | | | | Triton X-100 |
| | 20 | 500 | −19.2 | −39 | 2.5 ± 0.1 | 0.5× PBS, 5 mM |
| | | | | | | MgCl₂, 0.005% |
| | | | | | | Triton X-100 |
| ML3-mut1 | 50 | 2000 | −4.4 | 0.5 | 408 ± 16 | 0.5× PBS, 5 mM |
| ML3-mut2 | 50 | 2000 | −5.4 | 2.1 | 39.0 ± 1.8 | MgCl₂, 0.005% |
| ML3-mut3 | 50 | 2000 | −10.9 | −10 | 1.4 ± 0.1 | Triton X-100 |
| ML3-mut4 | 50 | 2000 | −5.1 | 1.6 | 72.5 ± 3.1 | |
| ML3-mut5 | 50 | 2000 | NA | NA | NA | |
| ML3-mut6 | 50 | 2000 | NA | NA | NA | |
| ML4-mut1 | 50 | 2000 | −9.9 | −14 | 67.1 ± 4.1 | 0.5× PBS, 5 mM |
| ML4-mut2 | 50 | 2000 | −12.1 | −16 | 4.0 ± 0.1 | MgCl₂, 0.005% |
| ML4-mut3 | 50 | 2000 | −15.2 | −26 | 3.2 ± 0.1 | Triton X-100 |
| ML4-mut4 | 50 | 2000 | −16.2 | −30 | 4.9 ± 0.2 | |
| ML4-mut5 | 50 | 2000 | −16.5 | −31 | 3.3 ± 0.1 | |
| ML4-mut6 | 50 | 2000 | −14.6 | −24 | 3.8 ± 0.1 | |
| ML4-mut7 | 50 | 2000 | −8.4 | −9 | 64.3 ± 3.9 | |

TABLE 9
List of methamphetamine aptamer sequences.

| Name | Sequence |
|---|---|
| 84-nt | ACTGGAGCTCAATCAGTACACGACGGTTGCAAGTGGGACTCTGGTA |
| aptaMETH | GGCTGGGTTAATTTGGGACAAGCTTCAACCATGGAGTA (SEQ ID |
| | NO: 14) |
| 38-nt | ACGGTTGCAAGTGGGACTCTGGTAGGCTGGGTTAATTTGG (SEQ ID |
| aptaMETH | NO: 15) |
| Aptamer-2 | AGGAATTCAGATCTCCCTGCAGGTGGTGTTTTTTTTGTGTGCTGTGTT |
| | TTTGTGATGCATGCTCGAGGAGCTCAGGATCCCG (SEQ ID NO: 16) |
| Apta-4 | ATACGAGCTTGTTCAATAGCGTTTCTATCTGGCTGTATCGTGATAGT |
| | AAGAGCACTAATGATAGTAAGAGCAATC (SEQ ID NO: 17) |
| MT1-R1 | GGGACGACCCCATTAGTGGTGCCGGGTGTTTATGGTGCGTCGTCCC |
| | (SEQ ID NO: 18) |
| MT1-R2 | GGGACGACCACTTTGGGCAGGTAGGTTGGGCAAGTAGTGTCGTCCC |
| | (SEQ ID NO: 19) |
| MT1-R3 | GGGACGACCCAGTTGGGTAGGTATGGTTGGGCACGGTGGTCGTCCC |
| | (SEQ ID NO: 20) |
| MT1-R4 | GGGACGACGGCACGGTTAGCATGGCTGTATTTGTGTAGGTCGTCCC |
| | (SEQ ID NO: 21) |
| MT1-R5 | GGGACGACCACGGTTAAAGTAGCACTTTGTATTTGGGTGTCGTCCC |
| | (SEQ ID NO: 22) |
| MT2-R1 | GGGACGACGCTAGGGGATCTTTTGGGGTTTGTATGGGCGTCGTCCC |
| | (SEQ ID NO: 23) |
| MT2-R2 | GGGACGACGAGGGGATCCATCCAATGGGGTTTGTATACGTCGTCCC |
| | (SEQ ID NO: 24) |
| MT2-R3 | GGGACGACGGTACCCGCCGGTGCAGGATCTGGGGTGGTGTCGTCCC |
| | (SEQ ID NO: 25) |
| MT2-R4 | GGGACGACGGCAATTGCCGGTGCAGGATCTGAATTATCGTCGTCCC |
| | (SEQ ID NO: 26) |
| M1 | GGGACGACGGGATGGTAAGTGGTAGGTCGTTGGAATTTCTACTGTA |
| | TCGTCGTCCC (SEQ ID NO: 27) |
| M2 | GGGACGACGGGACGGTAAGTGGTAGGTCGTTGGAATTTCTACTGTA |
| | TCGTCGTCCC (SEQ ID NO: 28) |
| M3 | GGGACGACCGTCCGAGTTTTAGGAGTGGTTGCATTGCGCTGGGAAG |
| | AGGTCGTCCC (SEQ ID NO: 29) |
| M4 | GGGACGACGCACTATGGCTAGGGTTGGTACGGTCGTGAACCGCGGG |
| | GTGTCGTCCC (SEQ ID NO: 30) |
| M5 | GGGACGACGGCACGGGGTTATTCGTTGGTTGCACTGCGCTGGGAGG |
| | CAGTCGTCCC (SEQ ID NO: 31) |
| M6 | GGGACGACGGGATGGTAAGTGGCAGGTCGTTGGAATTTCTACTGTA |
| | TCGTCGTCCC (SEQ ID NO: 32) |
| M7 | GGGACGACGGCACGAGGTTATTCGTTGGTTGCACTGCGCTGGGAGG |
| | TAGTCGTCCC (SEQ ID NO: 33) |
| M8 | GGGACGACGGGGCTCTTACCCTGGAGGGTAGAAGGGGAGGTGTGGT |
| | CAGTCGTCCC (SEQ ID NO: 34) |
| M9 | GGGACGACGGCACGTGGTTATTCGTTGGTTGCACTGCGCTGGGAGG |
| | CAGTCGTCCC (SEQ ID NO: 35) |
| M11 | GGGACGACCGGCTGTCTGGATGCATTGCGCCGGGAACTCGGACGGA |
| | TGGTCGTCCC (SEQ ID NO: 36) |
| M12 | GGGACGACCGTCCGAGTTATAGGAGTGGTTGCATTGCGCTGGGAAG |
| | AGGTCGTCCC (SEQ ID NO: 37) |
| M13 | GGGACGACGGCACGAGGTTATTCGTTGGTTGCACTGCGCTGGGAGG |
| | AAGTCGTCCC (SEQ ID NO: 38) |
| M14 | GGGACGACGGGACGGTAAGTGGCAGGTCGTTGGAATTTCTACTGTA |
| | TCGTCGTCCC (SEQ ID NO: 39) |
| M18 | GGGACGACGGCACGAGGTTATTCGTTGGTTGCACTGCGCTGGGAGG |
| | CAGTCGTCCC (SEQ ID NO: 40) |
| M20 | GGGACGACGGCACGAGGTTAATCGTTGGTTGCACTGCGCTGGGAGG |
| | CAGTCGTCCC (SEQ ID NO: 41) |
| M21 | GGGACGACGGCACGAGGTAATTCGTTGGTTGCACTGCGCTGGGAGG |
| | CAGTCGTCCC (SEQ ID NO: 42) |
| ML1 | TTCAAATCTGAGCAGCCTAGTTACTGACGGGTTTATAGTACTCGCAT |
| | TGAGTCTAGATTTGAA (SEQ ID NO: 43) |
| ML2 | TTCAAATCTGGTGCCCTTTCGCAGTCGGTTGGTAATGAGACCACTTT |
| | AGAGTCTAGATTTGAA (SEQ ID NO: 44) |
| ML3 | TTCAAATCTTATGTACGTTCTATCGCATGGATGGGATTTTGGGTTAC |
| | CTAGTCTAGATTTGAA (SEQ ID NO: 45) |
| ML4 | TTCAAATCTTGGTACGTTCTGCCTTTTCTTATTTTGCTTTGGGTTACCT |
| | AGTCTAGATTTGAA (SEQ ID NO: 46) |
| ML5 | TTCAAATCTATGGTGCAGTGCGCAGGGACGGAGGTAAAACATGAGT |
| | TTTAGTCTAGATTTGAA |
| | (SEQ ID NO: 47) |
| ML6 | TTCAAATCTGAGCAGCCTAGTTACTGACGGGTTTATAGTACTCGCAT |
| | CGAGTCTAGATTTGAA (SEQ ID NO: 48) |
| ML7 | TTCAAATCTGGTGCCCTTTCGCAGTCGGTTGGTAATGAGATCACTTT |
| | AGAGTCTAGATTTGAA (SEQ ID NO: 49) |
| ML8 | TTCAAATCTCCACGCTGGACTGGATGCAATGCGCCGGGATATCGTCT |
| | AAAGTCTAGATTTGAA (SEQ ID NO: 50) |
| ML10 | TTCAAATCTGAGCAGCCTAGTTACTGACGGGTTTATAGTACTCACAT |
| | TGAGTCTAGATTTGAA (SEQ ID NO: 51) |
| ML11 | TTCAAATCTGAGCAGCCTAGTTACTGACGGGTTTATAGTACTCGTAT |
| | TGAGTCTAGATTTGAA (SEQ ID NO: 52) |
| ML12 | TTCAAATCTGCGGCGACTAATTCTTAGTGGTGGTTGCAATGCGCTGG |
| | GATGAGTCTAGATTTGAA (SEQ ID NO: 53) |
| ML13 | TTCAAATCTGAGCAGCCTAGTTACTGACGGGTTTATAGTACTCGCAC |
| | TGAGTCTAGATTTGAA (SEQ ID NO: 54) |

TABLE 10
List of point-mutant sequences of methamphetamine aptamer.

| NAME | Sequence (5′-3′) |
|---|---|
| ML3-mut1 | TTCAAATCTTATGTAAGTTCTATCGCATGGATGGGATTTTGGGTTAC |
| | CTAGTCTAGATTTGAA (SEQ ID NO: 55) |
| ML3-mut2 | TTCAAATCTTATGTACGTACTATCGCATGGATGGGATTTTGGGTTAC |
| | CTAGTCTAGATTTGAA (SEQ ID NO: 56) |
| ML3-mut3 | TTCAAATCTTATGTACGTTCTATCGCATGGATGTGATTTTGGGTTACC |
| | TAGTCTAGATTTGAA (SEQ ID NO: 57) |
| ML3-mut4 | TTCAAATCTTATGTACGTTCTATCGCATGGATGGGATTATGGGTTAC |
| | CTAGTCTAGATTTGAA (SEQ ID NO: 58) |
| ML3-mut5 | TTCAAATCTTATGTACGTTCTATCGCATGGATGGGATTTTTGGTTACC |
| | TAGTCTAGATTTGAA (SEQ ID NO: 59) |
| ML3-mut6 | TTCAAATCTTATGTACGTTCTATCGCATGGATGGGATTTTGGGTTACC |
| | AAGTCTAGATTTGAA (SEQ ID NO: 60) |
| ML4-mut1 | TTCAAATCTTGGTACGTACTGCCTTTTCTTATTTTGCTTTGGGTTACC |
| | TAGTCTAGATTTGAA (SEQ ID NO: 61) |
| ML4-mut2 | TTCAAATCTTGGTACGTTCTGCATTTTCTTATTTTGCTTTGGGTTACCT |
| | AGTCTAGATTTGAA (SEQ ID NO: 62) |
| ML4-mut3 | TTCAAATCTTGGTACGTTCTGCCTATTCTTATTTTGCTTTGGGTTACC |
| | TAGTCTAGATTTGAA (SEQ ID NO: 63) |
| ML4-mut4 | TTCAAATCTTGGTACGTTCTGCCTTTACTTATTTTGCTTTGGGTTACC |
| | TAGTCTAGATTTGAA (SEQ ID NO: 64) |
| ML4-mut5 | TTCAAATCTTGGTACGTTCTGCCTTTTCTTATATTGCTTTGGGTTACC |
| | TAGTCTAGATTTGAA (SEQ ID NO: 65) |
| ML4-mut6 | TTCAAATCTTGGTACGTTCTGCCTTTTCTTATTTAGCTTTGGGTTACC |
| | TAGTCTAGATTTGAA (SEQ ID NO: 66) |
| ML4-mut7 | TTCAAATCTTGGTACGTTCTGCCTTTTCTTATTTTGCTATGGGTTACC |
| | TAGTCTAGATTTGAA (SEQ ID NO: 67) |

SEQUENCES

SEQ ID NO: 1 (Trial 1 &2 Library)
GGAGGCTCTCGGGACGAC(N30)GTCGTCCCGCCTTTAGGATTTACAG
SEQ ID NO: 2 (Trial 3 Library)
GGAGGCTCTCGGGACGAC(N40)GTCGTCCCGCCTTTAGGATTTACAG
SEQ ID NO: 3 (Trial 4 library)
TCCATAGCTCCTTGTTCAAATCT(N40)AGTCTAGATTTGAATTCTCGGATACAACGA
SEQ ID NO: 4 (Trial 1-3 FP)
GGAGGCTCTCGGGACGAC
SEQ ID NO: 5 (Trial 1-3 RP-bio)
/5Bio/-CTGTAAATCCTAAAGGCGGGACGAC
SEQ ID No: 6 (Trial 1-3 cDNA 15-bio)
GTCGTCCCGAGAGCCATA-/3Bio/
SEQ ID No: 7 (Trial 1-3 FP-HTS)
ACACTCTTTCCCTACACGACGCTCTTCCGATCT(N45)GGAGGCTCTCGGGACGAC
SEQ ID NO: 8 (Trial RP-HTS)
GACTGGAGTTCAGACGTGTGCTCTTCCGATCT(N43)CTGTAAATCCTAAAGGCGGGA
CGAC
SEQ ID No: 9 (Trial 4 FP)
TCCATAGCTCCTTGTTCAAATCT
SEQ ID NO: 10 (Trial 4 RP-bio)
/5Bio/TCGTTGTATCCGAGAATTCAAATCT
SEQ ID No: 11 (Trial 4 cDNA15-bio)
/5Bio/CGAGAATTCAAATCT
SEQ ID No: 12 (Trial 4 FP-HTS)
ACACTCTTTCCCTACACGACGCTCTTCCGATCT(N44)TCCATAGCTCCTTGTTCAAA
SEQ ID No: 13 (Trial 4 RP-HTS)
GACTGGAGTTCAGACGTGTGCTCTTCCGATCT(N45)TCGTTGTATCCGAGAATTCA
SEQ ID NO: 14 (84-nt aptaMETH)
ACTGGAGCTCAATCAGTACACGACGGTTGCAAGTGGGACTCTGGTAGGCTGGGTTA
ATTTGGGACAAGCTTCAACCATGGAGTA
SEQ ID NO: 15 (38-nt aptaMETH)
ACGGTTGCAAGTGGGACTCTGGTAGGCTGGGTTAATTTGG
SEQ ID NO: 16 (Aptamer-2)
AGGAATTCAGATCTCCCTGCAGGTGGTGTTTTTTTTGTGTGCTGTGTTTTTGTGATGC
ATGCTCGAGGAGCTCAGGATCCCG
SEQ ID NO: 17 (Apta-4)
ATACGAGCTTGTTCAATAGCGTTTCTATCTGGCTGTATCGTGATAGTAAGAGCACTA
ATGATAGTAAGAGCAATC
SEQ ID NO: 18 (MT1-R1)
GGGACGACCCCATTAGTGGTGCCGGGTGTTTATGGTGCGTCGTCCC
SEQ ID NO: 19 (MT1-R2)
GGGACGACCACTTTGGGCAGGTAGGTTGGGCAAGTAGTGTCGTCCC
SEQ ID NO: 20 (MT1-R3)
GGGACGACCCAGTTGGGTAGGTATGGTTGGGCACGGTGGTCGTCCC
SEQ ID NO: 21 (MT1-R4)
GGGACGACGGCACGGTTAGCATGGCTGTATTTGTGTAGGTCGTCCC
SEQ ID NO: 22 (MT1-R5)
GGGACGACCACGGTTAAAGTAGCACTTTGTATTTGGGTGTCGTCCC
SEQ ID NO: 23 (MT2-R1)
GGGACGACGCTAGGGGATCTTTTGGGGTTTGTATGGGCGTCGTCCC
SEQ ID NO: 24 (MT2-R2)
GGGACGACGAGGGGATCCATCCAATGGGGTTTGTATACGTCGTCCC
SEQ ID NO: 25 (MT2-R3)
GGGACGACGGTACCCGCCGGTGCAGGATCTGGGGTGGTGTCGTCCC
SEQ ID NO: 26 (MT2-R4)
GGGACGACGGCAATTGCCGGTGCAGGATCTGAATTATCGTCGTCCC
SEQ ID NO: 27 (M1)
GGGACGACGGGATGGTAAGTGGTAGGTCGTTGGAATTTCTACTGTATCGTCGTCCC
SEQ ID NO: 28 (M2)
GGGACGACGGGACGGTAAGTGGTAGGTCGTTGGAATTTCTACTGTATCGTCGTCCC
SEQ ID NO: 29 (M3)
GGGACGACCGTCCGAGTTTTAGGAGTGGTTGCATTGCGCTGGGAAGAGGTCGTCCC
SEQ ID NO: 30 (M4)
GGGACGACGCACTATGGCTAGGGTTGGTACGGTCGTGAACCGCGGGGTGTCGTCCC
SEQ ID NO: 31 (M5)
GGGACGACGGCACGGGGTTATTCGTTGGTTGCACTGCGCTGGGAGGCAGTCGTCCC
SEQ ID NO: 32 (M6)
GGGACGACGGGATGGTAAGTGGCAGGTCGTTGGAATTTCTACTGTATCGTCGTCCC
SEQ ID NO: 33 (M7)
GGGACGACGGCACGAGGTTATTCGTTGGTTGCACTGCGCTGGGAGGTAGTCGTCCC
SEQ ID NO: 34 (M8)
GGGACGACGGGGCTCTTACCCTGGAGGGTAGAAGGGGAGGTGTGGTCAGTCGTCCC
SEQ ID NO: 35 (M9)
GGGACGACGGCACGTGGTTATTCGTTGGTTGCACTGCGCTGGGAGGCAGTCGTCCC
SEQ ID NO: 36 (M11)
GGGACGACCGGCTGTCTGGATGCATTGCGCCGGGAACTCGGACGGATGGTCGTCCC
SEQ ID NO: 37 (M12)
GGGACGACCGTCCGAGTTATAGGAGTGGTTGCATTGCGCTGGGAAGAGGTCGTCCC
SEQ ID NO: 38 (M13)
GGGACGACGGCACGAGGTTATTCGTTGGTTGCACTGCGCTGGGAGGAAGTCGTCCC
SEQ ID NO: 39 (M14)
GGGACGACGGGACGGTAAGTGGCAGGTCGTTGGAATTTCTACTGTATCGTCGTCCC
SEQ ID NO: 40 (M18)
GGGACGACGGCACGAGGTTATTCGTTGGTTGCACTGCGCTGGGAGGCAGTCGTCCC
SEQ ID NO: 41 (M20)
GGGACGACGGCACGAGGTTAATCGTTGGTTGCACTGCGCTGGGAGGCAGTCGTCCC
SEQ ID NO: 42 (M21)
GGGACGACGGCACGAGGTAATTCGTTGGTTGCACTGCGCTGGGAGGCAGTCGTCCC
SEQ ID NO: 43 (ML1)
TTCAAATCTGAGCAGCCTAGTTACTGACGGGTTTATAGTACTCGCATTGAGTCTAGA
TTTGAA
SEQ ID NO: 44 (ML2)
TTCAAATCTGGTGCCCTTTCGCAGTCGGTTGGTAATGAGACCACTTTAGAGTCTAGA
TTTGAA
SEQ ID NO: 45 (ML3)
TTCAAATCTTATGTACGTTCTATCGCATGGATGGGATTTTGGGTTACCTAGTCTAGAT
TTGAA
SEQ ID NO: 46 (ML4)
TTCAAATCTTGGTACGTTCTGCCTTTTCTTATTTTGCTTTGGGTTACCTAGTCTAGATT
TGAA
SEQ ID NO: 47 (ML5)
TTCAAATCTATGGTGCAGTGCGCAGGGACGGAGGTAAAACATGAGTTTTAGTCTAG
ATTTGAA
SEQ ID NO: 48 (ML6)
TTCAAATCTGAGCAGCCTAGTTACTGACGGGTTTATAGTACTCGCATCGAGTCTAGA
TTTGAA
SEQ ID NO: 49 (ML7)
TTCAAATCTGGTGCCCTTTCGCAGTCGGTTGGTAATGAGATCACTTTAGAGTCTAGA
TTTGAA
SEQ ID NO: 50 (ML8)
TTCAAATCTCCACGCTGGACTGGATGCAATGCGCCGGGATATCGTCTAAAGTCTAGA
TTTGAA
SEQ ID NO: 51 (ML10)
TTCAAATCTGAGCAGCCTAGTTACTGACGGGTTTATAGTACTCACATTGAGTCTAGA
TTTGAA
SEQ ID NO: 52 (ML11)
TTCAAATCTGAGCAGCCTAGTTACTGACGGGTTTATAGTACTCGTATTGAGTCTAGA
TTTGAA
SEQ ID NO: 53 (ML12)
TTCAAATCTGCGGCGACTAATTCTTAGTGGTGGTTGCAATGCGCTGGGATGAGTCTA
GATTTGAA
SEQ ID NO: 54 (ML13)
TTCAAATCTGAGCAGCCTAGTTACTGACGGGTTTATAGTACTCGCACTGAGTCTAGA
TTTGAA
SEQ ID NO: 55 (ML3-mut1)
TTCAAATCTTATGTAAGTTCTATCGCATGGATGGGATTTTGGGTTACCTAGTCTAGAT
TTGAA
SEQ ID NO: 56 (ML3-mut2)
TTCAAATCTTATGTACGTACTATCGCATGGATGGGATTTTGGGTTACCTAGTCTAGAT
TTGAA
SEQ ID NO: 57 (ML3-mut3)
TTCAAATCTTATGTACGTTCTATCGCATGGATGTGATTTTGGGTTACCTAGTCTAGAT
TTGAA
SEQ ID NO: 58 (ML3-mut4)
TTCAAATCTTATGTACGTTCTATCGCATGGATGGGATTATGGGTTACCTAGTCTAGAT
TTGAA
SEQ ID NO: 59 (ML3-mut5)
TTCAAATCTTATGTACGTTCTATCGCATGGATGGGATTTTTGGTTACCTAGTCTAGAT
TTGAA
SEQ ID NO: 60 (ML3-mut6)
TTCAAATCTTATGTACGTTCTATCGCATGGATGGGATTTTGGGTTACCAAGTCTAGAT
TTGAA
SEQ ID NO: 61 (ML4-mut1)
TTCAAATCTTGGTACGTACTGCCTTTTCTTATTTTGCTTTGGGTTACCTAGTCTAGATT
TGAA
SEQ ID NO: 62 (ML4-mut2)
TTCAAATCTTGGTACGTTCTGCATTTTCTTATTTTGCTTTGGGTTACCTAGTCTAGATT
TGAA
SEQ ID NO: 63 (ML4-mut3)
TTCAAATCTTGGTACGTTCTGCCTATTCTTATTTTGCTTTGGGTTACCTAGTCTAGATT
TGAA
SEQ ID NO: 64 (ML4-mut4)
TTCAAATCTTGGTACGTTCTGCCTTTACTTATTTTGCTTTGGGTTACCTAGTCTAGATT
TGAA
SEQ ID NO: 65 (ML4-mut5)
TTCAAATCTTGGTACGTTCTGCCTTTTCTTATATTGCTTTGGGTTACCTAGTCTAGATT
TGAA
SEQ ID NO: 66 (ML4-mut6)
TTCAAATCTTGGTACGTTCTGCCTTTTCTTATTTAGCTTTGGGTTACCTAGTCTAGATT
TGAA
SEQ ID NO: 67 (ML4-mut7)
TTCAAATCTTGGTACGTTCTGCCTTTTCTTATTTTGCTATGGGTTACCTAGTCTAGATT
TGAA
SEQ ID NO: 68 (Aptamer for EAB sensor: L13-45-MB)
5ThioMC6-D/TATGTACGTTCTATCGCATGGATGGGATTTTGGGTTACCTAGTCT/3MeBlN

What is claimed is:

1. An aptamer comprising:
(a) a nucleic acid that specifically binds to a phenethylamine-class compound, wherein the aptamer exhibits less than 25% cross-reactivity to a structural analog or an enantiomer of the phenethylamine-class compound; and
(b) a detectable signal, wherein the signal is detectable upon binding of the nucleic acid to the phenethylamine-class compound.

2. The aptamer of claim 1, wherein the phenethylamine-class compound is (+)-methamphetamine.

3. The aptamer of claim 1, wherein the aptamer comprises a nucleic acid sequence as set forth in any one of SEQ ID NOS: 45, 46, 57, 64, 65, and 66.

4. The aptamer of claim 3, wherein the nucleic acid sequence comprises at least one additional mutation and/or at least one additional non-natural nucleic acid.

5. The aptamer of claim 4, wherein the at least one additional mutation of a thymine at a position corresponding to T18 or T39 within the nucleic acid sequence decreases affinity for the phenethylamine-class compound binding by at least 20-fold.

6. The aptamer of claim 1, wherein the nucleic acid comprises a randomized region that includes at least 40 nucleotides, wherein the at least 40 nucleotides are thymine rich with a thymine content of at least 40 percent.

7. The aptamer of claim 1, wherein the detectable signal is an optically visible signal or an electrochemical signal.

8. The aptamer of claim 1, wherein the optically visible signal comprises a colorimetric dye which is detectable upon displacement.

9. A method for detecting presence of a phenethylamine-class compound in a sample, the method comprising:
(a) providing the aptamer of claim 1 in the presence of the phenethylamine-class compound; and
(b) measuring output signal, thereby detecting the presence of the phenethylamine-class compound.

10. The method of claim 9, wherein the sample comprises a seized material or a biological fluid, wherein the biological fluid comprises saliva, blood, serum, plasma, or urine.

11. The method of claim 9, wherein the analyte is (+)-methamphetamine.

12. The aptamer of claim 9, wherein the detectable signal is an optically visible signal or an electrochemical signal.

13. The aptamer of claim 9, wherein the optically visible signal comprises a colorimetric dye which is detectable upon displacement.

14. A screening assay for determining presence of a phenethylamine-class compound in a sample, the screening assay comprising the aptamer of claim 1.

15. The aptamer of claim 1, wherein the detectable signal comprises a colorimetric dye, wherein the colorimetric dye comprises X-732-91B.

16. The method of claim 9, wherein the method provides the aptamer and a detectable signal, wherein the detectable signal comprises a colorimetric dye, wherein the colorimetric dye comprises X-732-91B.

17. An aptamer comprising:
a) a nucleic acid aptamer that specifically binds to a target compound; and
b) a detectable signal, wherein the detectable signal comprises a colorimetric dye comprising X-732-91B.

18. A method for detecting presence of a target compound in a sample, the method comprising:
a) providing the composition of claim 17 in the presence of the target compound; and
b) measuring output signal, thereby detecting the presence of the target compound.

19. The method of claim 18, wherein the sample comprises a seized material or a biological fluid, wherein the biological fluid comprises saliva, blood, serum, plasma, or urine.

20. A screening assay for determining presence of a target compound in a sample, the screening assay comprising the composition of claim 17.
